(19) United States
(12) Patent Application Publication
(10) Pub. No.: US 2026/0138979 A1
(43) Pub. Date: May 21, 2026

(54) PROCESSES FOR PREPARING KRAS INHIBITORS

(71) Applicant: Incyte Corporation, Wilmington, DE (US)

(72) Inventors: Wenxing GUO, Wilmington, DE (US); Jiemin HU, Wilmington, DE (US); Zhongjiang JIA, Wilmington, DE (US); Michelle KROC, Wilmington, DE (US); Minyan LI, Wilmington, DE (US); Yi LI, Wilmington, DE (US); Qiyan LIN, Wilmington, DE (US); Pingli LIU, Wilmington, DE (US); Timothy MARTIN, Wilmington, DE (US); Trupti SHETH, Wilmington, DE (US); Bardia SOLTANZADEH, Wilmington, DE (US); Naijing SU, Wilmington, DE (US); Joseph TASSONE, Wilmington, DE (US); Michael XIA, Wilmington, DE (US); Jinya YIN, Wilmington, DE (US); Jiacheng ZHOU, Wilmington, DE (US); Xiaotao Shaw PU, Wilmington, DE (US); Wayne HAN, Wilmington, DE (US)

(21) Appl. No.: 19/366,223

(22) Filed: Oct. 22, 2025

Related U.S. Application Data
(60) Provisional application No. 63/711,016, filed on Oct. 23, 2024.

(51) Int. Cl. C07D 471/04; A61K 9/20; A61K 31/4745

(52) U.S. Cl. CPC C07D 471/04; A61K 9/2009; A61K 9/2013; A61K 9/2018; A61K 9/2054; A61K 31/4745

(57) ABSTRACT

This disclosure provides crystalline, salt forms of a KRAS inhibitor, pharmaceutical compositions comprising a KRAS inhibitor, and efficient and scalable processes for preparing a KRAS inhibitor.

### RELATED APPLICATIONS

This application is related to U.S. Provisional Application No. 63/711,016, filed Oct. 23, 2024, the content of which is incorporated in its entirety.

### BACKGROUND

Ras proteins are part of the family of small GTPases that are activated by growth factors and various extracellular stimuli. The Ras family regulates intracellular signaling pathways responsible for growth, migration, survival, and differentiation of cells. Activation of Ras proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (D. Simanshu, et al., Cell, 2017, 170(1), 17-33).

The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al. Nat. Rev. Drug. Discov., 2014, 13(11), 828-51). Most RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) while KRAS G12V mutations are associated with pancreatic cancers (30%), followed by colorectal adenocarcinomas (27%), and lung adenocarcinomas (23%) (A. D. Cox, et al. Nat. Rev. Drug. Discov., 2014, 13(11), 828-51). In contrast, KRAS G12C mutations predominate in non-small cell lung cancer (NSCLC) comprising 11-16% of lung adenocarcinomas, and 2-5% of pancreatic and colorectal adenocarcinomas (A. D. Cox, et al. Nat. Rev. Drug. Discov., 2014, 13(11), 828-51). Genomic studies across hundreds of cancer cell lines have demonstrated that cancer cells harboring KRAS mutations are highly dependent on KRAS function for cell growth and survival (R. McDonald, et al., Cell, 2017, 170(3), 577-92). The role of mutant KRAS as an oncogenic driver is further supported by extensive in vivo experimental evidence showing mutant KRAS is required for early tumor onset and maintenance in animal models (A. D. Cox, et al. Nat. Rev. Drug. Discov., 2014, 13(11), 828-51).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

Efficient and scalable synthetic routes are required to prepare KRAS inhibitors. The processes disclosed herein meet this need by providing a scalable synthetic route to prepare chiral KRAS inhibitors.

### SUMMARY

Provided herein are processes for preparing KRAS inhibitors such as compounds of

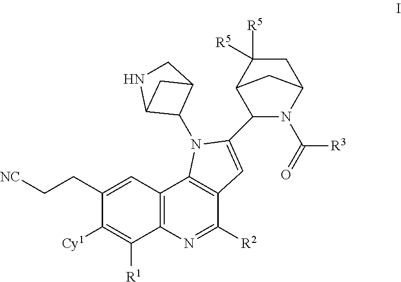

or pharmaceutically acceptable salts thereof, wherein the variables are as disclosed herein. Also disclosed are intermediates useful for preparing such KRAS inhibitors and processes of preparing such intermediates. In addition, various pharmaceutically acceptable salts and crystal forms of Formula I are disclosed herein.

### BRIEF DESCRIPTION OF THE DRAWINGS

FIG. 1 shows the XRPD diffractogram of Compound 1 hemifumarate.

FIG. 2 shows the DSC thermogram of Compound 1 hemifumarate.

FIG. 3 shows the TGA thermogram of Compound 1 hemifumarate.

FIG. 4 shows the XRPD diffractogram of Compound 1 dihydrochloride.

FIG. 5 shows the DSC thermogram of Compound 1 dihydrochloride.

FIG. 6 shows the TGA thermogram of Compound 1 dihydrochloride.

FIG. 7 shows the XRPD diffractogram of Compound 1 hydrochloride.

FIG. 8 shows the DSC thermogram of Compound 1 hydrochloride.

FIG. 9 shows the TGA thermogram of Compound 1 hydrochloride.

FIG. 10 shows the XRPD diffractogram of Compound 1 L-tartrate.

FIG. 11 shows the DSC thermogram of Compound 1 L-tartrate.

FIG. 12 shows the TGA thermogram of Compound 1 L-tartrate.

FIG. 13 shows the XRPD diffractogram of Compound 1 phosphate.

FIG. 14 shows the DSC thermogram of Compound 1 phosphate.

FIG. 15 shows the TGA thermogram of Compound 1 phosphate.

FIG. 16 shows the calculated XRPD diffractogram of Compound 1 hemifumarate hemimethanol.

FIG. 17 is an atomic displacement ellipsoid diagram of Compound 1 hemifumarate hemimethanol.

FIG. 18a is a packing diagram of Compound 1 hemifumarate hemimethanol along the crystallographic a axis.

FIG. 18b is a packing diagram of Compound 1 hemifumarate hemimethanol along the crystallographic b axis.

FIG. 18c is a packing diagram of Compound 1 hemifumarate hemimethanol along the crystallographic c axis.

### DETAILED DESCRIPTION

Provided herein are processes for preparing potent and selective KRAS inhibitors and pharmaceutically acceptable salts thereof. An example of such a compound is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 1*):

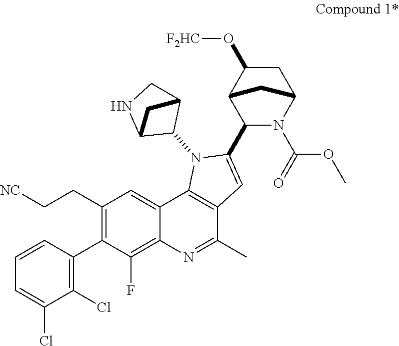

or pharmaceutically acceptable salts thereof, including its atropisomer methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 1), methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate salt (Compound 1-hemifumarate), and methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate hemimethanol salt (Compound 1-hemifumarate hemimethanol). This compound is useful for the treatment of KRAS-mediated diseases, including a variety of cancers, as disclosed in PCT Application No. PCT/US2024/025160 and U.S. patent application Ser. No. 18/639,041, the entire contents of which are incorporated herein by reference.

### I. Definitions

Listed below are definitions of various terms used to describe the processes provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the compound and its crystalline forms belong. Generally, the nomenclature used herein, and the laboratory procedures used in organic chemistry, and chemical manufacturing processes are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present disclosure also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, alcohols (e.g., MeOH, EtOH, iso-propanol or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66(1), 1-19, T. S. Wiedmann, et al., Asian J. Pharm. Sci., 2016; 11, 722-34. D. Gupta et al., Molecules, 2018, 23(7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011).

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and +0.1% from the specified value, as such variations are appropriate to perform the disclosed methods.

The expressions “ambient temperature” and “room temperature” are understood in the art, and refer generally to a temperature, e.g., a reaction temperature, which is about the temperature of the room in which the reaction is carried out, e.g., a temperature from about 20° C. to about 30° C.

At various places in the present specification, variables defining divalent linking groups may be described. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted,” unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl,” employed alone or in combination with other terms, refers to a straight-chain or branched hydrocarbon group corresponding to an alkyl group having one or more carbon-carbon triple bonds. The term “Cₙ₋ₘ alkynyl” refers to an alkynyl group having n to m carbon atoms. An alkynyl group formally corresponds to an alkyne with one C—H bond replaced by the point of attachment of the alkyl group to the remainder of the compound. In some embodiments, the alkynyl moiety contains 2 to 6 or 2 to 4 carbon atoms. Example alkynyl groups include, but are not limited to, ethynyl, propyn-1-yl, propyn-2-yl, and the like.

The term “alkoxy,” employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms. The term “Cₙ₋ₘ dialkoxy” refers to a linking group of formula —O—(Cₙ₋ₘ alkyl)-O—, the alkyl group of which has n to m carbons. Example dialkoxy groups include —OCH₂CH₂O— and OCH₂CH₂CH₂O—. In some embodiments, the two O atoms of a Cₙ₋ₘ dialkoxy group may be attached to the same B atom to form a 5- or 6-membered heterocycloalkyl group.

The terms “halo” or “halogen,” used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CC₃, CHCl₂, C₂Cl₅ and the like. In some embodiments, the haloalkyl group is a fluoroalkyl group.

The term “haloalkoxy,” employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

Preparation of compounds provided herein can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4^{th} Ed., (Wiley, 2006).

As used herein, the term “ester” refers to the replacement of the hydrogen of an acid by an alkyl group or another organic group. For example, esters are represented by —CO₂R, wherein R is a carbon atom of an organic group. Esters can also be in the form of boronic esters represented by —B(OR)₂, wherein R is a carbon atom of an organic group. Common boronic esters include allylboronic acid pinacol ester (also referred to as “pinacol ester”), phenyl boronic acid trimethylene glycol ester, and diisopropoxymethylborane.

Preparation of compounds provided herein can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., Marchs Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4^{th} Ed., (Wiley, 2006).

As used herein, “protecting group” refers to a molecular framework that is introduced onto a specific functional group in a poly-functional molecule to block its reactivity under reaction conditions needed to make modifications elsewhere in the molecule. As such, the phrase “nitrogen protecting group” refers to a protecting group as described above that protects or masks a nitrogen atom. The phrase “hydrolysable protecting group” refers to a protecting group as described above that can be hydrolyzed or cleaved under acidic or basic conditions.

In some embodiments, the protecting group is benzyloxycarbonyl (Cbz), 2,2,2-trichloroethoxycarbonyl (Troc), 2-(trimethylsilyl)ethoxycarbonyl (Teoc), 2-(4-trifluoromethylphenylsulfonyl)ethoxycarbonyl (Tsc), tert-butoxycarbonyl (Boc), 1-adamantyloxycarbonyl (Adoc), 2-adamantylcarbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBoc), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, methoxymethyl (MOM), 2-methoxyethoxymethyl (MEM), t-butoxymethyl (Bum), benzyloxymethyl (BOM), or 2-tetrahydropyranyl (THP). In some embodiments, the protecting group is methoxymethyl (MOM), 2-methoxyethoxymethyl (MEM), allyl, t-butyldimethylsilyl (TBDMS or TBS), or pivoyl (Piv). In some embodiments, the protecting group is 2-(trimethylsilyl)ethoxymethyl (SEM), or tosyl (Ts). In some embodiments, the protecting group is tert-butoxycarbonyl (Boc).

As used herein, the phrase “protecting group reagent” refers to a reactant that installs a protecting group on another reactant in a process. Protecting group reagents include reactants that protect a free nitrogen atom or free oxygen atom. Examples of protecting group reagents include but are not limited to MOMCl, MEMCl, Boc₂O, TrtCl, SEMCl, BnCl, PivCl, TBDPSCl, TIPSCl, TMSCl, and BzCl.

As used herein, the term “coupling agent” refers to a chemical species that aids in the formation of a carbon-carbon bond in a reaction between a species having a leaving group and a reactive species. Exemplary coupling reagents include, but are not limited to, a palladium catalyst such as tetrakis(triphenylphosphine)palladium(0), bis(di-tert-butyl)-dimethylaminophenylphosphone dichloride palladium (II) (Pd-132), bis(triphenylphosphine)palladium(II) dichloride, and palladium (II) acetate in combination with reagents such as n-Bu₄NOAc, Cs₂CO₃, piperidine, copper iodide, diethylamine, K₂CO₃, NiCl₂-glyme, NiBr₂-glyme, potassium t-butoxide, potassium phosphate, and KOH.

As used herein, the term “halogenating agent” refers to a reagent that installs a halo group as defined supra on a reactant. As such, a brominating agent installs a bromo group on a reactant and an iodinating agent installs an iodo group on a reactant. Examples of halogenating agents include, but are not limited to, elemental halogens, e.g., chorine, bromine, or iodine, interhalogen compounds, e.g., BrF₃, IF₅, ICl, and N-haloimides, e.g., NCS, NBS, or NIS.

As used herein, the phrase “reducing agent” refers to a chemical species that donates an electron or hydride in a redox reaction. Examples of reducing agents include, but are not limited to, NaBH₄, LiAlH₄, sodium hydride, Red-AI, sodium amalgam, diborane, hydrogen gas, Xantphos, Cu(OAc)₂, and polymethylhydrosiloxane (PMHS), alone or in combination.

As used herein, the phrase “alkylating reagent” refers to chemical species that installs an alkyl group as defined supra on a reactant. Examples of alkylating agents include, but are not limited to, haloalkanes, such as bromoalkanes and iodoalkanes, e.g., Mel, and alkyl sulfonate esters, such as alkyl methanesulfonates, alkyl arenesulfonates, or alkyl trifluoromethanesulfoneates.

As used herein, the phrase “carbonylating agent” refers to chemical species that installs a carbonyl (C═O) group on a reactant. Examples of carbonylating agents include, but are not limited to, phosgene, triphosgene, and 1,1′-carbonyldimidazole.

As used herein, the phrase “reducing agent” refers to a chemical species that donates an electron or hydride in a redox reaction. Examples of reducing agents include, but are not limited to, NaBH₄, LiAlH₄, sodium hydride, Red-AI, sodium amalgam, diborane, hydrogen gas, Xantphos, Cu(OAc)₂, and polymethylhydrosiloxane (PMHS), alone or in combination.

As used herein, the phrase “alkylating reagent” refers to chemical species that installs an alkyl group as defined supra on a reactant. Examples of alkylating agents include, but are not limited to, haloalkanes, such as bromoalkanes and iodoalkanes, e.g., Mel, and alkyl sulfonate esters, such as alkyl methanesulfonates, alkyl arenesulfonates, or alkyl trifluoromethanesulfoneates.

As used herein, the phrase “carbonylating agent” refers to chemical species that installs a carbonyl (C═O) group on a reactant. Examples of carbonylating agents include, but are not limited to, phosgene, triphosgene, and 1,1′-carbonyldimidazole.

The reactions of the processes described herein can be carried out at appropriate temperatures that can be readily determined by the skilled artisan. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures).

The reactions of the processes described herein can be carried out in air or under an inert atmosphere. Typically, reactions containing reagents or products that are substantially reactive with air can be carried out using air-sensitive synthetic techniques that are well known to the skilled artisan.

In some embodiments, preparation of compounds can involve the addition of acids or bases to effect, for example, catalysis of a desired reaction or formation of salt forms such as acid addition salts.

As used herein, the term “acid” refers to any species that can donate a proton or forming a covalent bond with an electron pair. Example acids can be inorganic or organic acids. Inorganic acids include HCl, hydrobromic acid, sulfuric acid, phosphoric acid, and nitric acid. Organic acids include formic acid, acetic acid, propionic acid, butanoic acid, benzoic acid, 4-nitrobenzoic acid, methanesulfonic acid, p-toluenesulfonic acid, benzenesulfonic acid, tartaric acid, trifluoroacetic acid, propiolic acid, butyric acid, 2-butynoic acid, vinyl acetic acid, pentanoic acid, hexanoic acid, heptanoic acid, octanoic acid, nonanoic acid and decanoic acid.

As used herein, the term “base” refers to any species that contains a filled orbital containing an electron pair which is not involved in bonding. Example bases include LiOH, NaOH, KOH, Li₂CO₃, Na₂CO₃, K₂CO₃, and Cs₂CO₃. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include sodium and potassium salts of methyl, ethyl, n-propyl, i-propyl, n-butyl, t-butyl, TMS and cyclohexyl substituted amides.

The following abbreviations may be used herein: AcOH (acetic acid); aq. (aqueous); br (broad); calc. (calc.); Cs₂CO₃ (cesium carbonate); d (doublet); dd (doublet of doublets); DCM (dichloromethane); DIPEA (N,N-diisopropylethylamine); DIBAL (diisobutylaluminium hydride); DMF (N,N-dimethylformamide); DMSO (dimethyl sulfoxide); Et (ethyl); EtOAc (ethyl acetate); EtOH (ethanol); eq. (equivalent(s)); g (gram(s)); h (hour(s)); HCl (hydrochloric acid); HPLC (high performance liquid chromatography); Hz (hertz); J (coupling constant); K₂CO₃ (potassium carbonate); KOH (potassium hydroxide); L (liter(s)); LCMS (liquid chromatography-mass spectrometry); Li₂CO₃ (lithium carbonate); LiOH (lithium hydroxide); m (multiplet); M (molar); MS (mass spectrometry); Me (methyl); MeCN (acetonitrile); MeOH (methanol); mg (milligram(s)); min. (minutes(s)); mL (milliliter(s)); mmol (millimole(s)); mol (mole(s)); MTBE (methyl tert-butyl ether); N (normal); NaBH₄ (sodium borohydride); NaBH₃CN (sodium cyanoborohydride); Na₂CO₃ (sodium carbonate); NaHCO₃ (sodium bicarbonate); NaOH (sodium hydroxide); NBS (N-bromosuccinimide); NCS (N-chlorosuccinimide); NIS (N-iodosuccinimide); NEt₃ (triethylamine); NLT (not less than); nM (nanomolar); NMP (N-methyl-2-pyrrolidinone); NMR (nuclear magnetic resonance spectroscopy); OTf (trifluoromethanesulfonate); Ph (phenyl); pM (picomolar); r.t. (room temperature), s (singlet); t (triplet or tertiary); tert (tertiary); tt (triplet of triplets); TFA (trifluoroacetic acid); THF (tetrahydrofuran); TMS (trimethylsilyl) wt % (weight percent). Brine is sat. aq. sodium chloride. In vacuo is under vacuum.

Compounds of the present disclosure can exist in the form of atropisomers (i.e., conformational diastereoisomers) that can be stable at ambient temperature and separable, e.g., by chromatography. For example, compounds provided herein can exist in the form of atropisomers in which the conformation of the dichlorophenyl relative to the remainder of the molecule is as shown by the partial formulae Formula (II-A) or Formula (II-B) below. Reference to the compounds described herein or any of the embodiments is understood to include all such atropisomeric forms of the compounds, including, without limitation, the atropisomeric forms represented by Formula (II-A) or Formula (II-B) below. The asymmetry of atropisomers is assigned as either Rₐ or Sₐ, as determined by conventional methods of characterizing points of asymmetry.

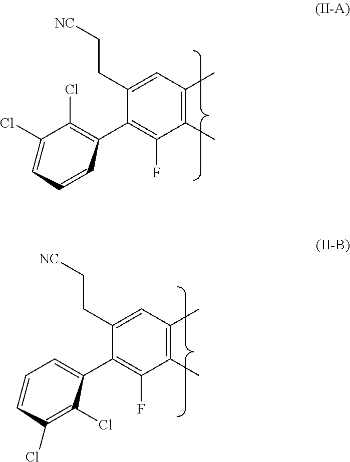

For example, Compound 1* can exist as two atropisomers that are stable at ambient temperature, methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 1) and methyl (1R,3R,4R,5S)-3-((S,)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate.

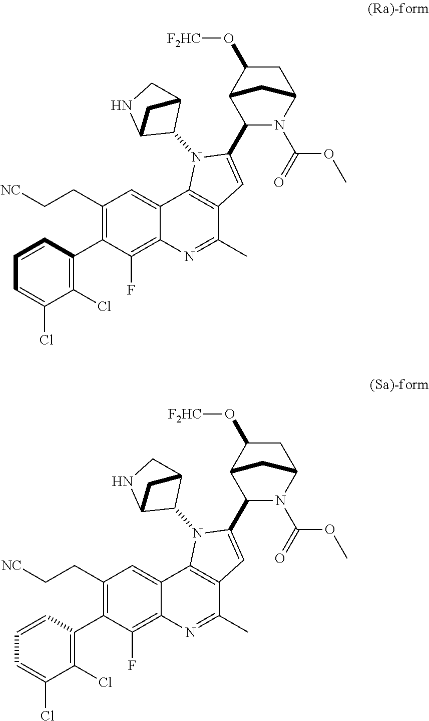

An aspect of the present disclosure relates to methods for the preparation of compounds of Formula I, including Compound 1*, and intermediates used for the preparation of compounds of Formula I, including Compound 1*, wherein one atropisomer is in excess compared to the other isomer. The term “atropisomeric purity” refers to the percentage of a given atropisomer present in the compound compared to the total amount of the compound. In some embodiments, the atropisomeric purity of each of the atropisomeric compounds described herein can be greater than 50%, such as about 80% or greater, about 90% or greater, about 95% or greater, about 96% or greater, about 97% or greater, about 98% or greater, about 99% or greater, about 99.5% or greater, or about 99.9% or greater. When the enantiomeric purity of an atropisomeric compound is about 95% or greater, about 96% or greater, about 97% or greater, about 98% or greater, about 99% or greater, about 99.5% or greater, or about 99.9% or greater, the atropisomeric compound can be referred to as being “substantially free” of the alternative atropisomer.

### II. Processes

In an aspect, provided herein is a process for preparing a compound of Formula I:

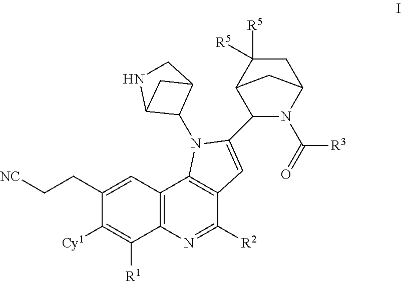

wherein
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen;
   R² is C₁₋₃ alkyl optionally substituted with OH;
   R³ is OR^{3A};
   R^{3A} is selected from C₁₋₃ alkyl, C₂₋₃ alkenyl, and C₂₋₃ alkynyl;
each R⁵ is independently selected from H, D, halo, C₁₋₃ alkyl, OR^{a5}, C₁₋₃ haloalkyl, C₂₋₃ alkenyl, and C₂₋₃ alkynyl; and
   each R^{a5} is independently selected from H, C₁₋₃ alkyl, and C₁₋₃ haloalkyl;
   comprising deprotecting a compound of Formula II:

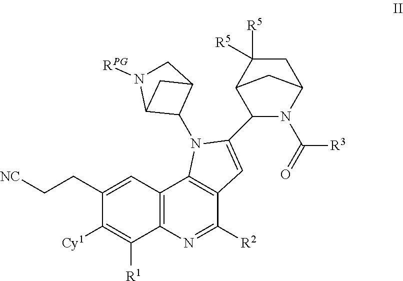

wherein
   R^{PG} is a nitrogen protecting group;
   to produce the compound of Formula I.

In some embodiments, R² is methyl.

In some embodiments, Cy¹ is 2,3-dichlorophenyl.

In some embodiments, R³ is OCH₃.

In some embodiments, one R⁵ is H. In some embodiments, one R⁵ is OCHF₂.

In some embodiments, R^{PG} is a hydrolysable protecting group and the deprotecting comprises hydrolyzing the compound of Formula II. In other embodiments, R^{PG} is benzyloxycarbonyl (Cbz), 2,2,2-trichloroethoxycarbonyl (Troc), 2-(trimethylsilyl)ethoxycarbonyl (Teoc), tert-butoxycarbonyl (Boc), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, benzyloxymethyl (BOM), or 2-tetrahydropyranyl (THP).

In some embodiments of the processes herein, R^{PG} is tert-butyloxycarbonyl. In other embodiments, the deprotecting comprises reacting the compound of Formula II with an acid. In some embodiments, the deprotecting comprises reacting the compound of Formula II with a Lewis acid. In yet other embodiments, the acid is HCl. In other embodiments, the acid is a trialkylsilyl halide, for example TMSI (trimethylsilyl iodide).

In some embodiments, the reaction can be carried out in the presence of a hydroxylic solvent or mixtures thereof, e.g., water, MeOH or EtOH. In some embodiments, the reaction can be carried out in the presence of a halogenated solvent or mixtures thereof, e.g., DCM, chloroform, 1,2-dichloroethane, or 1,1,1-trichloroethane. In some embodiments, the reaction can be carried out in more than one stage, e.g., treatment with a Lewis acid in the presence of a halogenated solvent, followed by addition of a hydroxylic solvent.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at r.t.

In another aspect, provided herein is a process for preparing a compound of Formula II:

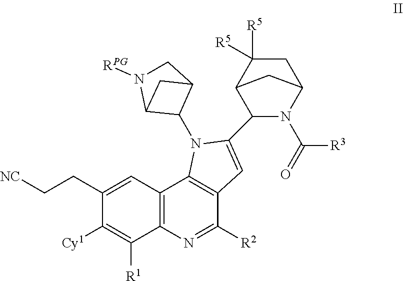

wherein
   R^{PG} is a nitrogen protecting group;
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen;
   R² is C₁₋₃ alkyl optionally substituted with OH;
   R³ is OR^{3A};
   R^{3A} is selected from C₁₋₃ alkyl, C₂₋₃ alkenyl, and C₂₋₃ alkynyl;
   each R⁵ is independently selected from H, D, halo, C₁₋₃ alkyl, OR^{a5}, C₁₋₃ haloalkyl, C₂₋₃ alkenyl, and C₂₋₃ alkynyl; and
   each R^{a5} is independently selected from H, C₁₋₃ alkyl, and C₁₋₃ haloalkyl;
   comprising cyclizing a compound of Formula III:

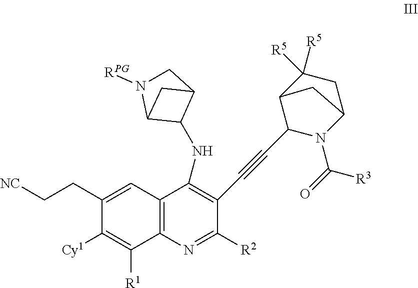

to form the compound of Formula II.

In some embodiments, R² is methyl.

In some embodiments, Cy¹ is 2,3-dichlorophenyl.

In some embodiments, R³ is OCH₃.

In some embodiments, one R⁵ is H. In some embodiments, one R⁵ is OCHF₂.

In other embodiments, R^{PG} is benzyloxycarbonyl (Cbz), 2,2,2-trichloroethoxycarbonyl (Troc), 2-(trimethylsilyl)ethoxycarbonyl (Teoc), tert-butoxycarbonyl (Boc), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, benzyloxymethyl (BOM), or 2-tetrahydropyranyl (THP).

In some embodiments of the processes herein, R^{PG} is tert-butyloxycarbonyl.

In some embodiments, the cyclizing comprises reacting the compound of Formula III with a base. Examples of suitable bases include alkali metal carbonates, e.g., K₂CO₃ or Cs₂CO₃.

In some embodiments, the reaction can be carried out in the presence of a polar aprotic solvent or mixtures thereof, e.g., THF, 1,4-dioxane, MeCN, DMF, DMSO or NMP.

In some embodiments, the reaction can be carried out at a temperature in the range from about 0° C. to about 150° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., about 120° C., about 130° C., about 140° C., about 150° C. In some embodiments, the reaction can be carried out at a temperature from about 80° C. to about 90° C., or from about 80° C. to about 85° C.

In yet another aspect, provided herein is a process of preparing a compound of Formula III:

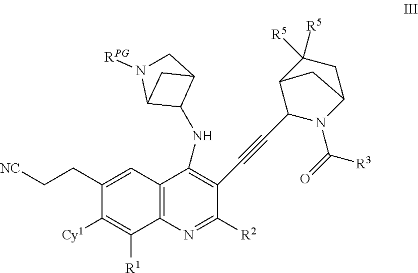

comprising
   coupling a compound of Formula IV:

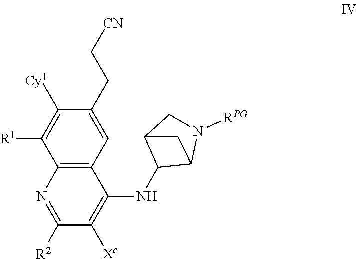

with a compound of Formula V:

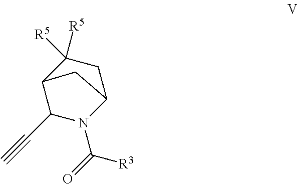

to form the compound of Formula III
wherein
X^{c} is Cl, Br, or I;
R^{PG} is a nitrogen protecting group;
Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
R¹ is halogen;
R² is C₁₋₃ alkyl optionally substituted with OH;
R³ is OR^{3A};
R^{3A} is selected from C₁₋₃ alkyl, C₂₋₃ alkenyl, and C₂₋₃ alkynyl;
each R⁵ is independently selected from H, D, halo, C₁₋₃ alkyl, OR^{a}s, C₁₋₃ haloalkyl, C₂₋₃ alkenyl, and C₂₋₃ alkynyl; and
each R^{a5} is independently selected from H, C₁₋₃ alkyl, and C₁₋₃ haloalkyl.

In some embodiments, X^{c} is Cl. In some embodiments, X^{c} is Br. In some embodiments, X^{c} is I.

In some embodiments, R² is methyl.

In some embodiments, Cy¹ is 2,3-dichlorophenyl.

In some embodiments, R³ is OCH₃.

In some embodiments, one R⁵ is H. In some embodiments, one R⁵ is OCHF₂.

In some embodiments, R^{PG} is tert-butyloxycarbonyl.

In other embodiments, R^{PG} is benzyloxycarbonyl (Cbz), 2,2,2-trichloroethoxycarbonyl (Troc), 2-(trimethylsilyl)ethoxycarbonyl (Teoc), tert-butoxycarbonyl (Boc), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, benzyloxymethyl (BOM), or 2-tetrahydropyranyl (THP).

The coupling can be carried out in the presence of a palladium catalyst. Suitable palladium catalysts can include palladium(0) catalysts, e.g., Pd₂(dba)₃ or Pd(PPh₃)₄ and palladium(II) catalysts, e.g., Pd(PPh₃)₂Cl₂, Pd(dppe)Cl₂, Pd(dppp)Cl₂, or Pd(dppf)Cl₂. Palladium(II) catalysts also include, e.g., palladium (II) acetate. In some embodiments, the reaction can optionally be carried out in the presence of a cocatalyst, such as a copper cocatalyst, e.g., a copper halide, such as CuI, but the reaction can also be carried out under copper-free conditions. The coupling can be carried out in the presence of a base. Suitable bases can include amine bases, e.g., NEt₃ or DIPEA. Other suitable bases include alkali metal carbonates, e.g., K₂CO₃ or Cs₂CO₃. Other suitable bases include carboxylic acid salts such as acetate salts, e.g., NaOAc or n-Bu₄NOAc.

The coupling can be carried out in the presence of a phosphine reagent. Suitable phosphine reagents can include tris (4-fluorophenyl)phosphine and triphenylphosphine.

In some embodiments, the reaction can be carried out in the presence of a polar aprotic solvent or mixtures thereof, e.g., THF, 1,4-dioxane, MeCN, DMF, or NMP. Polar, aprotic solvents also include, e.g., DMSO.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., about 100° C. In some embodiments, the reaction can be carried out at a temperature from about 60° C. to about 80° C., such as about 80° C.

In an embodiment, the process further comprises preparing the compound of Formula IV:

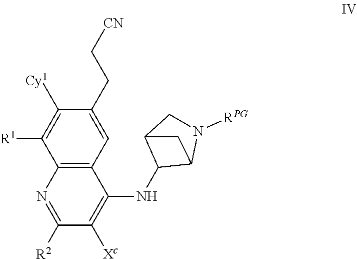

wherein
   X^{c} is Cl, Br, or I;
   R^{PG} is a nitrogen protecting group;
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen; and
   R² is C₁₋₃ alkyl optionally substituted with OH;
   comprising halogenating a compound of Formula VI:

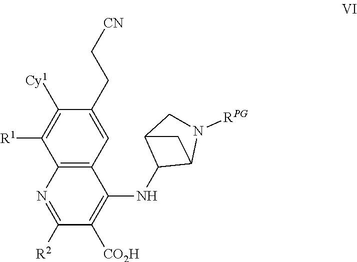

to form the compound of Formula IV.

In some embodiments, X^{c} is Cl. In some embodiments, X^{c} is Br. In some embodiments, X^{c} is I.

In some embodiments, R² is methyl.

In some embodiments, Cy¹ is 2,3-dichlorophenyl.

In some embodiments, R^{PG} is tert-butyloxycarbonyl.

In some embodiments, the halogenating comprises reacting the compound of Formula IV with a halogenating agent.

In some embodiments, the halogenating is chlorinating, and the halogenating agent is a chlorinating agent, e.g., NCS. In some embodiments the halogenating is brominating, and the halogenating agent is a brominating agent, e.g., NBS. In some embodiments, the halogenating is iodinating, and the halogenating agent is an iodinating agent, e.g., NIS.

In some embodiments, the halogenating comprises reacting the compound of Formula IV with an iodinating agent. An example of a suitable iodinating agent is N-iodosuccinimide.

The halogenating can be carried out in the presence of a base. In still other embodiments, the base is a phosphate base. In some embodiments, the base is trisodium phosphate.

In some embodiments, the reaction can be carried out in the presence of a polar aprotic solvent or mixtures thereof, e.g., THF, 1,4-dioxane, or MeCN.

In some embodiments, the reaction can be carried out at a temperature in the range from about 0° C. to about 50° C., such as a temperature of about 5° C., about 10° C., about 15° C., about 20° C., about 25° C., about 30° C., about 40° C., or about 50° C. In some embodiments, the reaction can be carried out at a temperature from about 20° C. to about 30° C. In some embodiments, the reaction can be carried out at about r.t.

In an embodiment, the process further comprises preparing the compound of

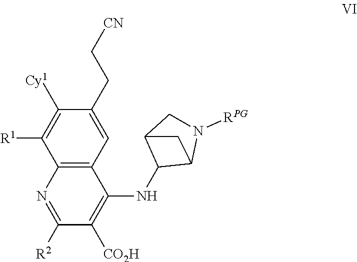

comprising hydrolyzing a compound of Formula VII:

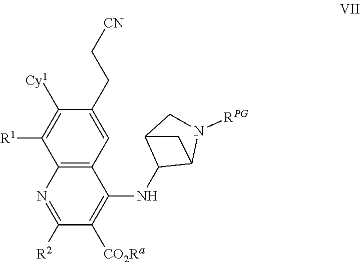

wherein
R^{a} is C₁₋₃ alkyl;
R^{PG} is a nitrogen protecting group;
Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
R¹ is halogen; and
R² is C₁₋₃ alkyl optionally substituted with OH;
to form the compound of Formula VI.

In some embodiments, X^{c} is Cl. In some embodiments, X^{c} is Br. In some embodiments, X^{c} is I.

In some embodiments, R² is methyl.

In some embodiments, Cy¹ is 2,3-dichlorophenyl.

In some embodiments, R^{PG} is tert-butyloxycarbonyl.

In some embodiments, R^{a} is ethyl.

In some embodiments, the hydrolyzing is carried out by reacting the compound of Formula VI in the presence of a base. Examples of suitable bases include alkali metal carbonate bases such as K₂CO₃ or Cs₂CO₃. Other examples of suitable bases include alkali or alkaline earth hydroxide bases such as NaOH or KOH. Further examples of suitable bases include alkali metal trialkylsiloxide bases such as NaOTMS or KOTMS. In other embodiments, the base is NaOTMS. In yet other embodiments, the base is NaOH.

In some embodiments, the reaction can be carried out in the presence of a hydroxylic solvent or mixtures thereof, e.g., water, MeOH or EtOH. In some embodiments, the reaction can be carried out in the presence of water. In some embodiments, the reaction can be carried out in the presence of water and one or more water-miscible co-solvents, e.g., THF, 1,4-dioxane, MeCN, MeOH or EtOH.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., about 100° C. In some embodiments, the reaction can be carried out at a temperature from about 40° C. to about 60° C., such as about 50° C.

In an embodiment, the process further comprises preparing the compound of Formula VII:

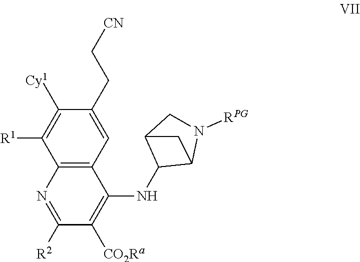

wherein
   R^{a} is C₁₋₃ alkyl;
   R^{PG} is a nitrogen protecting group;
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen; and
   R² is C₁₋₃ alkyl optionally substituted with OH;
   comprising reacting a compound of Formula VIII;

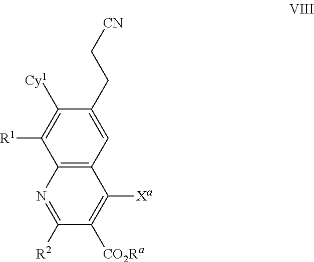

wherein
   X^{a} is halo or OH;
   with a compound of Formula IX:

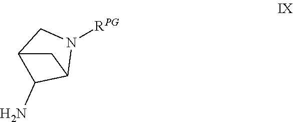

wherein
   R^{PG} is a nitrogen protecting group;
   to form the compound of Formula VII.

In some embodiments, R² is methyl.

In some embodiments, Cy¹ is 2,3-dichlorophenyl.

In some embodiments, R^{PG} is tert-butyloxycarbonyl.

In some embodiments, R^{a} is ethyl.

In some embodiments, X^{a} is Cl or OH. In some embodiments, X^{a} is Cl. In some embodiments, X^{a} is OH.

In other embodiments, R^{PG} is benzyloxycarbonyl (Cbz), 2,2,2-trichloroethoxycarbonyl (Troc), 2-(trimethylsilyl)ethoxycarbonyl (Teoc), tert-butoxycarbonyl (Boc), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, benzyloxymethyl (BOM), or 2-tetrahydropyranyl (THP).

In some embodiments, the reacting is performed in the presence of a base. In other embodiments, the base is a trialkylamine base such as NEt₃ or DIPEA. In some embodiments, the reacting is performed in the absence of a base.

In some embodiments, the compound of Formula IX is a salt thereof. In some embodiments, the compound of Formula IX is an oxalate salt.

In some embodiments, the reaction is carried out in the presence of a lithium salt, e.g., LiCl or Li₂CO₃.

In some embodiments, the reaction can be carried out in the presence of a polar aprotic solvent or mixtures thereof, e.g., THF, 1,4-dioxane, MeCN, DMF, DMSO or NMP.

In some embodiments, the reaction can be carried out at a temperature in the range from about 0° C. to about 150° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., about 120° C., about 130° C., about 140° C., about 150° C. In some embodiments, the reaction can be carried out at a temperature from about 70° C. to about 90° C., or at about 80° C.

In an embodiment, the process further comprises preparing the compound of Formula VIII:

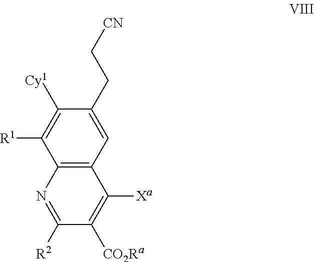

wherein
   R^{a} is C₁₋₃ alkyl;
   X^{a} is halo or OH;
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen; and
   R² is C₁₋₃ alkyl optionally substituted with OH;
   comprising reducing a compound of Formula X:

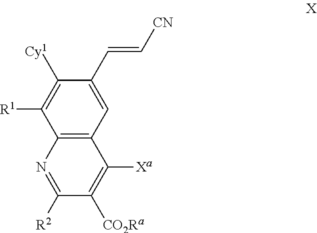

to form the compound of Formula VIII.

In some embodiments, R² is methyl.

In some embodiments, Cy¹ is 2,3-dichlorophenyl.

In some embodiments, R^{a} is ethyl.

In some embodiments, X^{a} is Cl or OH. In some embodiments, X^{a} is Cl. In some embodiments, X^{a} is OH.

In some embodiments, the reducing comprises reacting the compound of Formula X with a reducing agent. The reducing agent can be a silane reducing agent such as polymethylhydrosiloxane (PMHS). The reaction can be carried out in the presence of a copper catalyst formed by a suitable copper (II) salt such as Cu(OAc)₂ and a suitable ligand such as Xantphos or DPEphos. In other embodiments the reducing agent can be a borohydride such as NaBH₄ or NaBH₃CN.

In some embodiments, the reaction can be carried out in the presence of suitable solvent or mixtures thereof, e.g., toluene, tert-butanol, THF, 1,4-dioxane, MeCN or pyridine.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 40° C. to about 70° C., from about 50° C. to about 60° C. or at about 50° C. or about 60° C.

In an embodiment, the process further comprises preparing the compound of Formula X-A:

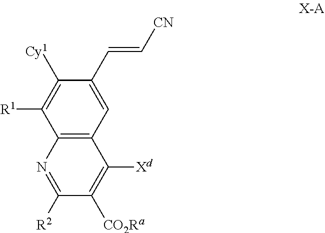

wherein
   R^{a} is C₁₋₃ alkyl;
   X^{a} is halo or OH;
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen; and
   R² is C₁₋₃ alkyl optionally substituted with OH;
   comprising halodehydroxylating a compound of Formula XI:

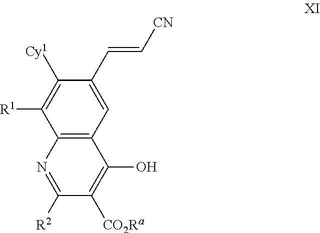

to form the compound of Formula X.

In some embodiments, R² is methyl.

In some embodiments, Cy¹ is 2,3-dichlorophenyl.

In some embodiments, R^{a} is ethyl.

In some embodiments, X^{d} is Cl, Br, or I. In some embodiments, X^{d} is Cl.

In some embodiments, the halodehydroxylating comprises chlorodehydroxylating.

The halodehydroxylating can be performed in the presence of suitable halodehydroxylating (or chlorodehydroxylating agent) such as phosphoryl chloride (POCl₃) or thionyl chloride. In yet other embodiments, the halodehydroxylating is performed in the presence of a suitable catalyst such as benzyltriethylammonium chloride (BTEAC).

In some embodiments, the reaction can be carried out in the presence of suitable solvent or mixtures thereof, e.g., toluene, THF, 1,4-dioxane, N,N-diethylaniline, or MeCN.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 10° C. to about 70° C., such as from about 20° C. to about 60° C.

In an embodiment, the process further comprises preparing the compound of Formula XI:

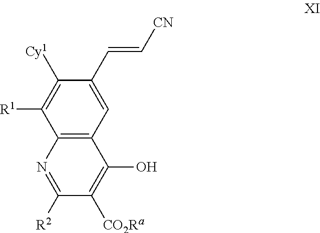

wherein
   R^{a} is C₁₋₃ alkyl;
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen; and
   R² is C₁₋₃ alkyl optionally substituted with OH;
   comprising coupling a compound of Formula XII:

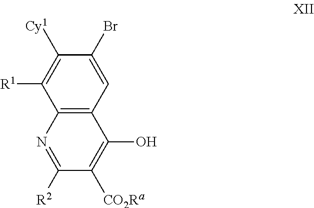

with a C₂₋₄ alkene that is optionally substituted with CN to form the compound of Formula XI.

In some embodiments, R² is methyl.

In some embodiments, Cy¹ is 2,3-dichlorophenyl.

In some embodiments, R^{a} is ethyl.

In some embodiments, the C₂₋₄ alkene that is optionally substituted with CN is acrylonitrile.

The coupling can be performed in the presence of a catalyst. The catalyst can be a palladium catalyst, such as a palladium(0) or palladium(II) catalyst. In yet other embodiments, the palladium catalyst is bis(di-tert-butyl)-(4-dimethylaminophenyl)phosphine)dichloridopalladium (II) (Pd-132) (Pd-132).

In some embodiments, the reaction can be carried out in the presence of suitable solvent or mixtures thereof, e.g., toluene, THF, 1,4-dioxane, N,N-diethylaniline, or MeCN.

In some embodiments, the reaction can be carried out at a temperature in the range from about 0° C. to about 120° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., or about 120° C. In some embodiments, the reaction can be carried out at a temperature from about 70° C. to about 100° C., or from about 80° C. to about 90° C., such as at about 85° C.

In an embodiment, the process further comprises preparing the compound of Formula XII:

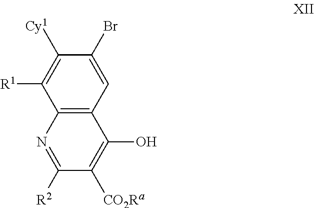

wherein
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen; and
   R² is C₁₋₃ alkyl optionally substituted with OH;
   comprising reacting a compound of Formula XIII:

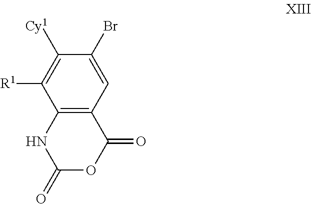

with a compound of Formula XIV:

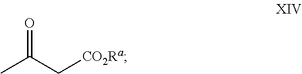

wherein R^{a} is C₁₋₃ alkyl;
to form the compound of Formula XII.

In some embodiments, Cy¹ is 2,3-dichlorophenyl.

In some embodiments, R^{a} is ethyl.

In some embodiments, the reacting is carried out in the presence of a base. Suitable bases include carboxylic acid salts, such as acetate salts. Example of suitable carboxylic acid salts include ammonium, tetraalkylammonium or alkali metal salts. In some embodiments the base is an alkali metal acetate salts such as sodium acetate or potassium acetate.

In some embodiments, the reaction can be carried out in the presence of suitable solvent or mixtures thereof, e.g., toluene, xylene or DMSO.

In some embodiments, the reaction can be carried out at a temperature in the range from about 0° C. to about 150° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., about 120° C., about 130° C., about 140° C., or about 150° C. In some embodiments, the reaction can be carried out at a temperature from about 50° C. to about 120° C., or from about 50° C. to about 100° C.

In an embodiment, the process further comprises preparing the compound of Formula XII:

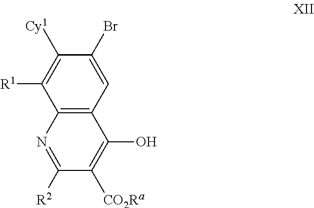

wherein
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen; and
   R² is C₁₋₃ alkyl optionally substituted with OH;
   comprising reacting a compound of Formula XIII:

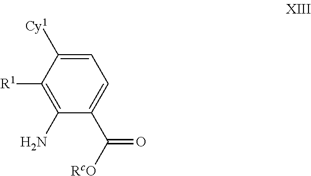

wherein R^{c} is C₁₋₃ alkyl;
with a compound of Formula XIV:

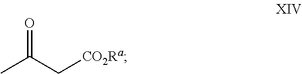

wherein R^{a} is C₁₋₃ alkyl;
to form the compound of Formula XII.

In some embodiments, Cy¹ is 2,3-dichlorophenyl.

In some embodiments, R^{a} is ethyl.

In some embodiments, R^{c} is methyl.

In some embodiments, the reacting is carried out in the presence of a base. Suitable bases include metal alkoxides. Example of suitable alkoxides include methoxide and ethoxide. In some embodiments the base is metal alkoxide such as sodium ethoxide, sodium methoxide, and potassium methoxide.

In some embodiments, the reaction can be carried out in the presence of suitable solvent or mixtures thereof, e.g., toluene, xylene or DMSO.

In some embodiments, the reaction can be carried out at a temperature in the range from about 0° C. to about 150° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., about 120° C., about 130° C., about 140° C., or about 150° C. In some embodiments, the reaction can be carried out at a temperature from about 50° C. to about 120° C., or from about 50° C. to about 100° C. In some embodiments, the reaction can be carried out at reflux.

In an embodiment, the process further comprises preparing the compound of Formula XIII:

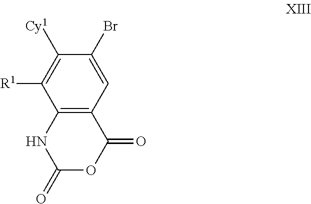

wherein
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen;
   comprising carbonylating a compound of Formula XV:

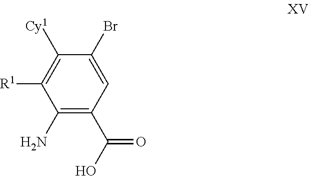

to form the compound of Formula XIII.

In some embodiments, Cy¹ is 2,3-dichlorophenyl.

In some embodiments, the carbonylating comprises reacting the compound of Formula XV with a carbonylating agent such as triphosgene.

In some embodiments, the reaction can be carried out in the presence of suitable solvent or mixtures thereof. Suitable solvents can include polar aprotic solvents and mixtures thereof, e.g., THF, 1,4-dioxane, or MeCN.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 50° C. to about 70° C., such as at about 60° C.

In an embodiment, the process further comprises preparing the compound of Formula XV:

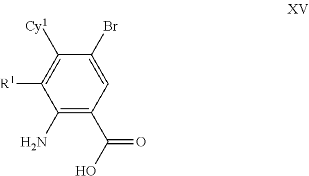

wherein
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen; and
   comprising hydrolyzing a compound of Formula XVI:

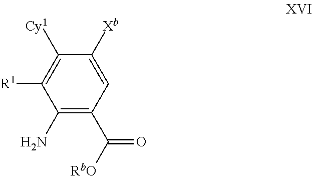

wherein
   X^{b} is Cl, Br, or I;
   R^{b} is C₁₋₃ alkyl;
to form the compound of Formula XV.

In some embodiments, Cy¹ is 2,3-dichlorophenyl.

In some embodiments, R^{b} is methyl.

In some embodiments, X^{b} is Br.

In some embodiments, the hydrolyzing comprises reacting the compound of Formula XVI in the presence of a base. Examples of suitable bases include alkali metal carbonate bases such as K₂CO₃ or Cs₂CO₃. Other examples of suitable bases include alkali or alkaline earth hydroxide bases such as NaOH or KOH. Further examples of suitable bases include alkali metal trialkylsiloxide bases such as NaOTMS or KOTMS. In other embodiments, the base is NaOTMS. In yet other embodiments, the base is NaOH.

In some embodiments, the reaction can be carried out in the presence of a hydroxylic solvent or mixtures thereof, e.g., water, MeOH or EtOH. In some embodiments, the reaction can be carried out in the presence of water. In some embodiments, the reaction can be carried out in the presence of water and one or more water-miscible co-solvents, e.g., THF, 1,4-dioxane, MeCN, MeOH or EtOH.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., about 100° C. In some embodiments, the reaction can be carried out at a temperature from about 40° C. to about 60° C., such as about 50° C.

In an embodiment, the process further comprises preparing the compound of Formula XVI:

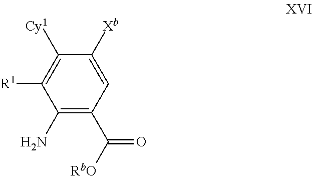

wherein
   X^{b} is Cl, Br, or I;
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen; and
   R^{b} is C₁₋₃ alkyl;
   comprising halogenating a compound of Formula XVII:

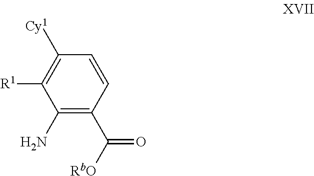

to form the compound of Formula XVI.

In some embodiments, Cy¹ is 2,3-dichlorophenyl.

In some embodiments, R^{b} is methyl.

In some embodiments, X^{b} is Br.

In some embodiments, the halogenating comprises reacting the compound of Formula VII with a halogenating agent. In other embodiments, the halogenating is brominating. In yet other embodiments, the halogenating agent is a brominating agent. In still other embodiments, the halogenating agent is N-bromosuccinimide (NBS).

In some embodiments, the reaction can be carried out in the presence of suitable solvent or mixtures thereof. Suitable solvents can include polar aprotic solvents and mixtures thereof, e.g., THF, 1,4-dioxane, or MeCN.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 40° C. to about 80° C., or from about 50° C. to about 70° C. such as at about 60° C.

In an embodiment, the process further comprises preparing the compound of Formula XVII:

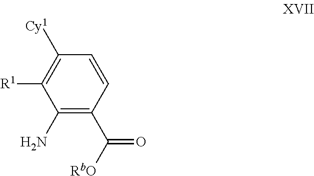

wherein
   R^{b} is C₁₋₃ alkyl;
   Cy¹ is phenyl optionally substituted with 1, 2, 3, or 4 substituents each selected from C₁₋₃ alkyl and halo;
   R¹ is halogen;
   comprising coupling a compound of Formula XVIII:

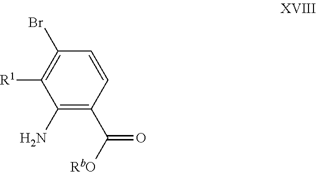

with a compound of Formula XIX:

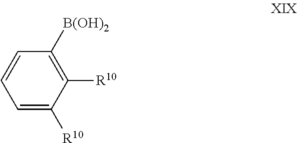

or an ester thereof;
wherein
   each R¹⁰ is independently selected from C₁₋₃ alkyl and halo;
in the presence of a palladium catalyst to form the compound of Formula XVII.

In some embodiments, Cy¹ is 2,3-dichlorophenyl.

In some embodiments, R^{b} is methyl.

In some embodiments, each R¹⁰ is Cl.

In some embodiments, the coupling is performed in the presence of a catalyst. The catalyst can a palladium catalyst, such as a palladium(0) or palladium(II) catalyst. In yet other embodiments, the palladium catalyst is bis(di-tert-butyl(4-dimethylaminophenyl)phosphine)dichloropalladium(II) (Pd-132).

In some embodiments, the reaction can be carried out in the presence of a suitable base. In some embodiments, the base is an alkali metal carbonate base e.g., K₂CO₃ or Cs₂CO₃. In some embodiments, the base is an alkali metal fluoride base, e.g., KF or CsF.

In some embodiments, the reaction can be carried out in the presence of suitable solvent or mixtures thereof, e.g., THF, 1,4-dioxane, or MeCN.

In some embodiments, the reaction can be carried out at a temperature in the range from about 0° C. to about 120° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., or about 120° C. In some embodiments, the reaction can be carried out at a temperature from about 50° C. to about 90° C., or from about 60° C. to about 80° C., such as at about 70° C.

In an embodiment, the process further comprises preparing the compound of Formula XVIII:

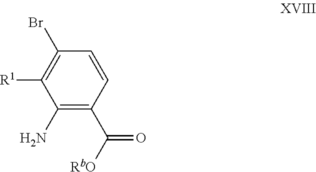

wherein
   R^{b} is C₁₋₃ alkyl;
   R¹ is halogen
   comprising esterifying a compound of Formula XX:

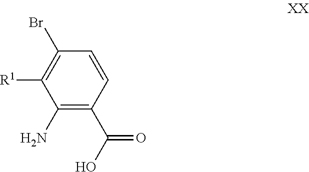

to form the compound of Formula XVIII.

In some embodiments, R^{b} is methyl.

In some embodiments, the esterifying comprises reacting the compound of Formula XX with an alkylating agent. In some embodiments, the alkylating agent is (R^{b})₂SO₄, wherein R^{b} is C₁₋₃ alkyl. In yet other embodiments, the alkylating agent is dimethyl sulfate.

In still other embodiments, the reacting is performed in the presence of a base. In some embodiments, the base is an alkali metal carbonate base e.g., K₂CO₃ or Cs₂CO₃.

In some embodiments, the reaction can be carried out in the presence of suitable solvent or mixtures thereof, e.g., THF, 1,4-dioxane, or MeCN.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 0° C. to about 50° C., or from about 5° C. to about 50° C., such as at about 20° C.

In an aspect, provided herein is a process of preparing a compound of Formula I′:

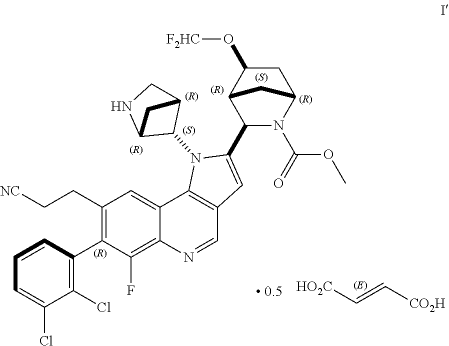

comprising reacting a compound of Formula I:

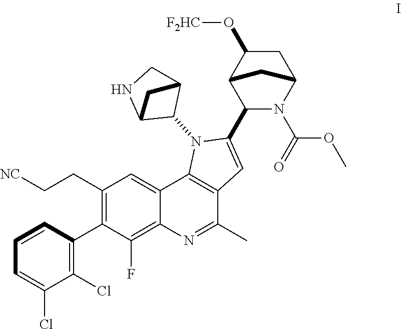

with fumaric acid to produce a compound of Formula I′.

In an aspect, provided herein is a process of preparing a compound of Formula I″:

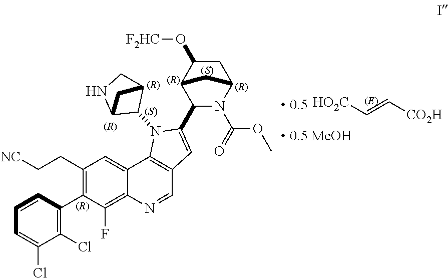

comprising reacting a compound of Formula I:

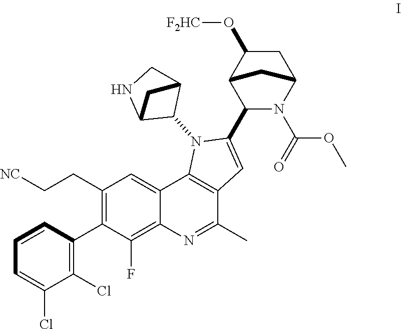

with fumaric acid and methanol to produce a compound of Formula I″.

In some embodiments of the processes herein, R¹ is fluoro.

In some embodiments of the processes herein, R² is C₁₋₃ alkyl.

In some embodiments of the processes herein, R² is methyl.

In some embodiments of the processes herein, R^{3A} is C₁₋₃ alkyl.

In some embodiments of the processes herein, R³ is OCH₃.

In some embodiments of the processes herein, Cy¹ is 2,3-dichlorophenyl.

In some embodiments, one R⁵ is H. In some embodiments, one R⁵ is OR^{a5}.

In some embodiments, R^{a5} is C₁₋₃ haloalkyl.

In some embodiments, R^{a5} is C₁₋₃ fluoroalkyl.

In some embodiments, one R⁵ is OCHF₂.

In some embodiments of the processes herein, R^{PG} is tert-butyloxycarbonyl.

In other embodiments of the processes herein, R^{PG} is benzyloxycarbonyl (Cbz), 2,2,2-trichloroethoxycarbonyl (Troc), 2-(trimethylsilyl)ethoxycarbonyl (Teoc), tert-butoxycarbonyl (Boc), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, benzyloxymethyl (BOM), or 2-tetrahydropyranyl (THP).

In some embodiments of the processes herein, X^{c} is I.

In some embodiments of the processes herein, the compound of Formula I is methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt, hydrate, of solvate thereof. In still other embodiments, the compound of Formula I is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 1*), or a pharmaceutically acceptable salt thereof. In some embodiments, the compound of Formula I is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 1). In some embodiments, the compound of Formula I is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate salt (Compound 1-hemifumarate). In some embodiments, the compound of Formula I is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate hemimethanol salt (Compound 1-hemifumarate hemimethanol). In some embodiments, the compound of Formula I is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate salt (Compound 1*-hemifumarate) or methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate hemimethanol salt (Compound 1*-hemifumarate hemimethanol. In some embodiments, the compound of Formula I is methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate.

In some embodiments of the processes herein, the compound of Formula II is tert-butyl 5-(8-(2-cyanoethyl)-7-(2,3-dichlorophenyl)-2-(5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate. In still other embodiments, the compound of Formula II is tert-butyl (1R,4R,5S)-5-(8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate (Compound 2*). In some embodiments, the compound of Formula II is tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate (Compound 2). In other embodiments, the compound of Formula II is tert-butyl (1R,4R,5S)-5-((Sₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate.

In some embodiments of the processes herein, the compound of Formula III is tert-butyl 5-((6-(2-cyanoethyl)-7-(2,3-dichlorophenyl)-3-((5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate. In still other embodiments, the compound of Formula III is tert-butyl (1R,4R,5S)-5-((6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate (Compound 3*). In some embodiments, the compound of Formula III is tert-butyl (1R,4R,5S)-5-(((Rₐ)-6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate (Compound 3). In other embodiments, the compound of Formula III is tert-butyl (1R,4R,5S)-5-(((Sₐ)-6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate.

In some embodiments of the processes herein, the compound of Formula IV is tert-butyl 5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate. In still other embodiments, the compound of Formula IV is tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (Compound 4*). In some embodiments, the compound of Formula IV is tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (Compound 4). In other embodiments, the compound of Formula IV is tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate.

In some embodiments of the processes herein, the compound of Formula V is methyl 5-(difluoromethoxy)-3-ethynyl-2-azabicyclo[2.2.1]heptane-2-carboxylate. In some embodiments of the processes herein, the compound of Formula V is methyl (1R,3R,4R,5S)-5-(difluoromethoxy)-3-ethynyl-2-azabicyclo[2.2.1]heptane-2-carboxylate (Compound 5).

In some embodiments of the processes herein, the compound of Formula VI is 4-((2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid. In still other embodiments, the compound of Formula VI is 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (Compound 6*). In some embodiments of the processes herein, the compound of Formula VI is (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (Compound 6). In other embodiments, the compound of Formula VI is (Sₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid.

In some embodiments of the processes herein, the compound of Formula VII is tert-butyl 5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate. In still other embodiments, the compound of Formula VII is tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (Compound 7*). In some embodiments, the compound of Formula VII is tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (Compound 7). In other embodiments, the compound of Formula VII is tert-butyl (1R,4R,5S)-5-(((Sₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate.

In some embodiments of the processes herein, the compound of Formula VIII is ethyl 4-chloro-6-(2-cyanoethyl)-7-(2,3-dichlorophenyl)-8-fluoro-2-methylquinoline-3-carboxylate (Compound 8a*). In still other embodiments, the compound of Formula VIII is ethyl (Rₐ)-4-chloro-6-(2-cyanoethyl)-7-(2,3-dichlorophenyl)-8-fluoro-2-methylquinoline-3-carboxylate (Compound 8a). In some embodiments, the compound of Formula VIII is ethyl (Sₐ)-4-chloro-6-(2-cyanoethyl)-7-(2,3-dichlorophenyl)-8-fluoro-2-methylquinoline-3-carboxylate.

In some embodiments of the processes herein, the compound of Formula VIII is ethyl 6-(2-cyanoethyl)-7-(2,3-dichlorophenyl)-8-fluoro-4-hydroxy-2-methylquinoline-3-carboxylate (Compound 8b*). In still other embodiments, the compound of Formula VIII is ethyl (Rₐ)-6-(2-cyanoethyl)-7-(2,3-dichlorophenyl)-8-fluoro-4-hydroxy-2-methylquinoline-3-carboxylate (Compound 8b). In some embodiments, the compound of Formula VIII is ethyl (Sₐ)-6-(2-cyanoethyl)-7-(2,3-dichlorophenyl)-8-fluoro-4-hydroxy-2-methylquinoline-3-carboxylate.

In some embodiments of the processes herein, the compound of Formula IX is tert-butyl 5-amino-2-azabicyclo[2.1.1]hexane-2-carboxylate. In some embodiments of the processes herein, the compound of Formula IX is tert-butyl (1R,4R,5S)-5-amino-2-azabicyclo[2.1.1]hexane-2-carboxylate (Compound 9).

In some embodiments of the processes herein, the compound of Formula X is ethyl 4-chloro-6-(2-cyanovinyl)-7-(2,3-dichlorophenyl)-8-fluoro-2-methylquinoline-3-carboxylate (Compound 10*). In some embodiments of the processes herein, the compound of Formula X is ethyl (Rₐ)-4-chloro-6-(2-cyanoethyl)-7-(2,3-dichlorophenyl)-8-fluoro-2-methylquinoline-3-carboxylate (Compound 10). In some embodiments of the processes herein, the compound of Formula X is ethyl (Sₐ)-4-chloro-6-(2-cyanoethyl)-7-(2,3-dichlorophenyl)-8-fluoro-2-methylquinoline-3-carboxylate.

In some embodiments of the processes herein, the compound of Formula X-A is ethyl 4-chloro-6-(2-cyanovinyl)-7-(2,3-dichlorophenyl)-8-fluoro-2-methylquinoline-3-carboxylate. In some embodiments of the processes herein, the compound of Formula X-A is ethyl (Rₐ)-4-chloro-6-(2-cyanoethyl)-7-(2,3-dichlorophenyl)-8-fluoro-2-methylquinoline-3-carboxylate. In some embodiments of the processes herein, the compound of Formula X-A is ethyl (Sₐ)-4-chloro-6-(2-cyanoethyl)-7-(2,3-dichlorophenyl)-8-fluoro-2-methylquinoline-3-carboxylate.

In some embodiments of the processes herein, the compound of Formula XI is ethyl 6-(2-cyanovinyl)-7-(2,3-dichlorophenyl)-8-fluoro-4-hydroxy-2-methylquinoline-3-carboxylate.

In some embodiments of the processes herein, the compound of Formula XI is ethyl (Rₐ)-6-(2-cyanovinyl)-7-(2,3-dichlorophenyl)-8-fluoro-4-hydroxy-2-methylquinoline-3-carboxylate. In some embodiments of the processes herein, the compound of Formula XI is ethyl (Sₐ)-6-(2-cyanovinyl)-7-(2,3-dichlorophenyl)-8-fluoro-4-hydroxy-2-methylquinoline-3-carboxylate.

In some embodiments of the processes herein, the compound of Formula XII is ethyl 6-bromo-7-(2,3-dichlorophenyl)-8-fluoro-4-hydroxy-2-methylquinoline-3-carboxylate. In some embodiments of the processes herein, the compound of Formula XII is ethyl (Rₐ)-6-bromo-7-(2,3-dichlorophenyl)-8-fluoro-4-hydroxy-2-methylquinoline-3-carboxylate. In some embodiments of the processes herein, the compound of Formula XII is ethyl (Sₐ)-6-bromo-7-(2,3-dichlorophenyl)-8-fluoro-4-hydroxy-2-methylquinoline-3-carboxylate.

In some embodiments of the processes herein, the compound of Formula XIII is 6-bromo-7-(2,3-dichlorophenyl)-8-fluoro-2H-benzo[d][1,3]oxazine-2,4(1H)-dione. In some embodiments of the processes herein, the compound of Formula XIII is (Rₐ)-6-bromo-7-(2,3-dichlorophenyl)-8-fluoro-2H-benzo[d][1,3]oxazine-2,4(1H)-dione. In some embodiments of the processes herein, the compound of Formula XIII is (Sₐ)-6-bromo-7-(2,3-dichlorophenyl)-8-fluoro-2H-benzo[d][1,3]oxazine-2,4(1H)-dione.

In some embodiments of the processes herein, the compound of Formula XIV is ethyl 3-oxobutanoate.

In some embodiments of the processes herein, the compound of Formula XV is 3-amino-6-bromo-2′,3′-dichloro-2-fluoro-[1,1′-biphenyl]-4-carboxylic acid. In some embodiments of the processes herein, the compound of Formula XV is (Rₐ)-3-amino-6-bromo-2′,3′-dichloro-2-fluoro-[1,1′-biphenyl]-4-carboxylic acid. In some embodiments of the processes herein, the compound of Formula XV is (Sₐ)-3-amino-6-bromo-2′,3′-dichloro-2-fluoro-[1,1′-biphenyl]-4-carboxylic acid.

In some embodiments of the processes herein, the compound of Formula XVI is methyl 3-amino-6-bromo-2′,3′-dichloro-2-fluoro-[1,1′-biphenyl]-4-carboxylate. In some embodiments of the processes herein, the compound of Formula XVI is methyl (Rₐ)-3-amino-6-bromo-2′,3′-dichloro-2-fluoro-[1,1′-biphenyl]-4-carboxylate. In some embodiments of the processes herein, the compound of Formula XVI is methyl (Sₐ)-3-amino-6-bromo-2′,3′-dichloro-2-fluoro-[1,1-biphenyl]-4-carboxylate.

In some embodiments of the processes herein, the compound of Formula XVII is methyl 3-amino-2′,3′-dichloro-2-fluoro-[1,1′-biphenyl]-4-carboxylate.

In some embodiments of the processes herein, the compound of Formula XVIII is methyl 2-amino-4-bromo-3-fluorobenzoate.

In some embodiments there is provided a process of using a compound of Formula I, II, III, IV, V, VI, VII, Vill, IX, X, X, XII, XIII, XIV, XV, XVI, or XVII, or any of the embodiments thereof, in the manufacture of Compound 1*, Compound 1, or Compound 1-hemifumarate.

The process can include employing the compound as a feedstock for the manufacture. The process can include converting the compound to manufacture Compound 1*, Compound 1, or Compound 1-hemifumarate.

In some embodiments there is provided a process of using Compound 1 in the manufacture of Compound 1-hemifumarate. In some embodiments there is provided a process of using Compound 1 in the manufacture of Compound 1-hemifumarate hemimethanol. The process can include employing Compound 1 as a feedstock for the manufacture. The process can include converting Compound 1 to manufacture Compound 1-hemifumarate. The process can include converting Compound 1 to manufacture Compound 1-hemifumarate hemimethanol.

In some embodiments there is provided a process of Compound 2 in the manufacture of Compound 1 or Compound 1-hemifumarate. In some embodiments there is provided a process of Compound 2 in the manufacture of Compound 1-hemifumarate hemimethanol. The process can include employing Compound 2 as a feedstock for the manufacture. The process can include converting Compound 2 to manufacture Compound 1 or Compound 1-hemifumarate. The process can include converting Compound 2 to manufacture Compound 1-hemifumarate hemimethanol.

In some embodiments there is provided a process of using Compound 3 in the manufacture of Compound 1, Compound 1-hemifumarate, or Compound 2. In some embodiments there is provided a process of using Compound 3 in the manufacture of Compound 1 hemifumarate hemimethanol. The process can include employing Compound 3 as a feedstock for the manufacture. The process can include converting Compound 3 to manufacture Compound 1, Compound 1-hemifumarate, or Compound 2. The process can include converting Compound 3 to manufacture Compound 1 hemifumarate hemimethanol.

In some embodiments there is provided a process of using Compound 4 in the manufacture of Compound 1, Compound 1-hemifumarate, Compound 2, or Compound 3. In some embodiments there is provided a process of using Compound 4 in the manufacture of Compound 1 hemifumarate hemimethanol. The process can include employing Compound 4 as a feedstock for the manufacture. The process can include converting Compound 4 to manufacture Compound 1, Compound 1-hemifumarate, Compound 2, or Compound 3. The process can include converting Compound 4 to manufacture Compound 1 hemifumarate hemimethanol.

In some embodiments there is provided a process of using Compound 5 in the manufacture of Compound 1, Compound 1-hemifumarate, Compound 2, or Compound 3. In some embodiments there is provided a process of using Compound 5 in the manufacture of Compound 1 hemifumarate hemimethanol. The process can include employing Compound 5 as a feedstock for the manufacture. The process can include converting Compound 5 to manufacture Compound 1, Compound 1-hemifumarate, Compound 2 or Compound 3. The process can include converting Compound 5 to manufacture Compound 1 hemifumarate hemimethanol.

In some embodiments there is provided a process of using Compound 6 in the manufacture of Compound 1, Compound 1-hemifumarate, Compound 2, Compound 3 or Compound 5. In some embodiments there is provided a process of using Compound 6 in the manufacture of Compound 1 hemifumarate hemimethanol. The process can include employing Compound 6 as a feedstock for the manufacture. The process can include converting Compound 6 to manufacture Compound 1, Compound 1-hemifumarate, Compound 2, Compound 3 or Compound 5. The process can include converting Compound 6 to manufacture Compound 1 hemifumarate hemimethanol.

In some embodiments there is provided a process of using Compound 7 in the manufacture of Compound 1, Compound 1-hemifumarate, Compound 2, Compound 3, Compound 5 or Compound 6. In some embodiments there is provided a process of using Compound 7 in the manufacture of Compound 1 hemifumarate hemimethanol. The process can include employing Compound 7 as a feedstock for the manufacture. The process can include converting Compound 7 to manufacture Compound 1, Compound 1-hemifumarate, Compound 2, Compound 3, Compound 5 or Compound 6. The process can include converting Compound 7 to manufacture Compound 1 hemifumarate hemimethanol.

In some embodiments there is provided a process of using Compound 8a in the manufacture of Compound 1, Compound 1-hemifumarate, Compound 2, Compound 3, Compound 5, Compound 6 or Compound 7. In some embodiments there is provided a process of using Compound 8a in the manufacture of Compound 1 hemifumarate hemimethanol. The process can include employing Compound 8a as a feedstock for the manufacture. The process can include converting Compound 8a to manufacture Compound 1, Compound 1-hemifumarate, Compound 2, Compound 3, Compound 5, Compound 6 or Compound 7. The process can include converting Compound 8a to manufacture Compound 1 hemifumarate hemimethanol.

In some embodiments there is provided a process of using Compound 8b in the manufacture of Compound 1, Compound 1-hemifumarate, Compound 2, Compound 3, Compound 5, Compound 6 or Compound 7. In some embodiments there is provided a process of using Compound 8b in the manufacture of Compound 1 hemifumarate hemimethanol. The process can include employing Compound 8b as a feedstock for the manufacture. The process can include converting Compound 8b to manufacture Compound 1, Compound 1-hemifumarate, Compound 2, Compound 3, Compound 5, Compound 6 or Compound 7. The process can include converting Compound 8b to manufacture Compound 1 hemifumarate hemimethanol.

In some embodiments there is provided a process of using Compound 9 in the manufacture of Compound 1, Compound 1-hemifumarate, Compound 2, Compound 3, Compound 5, Compound 6 or Compound 7. In some embodiments there is provided a process of using Compound 9 in the manufacture of Compound 1 hemifumarate hemimethanol. The process can include employing Compound 9 as a feedstock for the manufacture. The process can include converting Compound 9 to manufacture Compound 1, Compound 1-hemifumarate, Compound 2, Compound 3, Compound 5, Compound 6 or Compound 7. The process can include converting Compound 9 to manufacture Compound 1 hemifumarate hemimethanol.

In some embodiments there is provided a process of using Compound 10 in the manufacture of Compound 1, Compound 1-hemifumarate, Compound 2, Compound 3, Compound 5, Compound 6, Compound 7, Compound 8a or Compound 8b. In some embodiments there is provided a process of using Compound 10 in the manufacture of Compound 1 hemifumarate hemimethanol. The process can include employing Compound 10 as a feedstock for the manufacture. The process can include converting Compound 10 to manufacture Compound 1, Compound 1-hemifumarate, Compound 2, Compound 3, Compound 5, Compound 6, Compound 7, Compound 8a or Compound 8b. The process can include converting Compound 10 to manufacture Compound 1 hemifumarate hemimethanol.

In some embodiments, an atropisomer of the compound of Formula II, III, IV, V, VI, VII, VIII, IX or X is racemized to form a mixture of atropisomers of the corresponding compound. In other embodiments, the mixture of atropisomers of the compound of Formula II, III, IV, V, VI, VII, VIII, IX or X is separated into isolated stereoisomers of the corresponding compound. In other embodiments, one stereoisomer of the compound of Formula II, III, IV, V, VI, VII, VIII, IX or X is racemized to form a second mixture of stereoisomers of the corresponding compound. In other embodiments, the second mixture of stereoisomers of the compound of Formula II, III, IV, V, VI, VII, VIII, IX or X is separated into isolated stereoisomers of the corresponding compound. By performing iterative racemization and separation (i.e., one or more racemization and separation steps following an initial separation of a racemic mixture of atropisomers) an increased yield of one or other of the atropisomers (i.e., the Rₐ or Sₐ atropsomer) of the compound of Formula II, III, IV, V, VI, VII, VIII, IX or X can be obtained compared to a single chiral separation to isolate a single atropisomer from a racemic mixture.

The present disclosure also provides the full synthesis of a compound wherein multiple steps provided herein are combined in sequence. For example, provided herein is a process comprising forming a compound of Formula I from the starting material of a compound of Formula V using the disclosed synthesis procedures. In some embodiments, the racemization can be carried out at a temperature in the range from about 0° C. to about 120° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., or about 120° C. In some embodiments, the racemization can be carried out at a temperature from about 50° C. to about 90° C., or from about 60° C. to about 80° C., such as at about 70° C. In some embodiments, the racemization can be carried out at reflux.

In other embodiments, the racemization can be carried out in the presence of a suitable solvent, or mixtures thereof based on the solubility of the compounds described herein. Suitable solvents include, but are not limited to, DCM, THF, 1,4-dioxane, MeCN, toluene, tert-butanol, pyridine, N,N-diethylaniline, xylene, and DMSO, or combinations thereof.

In other embodiments, the process comprises chiral separation. In yet other embodiments, the process comprises separating atropisomers of a compound of Formula I, II, III, IV, V, VI, VII, VIII, IX, or X. In some embodiments, the separating can be performed using chromatography such as by HPLC or supercritical fluid chromatography. In some embodiments, the chromatography is performed using a chiral stationary phase. In some embodiments, the chiral stationary phase is a Pirkle type (Brush type) chiral stationary phase. In some embodiments, the chiral stationary phase is a protein-based chiral stationary phase. In some embodiments, the chiral stationary phase is a cyclodextrin-based chiral stationary phase. In some embodiments, the chiral stationary phase is a polymer-based carbohydrate chiral stationary phase (polysaccharide-based). In some embodiments, the chiral stationary phase is a macrocyclic antibiotic chiral stationary phase. In some embodiments, the chiral stationary phase is a chiral crown ether chiral stationary phase. In some embodiments, the chiral stationary phase is an imprinted polymer chiral stationary phase.

### III. Intermediate Compounds

In an aspect, provided herein is a compound of Formula II:

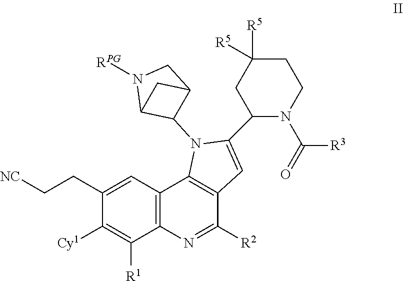

wherein
   R^{PG} is a nitrogen protecting group;
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen;
   R² is C₁₋₃ alkyl optionally substituted with OH;
   R³ is OR^{3A};
   R^{3A} is selected from C₁₋₃ alkyl, C₂₋₃ alkenyl, and C₂₋₃ alkynyl;
   each R⁵ is independently selected from H, D, halo, C₁₋₃ alkyl, OR^{a5}, C₁₋₃ haloalkyl, C₂₋₃ alkenyl, and C₂₋₃ alkynyl; and
   each R^{a5} is independently selected from H, C₁₋₃ alkyl, and C₁₋₃ haloalkyl.

In some embodiments, R¹ is fluoro.

In other embodiments, Cy¹ is 2,3-dichlorophenyl.

In yet other embodiments, R^{3A} is C₁₋₃ alkyl. In still other embodiments, R^{3A} is methyl.

In some embodiments, one R⁵ is H. In some embodiments, one R⁵ is OR^{a5}. In some embodiments, R^{a5} is C₁₋₃ haloalkyl. In some embodiments, R^{a5} is CHF₂.

In other embodiments, R^{PG} is benzyloxycarbonyl (Cbz), 2,2,2-trichloroethoxycarbonyl (Troc), 2-(trimethylsilyl)ethoxycarbonyl (Teoc), tert-butoxycarbonyl (Boc), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, benzyloxymethyl (BOM), or 2-tetrahydropyranyl (THP).

In other embodiments, R^{PG} is tert-butyloxycarbonyl.

In yet other embodiments, the compound of Formula II is tert-butyl 5-(8-(2-cyanoethyl)-7-(2,3-dichlorophenyl)-2-(5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate. In still other embodiments, the compound of Formula II is tert-butyl (1R,4R,5S)-5-(8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate. In some embodiments, wherein the compound of Formula II is tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate. In other embodiments, the compound of Formula II is tert-butyl (1R,4R,5S)-5-((Sₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate.

In another aspect, provided herein is a compound of Formula III:

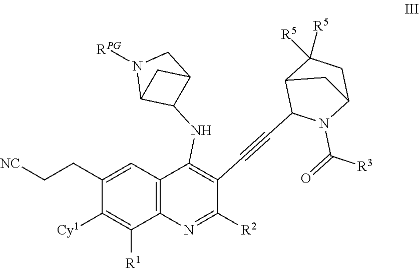

wherein
   R^{PG} is a nitrogen protecting group;
   Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
   R¹ is halogen;
   R² is C₁₋₃ alkyl optionally substituted with OH;
   R³ is OR^{3A};
   R^{3A} is selected from C₁₋₃ alkyl, C₂₋₃ alkenyl, and C₂₋₃ alkynyl;
   each R⁵ is independently selected from H, D, halo, C₁₋₃ alkyl, OR^{a}s, C₁₋₃ haloalkyl, C₂₋₃ alkenyl, and C₂₋₃ alkynyl; and
   each R^{a5} is independently selected from H, C₁₋₃ alkyl, and C₁₋₃ haloalkyl.

In some embodiments, R¹ is fluoro.

In other embodiments, Cy¹ is 2,3-dichlorophenyl.

In yet other embodiments, R^{3A} is C₁₋₃ alkyl. In other embodiments, R^{3A} is methyl.

In other embodiments, one R⁵ is H. In other embodiments, one R⁵ is OR^{a5}.

In other embodiments, R^{a5} is C₁₋₃ haloalkyl. In other embodiments, R^{a5} is CHF₂.

In other embodiments, R^{PG} is tert-butyloxycarbonyl.

In other embodiments, R^{PG} is benzyloxycarbonyl (Cbz), 2,2,2-trichloroethoxycarbonyl (Troc), 2-(trimethylsilyl)ethoxycarbonyl (Teoc), tert-butoxycarbonyl (Boc), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, benzyloxymethyl (BOM), or 2-tetrahydropyranyl (THP).

In yet other embodiments, the compound of Formula III is tert-butyl 5-((6-(2-cyanoethyl)-7-(2,3-dichlorophenyl)-3-((5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate. In still other embodiments, the compound of Formula III is tert-butyl (1R,4R,5S)-5-((6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate. In some embodiments, the compound of Formula III is tert-butyl (1R,4R,5S)-5-(((Rₐ)-6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate. In other embodiments, the compound of Formula III is tert-butyl (1R,4R,5S)-5-(((Sₐ)-6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate.

### IV. Salts and Crystalline Forms

### 1. Characterization of Crystalline Forms

In certain embodiments, the crystalline forms described herein are identifiable on the basis of characteristic peaks in an X-ray powder diffraction analysis. X-ray powder diffraction (XRPD) is a scientific technique using X-ray, neutron, or electron diffraction on powder, microcrystalline, or other solid materials for structural characterization of solid materials. A description of the methods used to obtain certain XRPD diffractograms in connection with the crystalline forms provided herein can be found in the Examples below. In an embodiment, the X-ray powder diffraction data provided herein is obtained by a method utilizing Cu Kα radiation.

Thus, in an aspect, provided herein is a compound that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof, wherein the compound is crystalline.

In an embodiment, the compound or pharmaceutically acceptable salt thereof is a pharmaceutically acceptable salt thereof. In another embodiment, the pharmaceutically acceptable salt is selected from fumarate, hemifumarate, hydrochloride, di-hydrochloride, L-tartrate, and phosphate. In another embodiment, the pharmaceutically acceptable salt is hemimethanol. In another embodiment, the pharmaceutically acceptable salt is hemifumarate hemimethanol.

In another embodiment, the compound or pharmaceutically acceptable salt thereof is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 1).

In another embodiment, the compound or pharmaceutically acceptable salt thereof is methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate.

In an embodiment, the compound or pharmaceutically acceptable salt thereof is a solvate. In another embodiment, the compound or pharmaceutically acceptable salt thereof is a hydrate.

### Compound 1 Hemifumarate

In an embodiment, the compound or pharmaceutically acceptable salt thereof is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate.

In another embodiment, the compound or pharmaceutically acceptable salt thereof is characterized by an XRPD diffractogram having at least one (or two) of the following peaks expressed in degrees-2-theta at angles (±0.2 degrees) of 6.3, 6.7, 13.0, 17.1, 18.1, 19.4, 20.3, 23.8, 24.9, and 25.4.

In yet another embodiment, the compound or pharmaceutically acceptable salt thereof is characterized by an XRPD diffractogram having at least three (or at least four, five, six, seven, eight, nine, or all) of the following peaks expressed in degrees-2-theta at angles (±0.2 degrees) of 6.3, 6.7, 13.0, 17.1, 18.1, 19.4, 20.3, 23.8, 24.9, and 25.4.

In still another embodiment, the compound or pharmaceutically acceptable salt thereof is characterized by an XRPD diffractogram having peaks expressed in degrees-2-theta at angles (±0.2 degrees) of 6.3, 6.7, and 17.1.

In still another embodiment, the compound or pharmaceutically acceptable salt thereof is characterized by an XRPD diffractogram having peaks expressed in degrees-2-theta at angles (±0.2 degrees) of 6.3, 6.7, 13.0, and 17.1.

In an embodiment, the compound or pharmaceutically acceptable salt thereof is characterized by an XRPD diffractogram having at least six of the following peaks expressed in degrees-2-theta at angles (±0.2 degrees) of 6.3, 6.7, 13.0, 17.1, 18.1, 19.4, 20.3, 23.8, 24.9, and 25.4. In another embodiment, the compound or pharmaceutically acceptable salt thereof is characterized by an XRPD diffractogram having peaks expressed in degrees-2-theta at angles (±0.2 degrees) of 6.3, 6.7, 13.0, 17.1, 23.8, and 24.9.

In yet another embodiment, the compound or pharmaceutically acceptable salt thereof is characterized by an XRPD diffractogram having peaks expressed in degrees-2-theta at angles (±0.2 degrees) of 6.3, 6.7, 13.0, 17.1, 18.1, 19.4, 20.3, 23.8, 24.9, and 25.4.

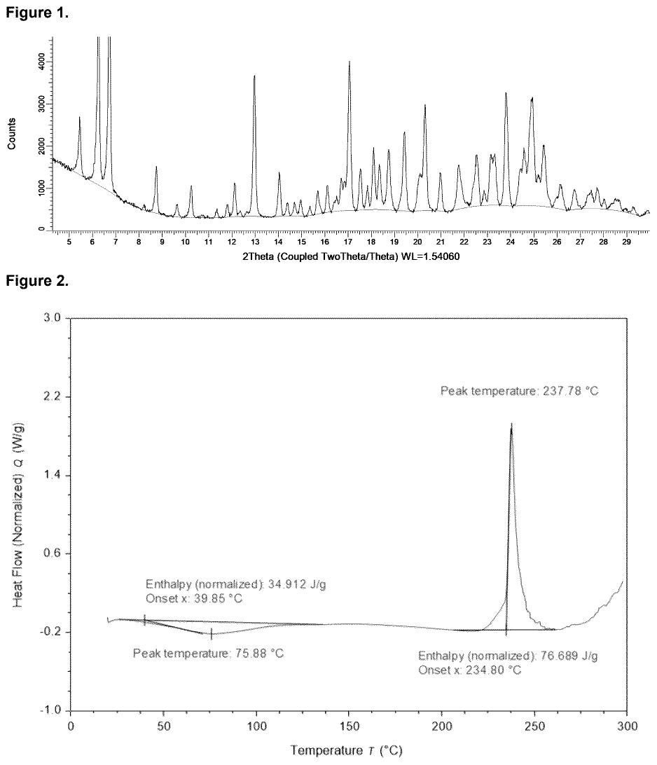

In another embodiment, the compound or pharmaceutically acceptable salt thereof is characterized by an XRPD diffractogram substantially as depicted in FIG. 1.

In another embodiment, the compound or pharmaceutically acceptable salt thereof is characterized by an XRPD diffractogram having peaks expressed in degrees-2-theta at angles (±0.2 degrees) selected from the list in Table 1.

TABLE 1

| 2-Theta (°) | H % |
|---|---|
| 5.5 | 24.7 |
| 6.3 | 82.8 |
| 6.7 | 100 |
| 8.2 | 2.1 |
| 8.7 | 20.1 |
| 9.6 | 5.2 |
| 10.2 | 13.5 |
| 10.7 | 1.2 |
| 11.4 | 3.9 |
| 11.8 | 5.5 |
| 12.1 | 14.7 |
| 12.4 | 2.6 |
| 12.7 | 2.1 |
| 13.0 | 62.4 |
| 14.0 | 19.3 |
| 14.4 | 5.5 |
| 14.7 | 6.1 |
| 15.0 | 7.0 |
| 15.4 | 4.1 |
| 15.7 | 10.4 |
| 16.1 | 12.0 |
| 16.5 | 6.6 |
| 16.7 | 14.5 |
| 17.1 | 65.8 |
| 17.5 | 18.0 |
| 17.8 | 10.5 |
| 18.1 | 27.0 |
| 18.4 | 19.6 |
| 18.8 | 26.3 |
| 19.4 | 34.9 |
| 20.1 | 16.2 |
| 20.3 | 47.0 |
| 21.0 | 16.6 |
| 21.8 | 19.6 |
| 22.5 | 22.7 |
| 22.9 | 6.7 |
| 23.2 | 20.9 |
| 23.8 | 50.1 |
| 24.4 | 16.2 |
| 24.6 | 25.4 |
| 24.9 | 47.4 |
| 25.4 | 26.6 |
| 26.0 | 4.5 |
| 26.2 | 10.7 |
| 26.7 | 8.6 |
| 27.5 | 7.5 |
| 27.7 | 9.2 |
| 28.0 | 3.9 |
| 28.6 | 5.1 |
| 28.9 | 1.4 |
| 29.3 | 3.0 |

In another embodiment, the compound or pharmaceutically acceptable salt thereof has a DSC thermogram characterized by an exotherm substantially as depicted in FIG. 2.

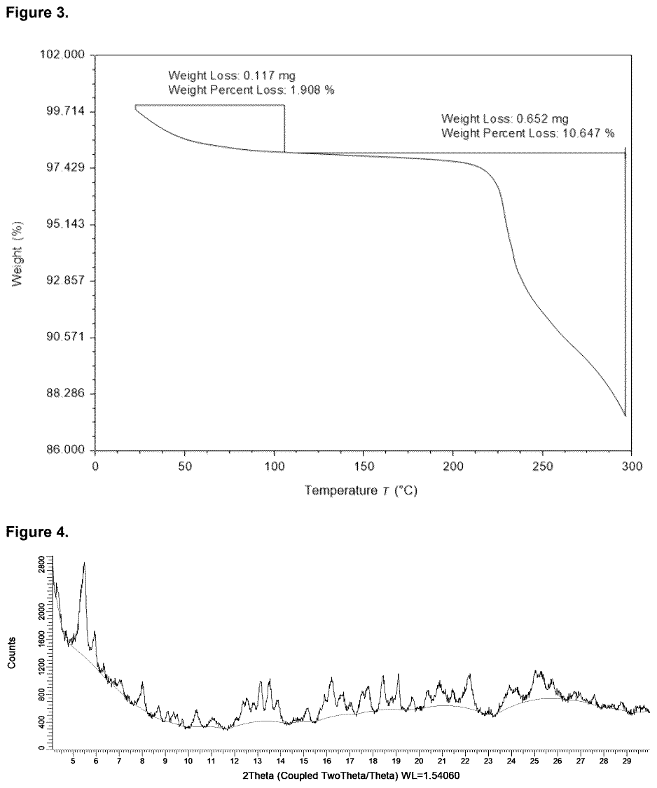

In another embodiment, the compound or pharmaceutically acceptable salt thereof has a DSC thermogram characterized by an exotherm having a peak at about 238° C. In another embodiment, the compound or pharmaceutically acceptable salt thereof has a DSC thermogram characterized by an exotherm having an onset at about 235° C. In another embodiment, the compound or pharmaceutically acceptable salt thereof has a DSC thermogram characterized by a dehydration event below about 150° C. In yet another embodiment, the compound or pharmaceutically acceptable salt thereof has a TGA thermogram substantially as depicted in FIG. 3.

### Compound 1 Di-Hydrochloride

In an embodiment, the compound or pharmaceutically acceptable salt thereof is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate di-hydrochloride.

In another embodiment, the compound or pharmaceutically acceptable salt thereof is characterized by an XRPD diffractogram having at least one (or two) of the following peaks expressed in degrees-2-theta at angles (±0.2 degrees) of 5.5, 5.9, 8.0, 13.1, 13.5, 16.2, 18.4, 19.1, 22.2, and 25.3.

In yet another embodiment, the compound or pharmaceutically acceptable salt thereof is characterized by an XRPD diffractogram having at least three (or at least four, five, six, seven, eight, nine, or all) of the following peaks expressed in degrees-2-theta at angles (±0.2 degrees) of 5.5, 5.9, 8.0, 13.1, 13.5, 16.2, 18.4, 19.1, 22.2, and 25.3.

In still another embodiment, the compound or pharmaceutically acceptable salt thereof is characterized by an XRPD diffractogram having peaks expressed in degrees-2-theta at angles (±0.2 degrees) of 5.5, 13.1, and 16.2.

In still another embodiment, the compound or pharmaceutically acceptable salt thereof is characterized by an XRPD diffractogram having peaks expressed in degrees-2-theta at angles (±0.2 degrees) of 5.5, 13.1, 13.5, and 16.2.

In an embodiment, the compound or pharmaceutically acceptable salt thereof is characterized by an XRPD diffractogram having at least six of the following peaks expressed in degrees-2-theta at angles (±0.2 degrees) of 5.5, 5.9, 8.0, 13.1, 13.5, 16.2, 18.4, 19.1, 22.2, and 25.3. In another embodiment, the compound or pharmaceutically acceptable salt thereof is characterized by an XRPD diffractogram having peaks expressed in degrees-2-theta at angles (±0.2 degrees) of 5.5, 5.9, 13.1, 13.5, 16.2, and 19.1.

In yet another embodiment, the compound or pharmaceutically acceptable salt thereof is characterized by an XRPD diffractogram having peaks expressed in degrees-2-theta at angles (±0.2 degrees) of 5.5, 5.9, 8.0, 13.1, 13.5, 16.2, 18.4, 19.1, 22.2, and 25.3.

In another embodiment, the compound or pharmaceutically acceptable salt thereof is characterized by an XRPD diffractogram substantially as depicted in FIG. 4.

In another embodiment, the compound or pharmaceutically acceptable salt thereof is characterized by an XRPD diffractogram having peaks expressed in degrees-2-theta at angles (±0.2 degrees) selected from the list in Table 2.

TABLE 2

| 2-Theta (°) | H % |
|---|---|
| 4.3 | 15.6 |
| 5.5 | 100 |
| 5.9 | 42.5 |
| 6.3 | 12.4 |
| 7.0 | 12.8 |
| 8.0 | 33.2 |
| 8.7 | 13.3 |
| 9.1 | 12.3 |
| 9.4 | 13.4 |
| 9.5 | 10.6 |
| 9.8 | 7.7 |
| 10.4 | 19.8 |
| 11.0 | 11.5 |
| 12.1 | 8.6 |
| 12.4 | 24.4 |
| 12.5 | 29.1 |
| 12.8 | 22.1 |
| 13.1 | 47.4 |
| 13.5 | 46.5 |
| 13.9 | 24.8 |
| 15.1 | 15.6 |
| 15.9 | 28.7 |
| 16.2 | 46.7 |
| 16.6 | 23.3 |
| 17.0 | 14.7 |
| 17.6 | 23.3 |
| 17.8 | 29.8 |
| 18.4 | 40.4 |
| 19.1 | 42.5 |
| 19.7 | 14.1 |
| 20.4 | 19.9 |
| 20.9 | 26.0 |
| 21.4 | 17.7 |
| 22.2 | 39.2 |
| 23.0 | 6.5 |
| 23.9 | 22.8 |
| 24.2 | 19.8 |
| 25.0 | 32.4 |
| 25.3 | 32.7 |
| 25.8 | 23.4 |
| 26.7 | 10.3 |
| 27.6 | 12.6 |
| 28.7 | 10.0 |

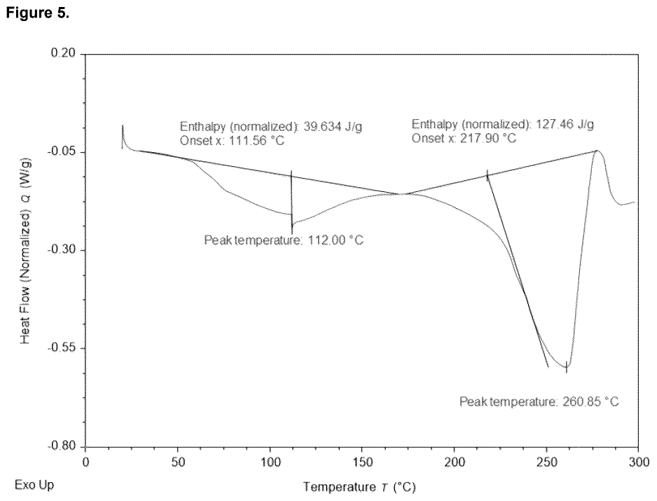

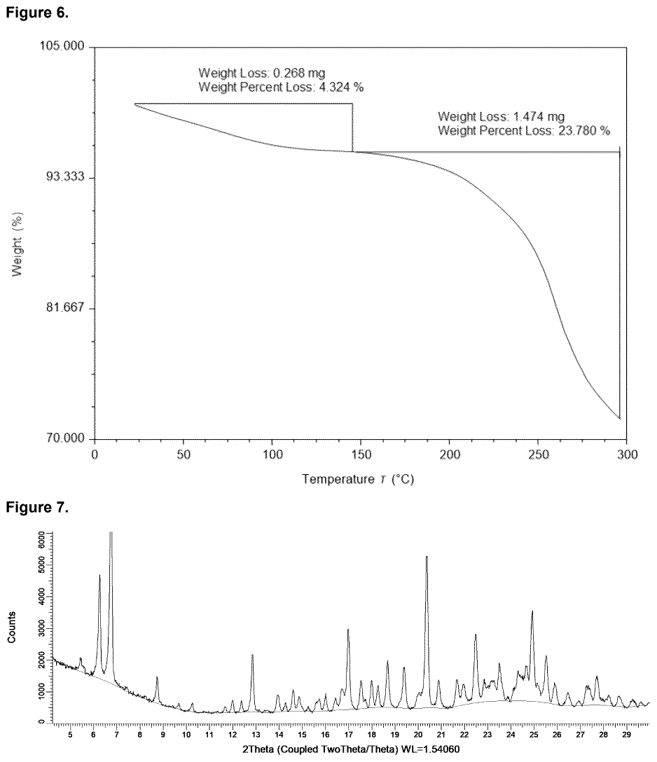

In another embodiment, the compound or pharmaceutically acceptable salt thereof has a DSC thermogram characterized by an endotherm substantially as depicted in FIG. 5. In another embodiment, the compound or pharmaceutically acceptable salt thereof has a DSC thermogram characterized by an endotherm having a peak at about 261° C. In another embodiment, the compound or pharmaceutically acceptable salt thereof has a DSC thermogram characterized by an endotherm having an onset at about 218° C. In another embodiment, the compound or pharmaceutically acceptable salt thereof has a DSC thermogram characterized by a dehydration event below about 150° C. In yet another embodiment, the compound or pharmaceutically acceptable salt thereof has a TGA thermogram substantially as depicted in FIG. 6.

In an aspect, provided herein is a process for preparing methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate di-hydrochloride comprising reacting methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate with a stoichiometric excess of hydrochloric acid to produce methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate di-hydrochloride.

### Compound 1 Hydrochloride

In an embodiment, the compound or pharmaceutically acceptable salt thereof is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hydrochloride.

In another embodiment, the compound or pharmaceutically acceptable salt thereof is characterized by an XRPD diffractogram having at least one (or two) of the following peaks expressed in degrees-2-theta at angles (±0.2 degrees) of 6.3, 6.8, 12.9, 17.0, 18.7, 19.4, 20.4, 22.5, 24.9, and 25.5.

In yet another embodiment, the compound or pharmaceutically acceptable salt thereof is characterized by an XRPD diffractogram having at least three (or at least four, five, six, seven, eight, nine, or all) of the following peaks expressed in degrees-2-theta at angles (±0.2 degrees) of 6.3, 6.8, 12.9, 17.0, 18.7, 19.4, 20.4, 22.5, 24.9, and 25.5.

In still another embodiment, the compound or pharmaceutically acceptable salt thereof is characterized by an XRPD diffractogram having peaks expressed in degrees-2-theta at angles (±0.2 degrees) of 6.3, 6.8, and 20.4.

In still another embodiment, the compound or pharmaceutically acceptable salt thereof is characterized by an XRPD diffractogram having peaks expressed in degrees-2-theta at angles (±0.2 degrees) of 6.3, 6.8, 20.4, and 24.9.

In an embodiment, the compound or pharmaceutically acceptable salt thereof is characterized by an XRPD diffractogram having at least six of the following peaks expressed in degrees-2-theta at angles (±0.2 degrees) of 6.3, 6.8, 12.9, 17.0, 18.7, 19.4, 20.4, 22.5, 24.9, and 25.5. In another embodiment, the compound or pharmaceutically acceptable salt thereof is characterized by an XRPD diffractogram having peaks expressed in degrees-2-theta at angles (±0.2 degrees) of 6.3, 6.8, 17.0, 20.4, 22.5, and 24.9.

In yet another embodiment, the compound or pharmaceutically acceptable salt thereof is characterized by an XRPD diffractogram having peaks expressed in degrees-2-theta at angles (±0.2 degrees) of 6.3, 6.8, 12.9, 17.0, 18.7, 19.4, 20.4, 22.5, 24.9, and 25.5.

In another embodiment, the compound or pharmaceutically acceptable salt thereof is characterized by an XRPD diffractogram substantially as depicted in FIG. 7.

In another embodiment, the compound or pharmaceutically acceptable salt thereof is characterized by an XRPD diffractogram having peaks expressed in degrees-2-theta at angles (±0.2 degrees) selected from the list in Table 3.

TABLE 3

| 2-Theta (°) | H % |
|---|---|
| 5.4 | 4.9 |
| 6.3 | 39.8 |
| 6.8 | 100 |
| 7.4 | 1.1 |
| 8.7 | 10.0 |
| 9.7 | 2.2 |
| 10.3 | 3.4 |
| 11.3 | 0.7 |
| 11.7 | 2.2 |
| 12.0 | 4.7 |
| 12.4 | 4.5 |
| 12.9 | 22.2 |
| 13.5 | 0.7 |
| 13.9 | 6.5 |
| 14.3 | 3.3 |
| 14.6 | 8.3 |
| 14.9 | 5.6 |
| 15.2 | 1.8 |
| 15.7 | 4.8 |
| 16.0 | 6.6 |
| 16.4 | 4.9 |
| 16.7 | 7.9 |
| 17.0 | 30.9 |
| 17.5 | 10.8 |
| 18.0 | 10.5 |
| 18.3 | 8.2 |
| 18.7 | 17.5 |
| 19.4 | 15.5 |
| 20.1 | 6.1 |
| 20.4 | 58.3 |
| 20.9 | 10.7 |
| 21.7 | 10.2 |
| 22.0 | 7.8 |
| 22.5 | 26.5 |
| 22.9 | 7.3 |
| 23.2 | 7.8 |
| 23.5 | 14.4 |
| 23.9 | 1.9 |
| 24.2 | 5.6 |
| 24.4 | 9.8 |
| 24.7 | 13.4 |
| 24.9 | 34.7 |
| 25.2 | 6.1 |
| 25.5 | 18.0 |
| 25.9 | 8.1 |
| 26.5 | 5.2 |
| 26.9 | 1.9 |
| 27.3 | 7.5 |
| 27.7 | 10.3 |
| 28.2 | 3.6 |
| 28.7 | 3.9 |
| 29.3 | 3.0 |
| 29.6 | 1.7 |

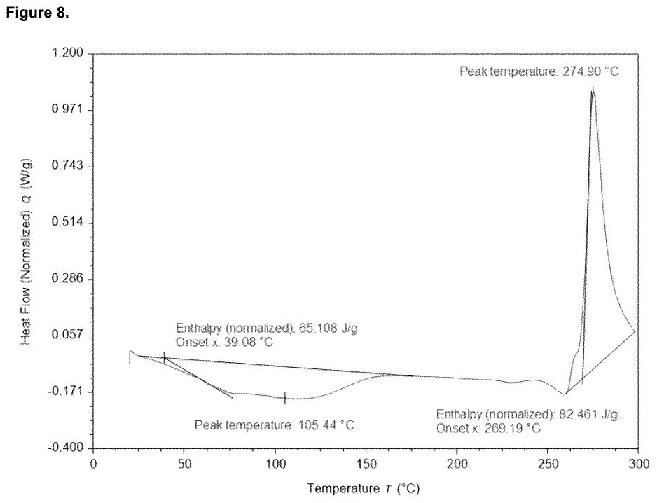

In another embodiment, the compound or pharmaceutically acceptable salt thereof has a DSC thermogram characterized by an exotherm substantially as depicted in FIG. 8.

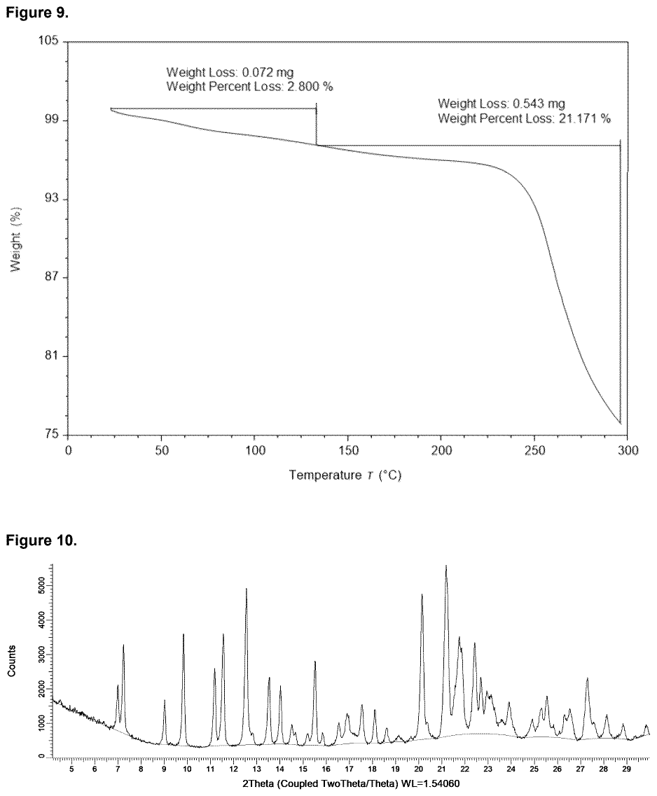

In another embodiment, the compound or pharmaceutically acceptable salt thereof has a DSC thermogram characterized by an exotherm having a peak at about 275° C. In another embodiment, the compound or pharmaceutically acceptable salt thereof has a DSC thermogram characterized by an exotherm having an onset at about 269° C. In another embodiment, the compound or pharmaceutically acceptable salt thereof has a DSC thermogram characterized by a dehydration event below about 150° C. In yet another embodiment, the compound or pharmaceutically acceptable salt thereof has a TGA thermogram substantially as depicted in FIG. 9.

In an aspect, provided herein is a process for preparing methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hydrochloride comprising reacting methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate with one stoichiometric equivalent of hydrochloric acid to produce methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hydrochloride.

### Compound 1 L-tartrate

In an embodiment, the compound or pharmaceutically acceptable salt thereof is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate L-tartrate.

In another embodiment, the compound or pharmaceutically acceptable salt thereof is characterized by an XRPD diffractogram having at least one (or two) of the following peaks expressed in degrees-2-theta at angles (±0.2 degrees) of 7.2, 9.8, 11.6, 12.7, 13.5, 15.5, 20.2, 21.2, 21.8, and 22.4.

In yet another embodiment, the compound or pharmaceutically acceptable salt thereof is characterized by an XRPD diffractogram having at least three (or at least four, five, six, seven, eight, nine, or all) of the following peaks expressed in degrees-2-theta at angles (±0.2 degrees) of 7.2, 9.8, 11.6, 12.7, 13.5, 15.5, 20.2, 21.2, 21.8, and 22.4.

In still another embodiment, the compound or pharmaceutically acceptable salt thereof is characterized by an XRPD diffractogram having peaks expressed in degrees-2-theta at angles (±0.2 degrees) of 12.7, 21.2, and 21.2.

In still another embodiment, the compound or pharmaceutically acceptable salt thereof is characterized by an XRPD diffractogram having peaks expressed in degrees-2-theta at angles (±0.2 degrees) of 9.8, 12.7, 21.2, and 21.2.

In an embodiment, the compound or pharmaceutically acceptable salt thereof is characterized by an XRPD diffractogram having at least six of the following peaks expressed in degrees-2-theta at angles (±0.2 degrees) of 7.2, 9.8, 11.6, 12.7, 13.5, 15.5, 20.2, 21.2, 21.8, and 22.4. In another embodiment, the compound or pharmaceutically acceptable salt thereof is characterized by an XRPD diffractogram having peaks expressed in degrees-2-theta at angles (±0.2 degrees) of 9.8, 11.6, 12.7, 21.2, 21.2, and 22.4.

In yet another embodiment, the compound or pharmaceutically acceptable salt thereof is characterized by an XRPD diffractogram having peaks expressed in degrees-2-theta at angles (±0.2 degrees) of 7.2, 9.8, 11.6, 12.7, 13.5, 15.5, 20.2, 21.2, 21.8, and 22.4.

In another embodiment, the compound or pharmaceutically acceptable salt thereof is characterized by an XRPD diffractogram substantially as depicted in FIG. 10.

In another embodiment, the compound or pharmaceutically acceptable salt thereof is characterized by an XRPD diffractogram having peaks expressed in degrees-2-theta at angles (±0.2 degrees) selected from the list in Table 4.

TABLE 4

| 2-Theta (°) | H % |
|---|---|
| 4.5 | 2.8 |
| 7.0 | 26.9 |
| 7.2 | 51.5 |
| 9.0 | 25.8 |
| 9.8 | 65.4 |
| 11.2 | 45.3 |
| 11.6 | 65.1 |
| 12.6 | 91.0 |
| 12.7 | 7.8 |
| 13.5 | 39.0 |
| 14.0 | 34.1 |
| 14.5 | 11.7 |
| 14.7 | 7.3 |
| 15.2 | 6.8 |
| 15.5 | 49.1 |
| 15.8 | 7.6 |
| 16.6 | 12.7 |
| 16.9 | 17.4 |
| 17.5 | 22.4 |
| 18.1 | 19.6 |
| 18.6 | 8.3 |
| 19.1 | 4.1 |
| 19.7 | 2.6 |
| 20.2 | 84.8 |
| 20.4 | 9.6 |
| 21.2 | 100 |
| 21.8 | 52.4 |
| 22.4 | 53.0 |
| 22.7 | 32.9 |
| 23.0 | 21.8 |
| 23.1 | 22.5 |
| 23.6 | 8.0 |
| 23.9 | 19.7 |
| 24.9 | 10.4 |
| 25.3 | 16.5 |
| 25.5 | 23.9 |
| 25.8 | 7.4 |
| 26.4 | 11.3 |
| 26.5 | 17.7 |
| 27.3 | 35.6 |
| 27.5 | 8.8 |
| 28.1 | 13.9 |
| 28.8 | 8.8 |
| 29.4 | 1.4 |
| 29.8 | 5.1 |

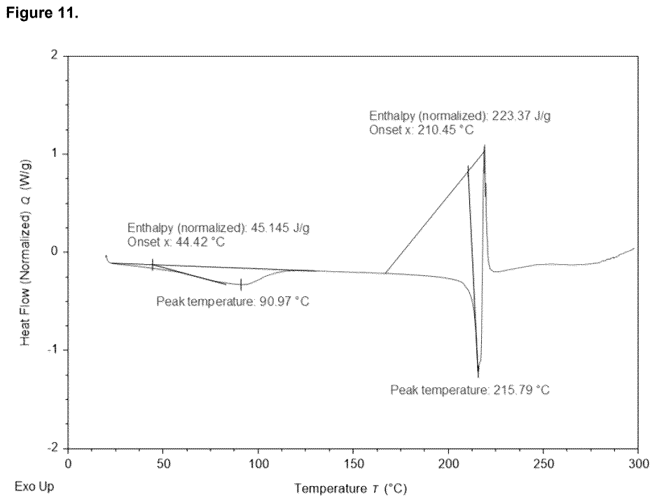

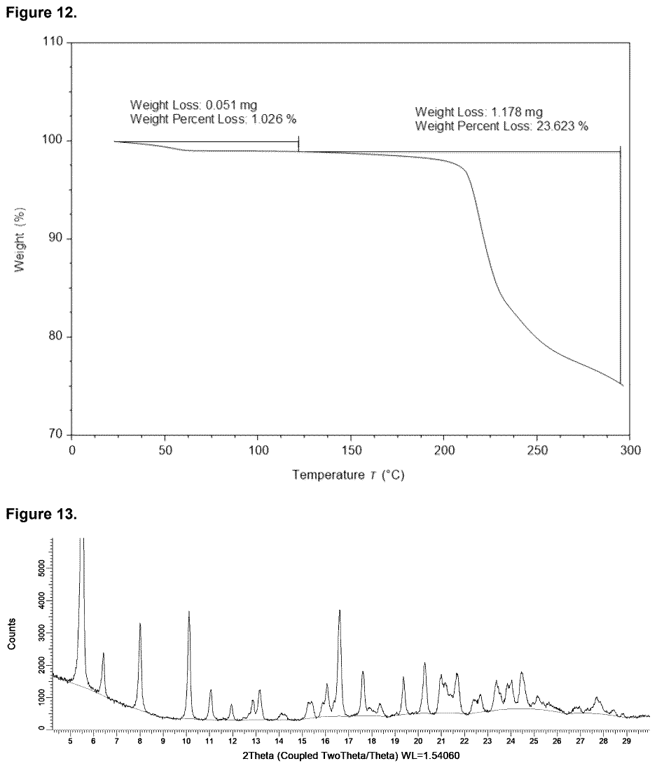

In another embodiment, the compound or pharmaceutically acceptable salt thereof has a DSC thermogram characterized by an exotherm substantially as depicted in FIG. 11. In another embodiment, the compound or pharmaceutically acceptable salt thereof has a DSC thermogram characterized by an exotherm having a peak at about 223° C. In another embodiment, the compound or pharmaceutically acceptable salt thereof has a DSC thermogram characterized by an exotherm having an onset at about 211° C. In another embodiment, the compound or pharmaceutically acceptable salt thereof has a DSC thermogram characterized by a dehydration event below about 150° C. In yet another embodiment, the compound or pharmaceutically acceptable salt thereof has a TGA thermogram substantially as depicted in FIG. 12.

In an aspect, provided herein is a process for preparing methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate L-tartrate comprising reacting methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate with L-tartaric acid to produce methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate L-tartrate.

### Compound 1 Phosphate

In an embodiment, the compound or pharmaceutically acceptable salt thereof is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate phosphate.

In another embodiment, the compound or pharmaceutically acceptable salt thereof is characterized by an XRPD diffractogram having at least one (or two) of the following peaks expressed in degrees-2-theta at angles (±0.2 degrees) of 5.5, 6.4, 8.0, 10.1, 16.6, 17.6, 19.4, 21.0, 21.7, and 23.3.

In yet another embodiment, the compound or pharmaceutically acceptable salt thereof is characterized by an XRPD diffractogram having at least three (or at least four, five, six, seven, eight, nine, or all) of the following peaks expressed in degrees-2-theta at angles (±0.2 degrees) of 5.5, 6.4, 8.0, 10.1, 16.6, 17.6, 19.4, 21.0, 21.7, and 23.3.

In still another embodiment, the compound or pharmaceutically acceptable salt thereof is characterized by an XRPD diffractogram having peaks expressed in degrees-2-theta at angles (±0.2 degrees) of 5.5, 10.1, and 16.6.

In still another embodiment, the compound or pharmaceutically acceptable salt thereof is characterized by an XRPD diffractogram having peaks expressed in degrees-2-theta at angles (±0.2 degrees) of 5.5, 8.0, 10.1, and 16.6.

In an embodiment, the compound or pharmaceutically acceptable salt thereof is characterized by an XRPD diffractogram having at least six of the following peaks expressed in degrees-2-theta at angles (±0.2 degrees) of 5.5, 6.4, 8.0, 10.1, 16.6, 17.6, 19.4, 21.0, 21.7, and 23.3. In another embodiment, the compound or pharmaceutically acceptable salt thereof is characterized by an XRPD diffractogram having peaks expressed in degrees-2-theta at angles (±0.2 degrees) of 5.5, 8.0, 10.1, 16.6, 17.6, and 20.3.

In yet another embodiment, the compound or pharmaceutically acceptable salt thereof is characterized by an XRPD diffractogram having peaks expressed in degrees-2-theta at angles (±0.2 degrees) of 5.5, 6.4, 8.0, 10.1, 16.6, 17.6, 19.4, 21.0, 21.7, and 23.3.

In another embodiment, the compound or pharmaceutically acceptable salt thereof is characterized by an XRPD diffractogram substantially as depicted in FIG. 13.

In another embodiment, the compound or pharmaceutically acceptable salt thereof is characterized by an XRPD diffractogram having peaks expressed in degrees-2-theta at angles (±0.2 degrees) selected from the list in Table 5.

TABLE 5

| 2-Theta (°) | H % |
|---|---|
| 5.5 | 100 |
| 6.4 | 9.4 |
| 8.0 | 19.4 |
| 10.1 | 24.1 |
| 11.1 | 6.7 |
| 11.9 | 3.5 |
| 12.5 | 0.6 |
| 12.9 | 4.3 |
| 13.2 | 6.5 |
| 14.2 | 1.2 |
| 15.3 | 3.2 |
| 15.9 | 3.2 |
| 16.1 | 7.3 |
| 16.6 | 23.7 |
| 17.6 | 9.9 |
| 17.9 | 1.8 |
| 18.4 | 2.6 |
| 19.4 | 8.5 |
| 20.3 | 11.4 |
| 21.0 | 8.7 |
| 21.2 | 6.8 |
| 21.7 | 8.9 |
| 22.5 | 3.0 |
| 22.7 | 3.8 |
| 23.4 | 6.5 |
| 23.9 | 5.8 |
| 24.0 | 6.4 |
| 24.5 | 8.0 |
| 25.2 | 3.0 |
| 25.6 | 1.9 |
| 26.0 | 0.7 |
| 26.8 | 1.4 |
| 26.9 | 1.3 |
| 27.3 | 1.1 |
| 27.7 | 3.7 |
| 27.8 | 2.7 |
| 28.4 | 1.3 |
| 28.8 | 0.5 |

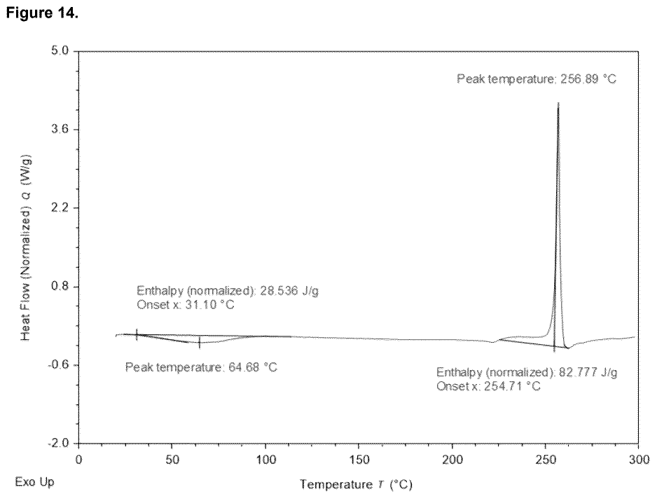

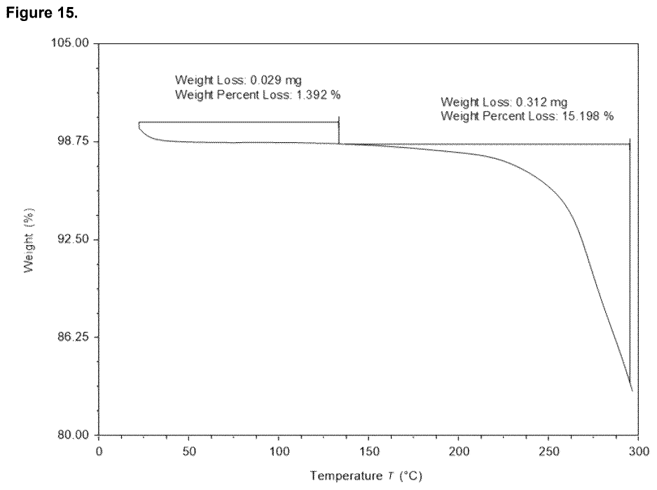

In another embodiment, the compound or pharmaceutically acceptable salt thereof has a DSC thermogram characterized by an exotherm substantially as depicted in FIG. 14. In another embodiment, the compound or pharmaceutically acceptable salt thereof has a DSC thermogram characterized by an exotherm having a peak at about 257° C. In another embodiment, the compound or pharmaceutically acceptable salt thereof has a DSC thermogram characterized by an exotherm having an onset at about 255° C. In another embodiment, the compound or pharmaceutically acceptable salt thereof has a DSC thermogram characterized by a dehydration event below about 150° C. In yet another embodiment, the compound or pharmaceutically acceptable salt thereof has a TGA thermogram substantially as depicted in FIG. 15.

In an aspect, provided herein is a process for preparing methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate phosphate comprising reacting methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate with phosphoric acid to produce methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate phosphate.

### Compound 1 Hemifumarate Hemimethanol

In an embodiment, the compound or pharmaceutically acceptable salt thereof is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate hemimethanol.

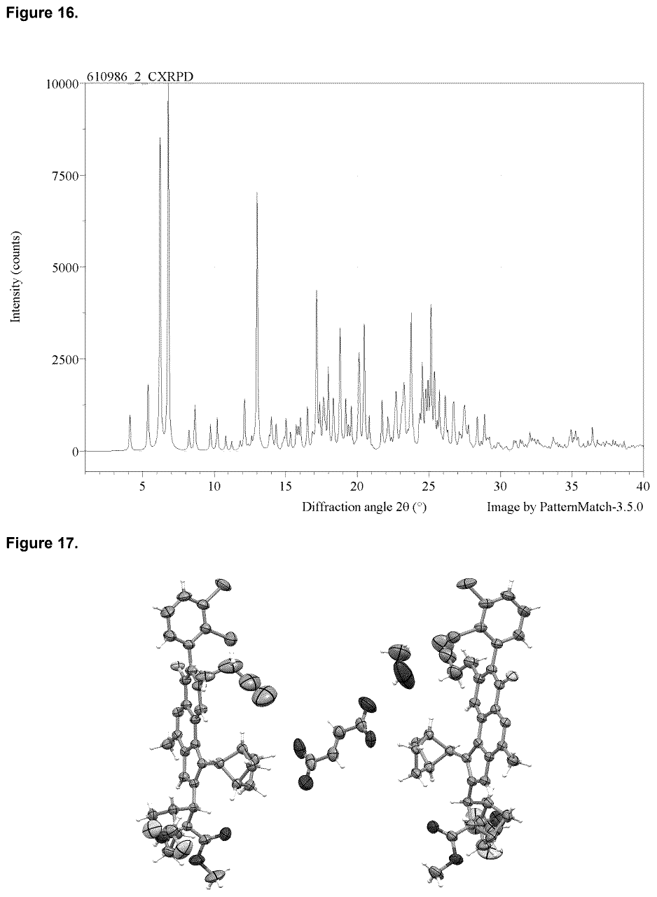

In another embodiment, the compound or pharmaceutically acceptable salt thereof is characterized by a calculated XRPD diffractogram substantially as depicted in FIG. 16.

The crystal system of Compound 1 hemifumarate hemimethanol is orthorhombic and the space group is P2₁2₁2. The cell parameters and calculated volume are: a=32.7815(6) Å, b=28.3055(6) Å, c=7.7571(2) Å, α=90°, β=90°, γ=90°, V=7197.8(3) Å³. The formula weight is 772.61 g mol⁻¹ with Z=8, resulting in a calculated density of 1.426 g cm⁻³.

### V. Methods of Treatment

Compound 1 of the present disclosure can inhibit the activity of the KRAS protein, particularly KRAS protein harboring a G12D mutation. Therefore, Compound 1, its crystalline forms, its crystalline salts and crystalline forms thereof, and formulations and dosage forms thereof as described in the present disclosure (collectively referred to as “compositions of the disclosure”) can be used to inhibit activity of KRAS, including KRAS harboring G12D, in a cell or in an individual or subject in need of inhibition of the enzyme by administering an inhibiting amount of the compound to the cell, individual, or subject. Compositions of the disclosure are therefore useful in treating diseases including cancers, in which KRAS particularly KRAS harboring a G12D mutation is implicated. Diseases in which KRAS is implicated include diseases associated with the expression or activity of KRAS, e.g., diseases in which abnormally proliferating cells (e.g., of a cancer) express KRAS. Diseases in which KRAS harboring a G12D mutation is implicated include diseases associated with the expression or activity of KRAS harboring a G12D mutation, e.g., diseases in which abnormally proliferating cells (e.g., of a cancer) express or comprise KRAS harboring a G12D mutation.

The cancer types in which KRAS, particularly KRAS harboring a G12D mutation, is implicated, and which can be treated using the compositions of the disclosure, include, but are not limited to: carcinomas (e.g., pancreatic, colorectal, lung, bladder, gastric, esophageal, breast, head and neck, cervical skin, thyroid); hematopoietic malignancies (e.g., myeloproliferative neoplasms (MPN), myelodysplastic syndrome (MDS), chronic and juvenile myelomonocytic leukemia (CMML and JMML), acute myeloid leukemia (AML), acute lymphocytic leukemia (ALL) and multiple myeloma (MM)); and other neoplasms (e.g., glioblastoma and sarcomas). In addition, KRAS mutations were found in acquired resistance to anti-EGFR therapy (Knickelbein et al., Genes & Cancer, 2015, 4-12). KRAS mutations were found in immunological and inflammatory disorders (Fernandez-Medarde, et al., Genes & Cancer, 2011, 344-58) such as Ras-associated lymphoproliferative disorder (RALD) or juvenile myelomonocytic leukemia (JMML) caused by somatic mutations of KRAS or NRAS.

Compositions of the disclosure are useful in the treatment of various diseases associated with abnormal expression or activity of KRAS. Compounds which inhibit KRAS will be useful in providing a means of preventing the growth or inducing apoptosis in tumors, or by inhibiting angiogenesis. It is therefore anticipated that compositions of the disclosure will prove useful in treating or preventing proliferative disorders such as cancers. In particular, tumors with activating mutants of receptor tyrosine kinases or upregulation of receptor tyrosine kinases may be particularly sensitive to the inhibitors.

In an aspect, provided herein is a method of inhibiting KRAS activity, said method comprising contacting Compound 1 with KRAS. In an embodiment, the contacting comprises administering a composition of the disclosure to a subject.

In an aspect, provided herein is a method of inhibiting a KRAS protein harboring a G12D mutation, said method comprising contacting KRAS with a Compound 1 In an embodiment, the contacting comprises administering a composition of the disclosure to a subject.

Thus, in an aspect, provided herein is a method of treating cancer in a subject in need thereof comprising administering to the subject a therapeutically effective amount of Compound 1, or a pharmaceutically acceptable salt thereof in the form of a composition of the disclosure. In an aspect, the cancer can be a cancer in which KRAS harboring a G12D mutation is implicated, such as cancers associated with the expression or activity of KRAS harboring a G12D mutation. Such cancers can include cancers in which abnormally proliferating cells (e.g., of a cancer) express or comprise KRAS harboring a G12D mutation.

In an embodiment, the cancer is associated with expression or activity of a KRAS protein. In another embodiment, the cancer is associated with expression or activity of a KRAS protein having a G12D mutation.

In another aspect, provided herein is a method for treating a cancer in a subject comprising identifying that the subject is in need of treatment of a cancer and that abnormally proliferating cells of the cancer comprise KRAS having a G12D mutation, and administering to the subject a therapeutically effective amount of Compound 1, or a pharmaceutically acceptable salt thereof in the form of a composition of the disclosure.

In an embodiment, the cancer is colorectal cancer. In another embodiment, the cancer is pancreatic cancer. In yet another embodiment, the cancer is pancreatic ductal cancer. In still another embodiment, the cancer is lung cancer. In another embodiment, the cancer is non-small cell lung cancer (NSCLC).

In another embodiment, the cancer is metastatic.

In still another embodiment, abnormally proliferating cells of the cancer comprise KRAS having a G12D mutation.

In another aspect, provided herein a is method of treating a disease or disorder associated with activity of KRAS, said method comprising administering to a subject in need thereof a therapeutically effective amount of a composition of the disclosure.

In an embodiment, the disease or disorder is an immunological or inflammatory disorder. In another embodiment, the immunological or inflammatory disorder is Ras-associated lymphoproliferative disorder or juvenile myelomonocytic leukemia caused by somatic mutations of KRAS.

In yet another aspect, provided herein is a method of treating a disease or disorder associated with abnormal activity of a KRAS protein harboring a G12D mutation, said method comprising administering to a subject in need thereof a therapeutically effective amount of a composition of the disclosure.

In another aspect, provided herein is a method of treating a disease or disorder associated with abnormal activity of a KRAS protein harboring a G12V mutation, said method comprising administering to a subject in need thereof a therapeutically effective amount of a composition of the disclosure.

In another aspect, provided herein is also a method of treating cancer in a subject in need thereof comprising administering to the subject a therapeutically effective amount of composition of the disclosure.

In still another aspect, provided herein is also a method of treating cancer in a subject in need thereof comprising administering to the subject a therapeutically effective amount of a composition of the disclosure wherein the cancer is characterized by with the presence or activity of a KRAS protein harboring a G12D mutation.

In yet another aspect, provided herein is a method for treating a cancer in a subject, said method comprising administering to the subject a therapeutically effective amount of composition of the disclosure.

In an embodiment, the cancer is selected from carcinomas, hematological cancers, sarcomas, and glioblastoma. In another embodiment, the hematological cancer is selected from myeloproliferative neoplasms, myelodysplastic syndrome, chronic and juvenile myelomonocytic leukemia, acute myeloid leukemia, acute lymphocytic leukemia, and multiple myeloma. In yet another embodiment, the carcinoma is selected from pancreatic, colorectal, lung, bladder, gastric, esophageal, breast, head and neck, cervical, skin, and thyroid.

In an aspect, provided herein is a method for treating a disease or disorder associated with activity of KRAS interaction or a mutant thereof, in a subject in need thereof, comprising the step of administering to the subject a composition of the disclosure, in combination with another therapy or therapeutic agent as described herein.

In an embodiment, the cancer is selected from hematological cancers, sarcomas, lung cancers, gastrointestinal cancers, genitourinary tract cancers, liver cancers, bone cancers, nervous system cancers, gynecological cancers, and skin cancers.

In another embodiment, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet another embodiment, the lung cancer is non-small cell lung cancer (NSCLC). In still another embodiment, the lung cancer is adenocarcinoma.

In an embodiment, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In an embodiment, the gastrointestinal cancer is colorectal cancer.

In another embodiment, the cancer is a carcinoma. In yet another embodiment, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still another embodiment, the cancer is a hematopoietic malignancy. In an embodiment, the hematopoietic malignancy is selected from multiple myeloma, acute myelogenous leukemia, and myeloproliferative neoplasms.

In another embodiment, the cancer is a neoplasm. In yet another embodiment, the neoplasm is glioblastoma or sarcomas.

In certain embodiments, the disclosure provides a method for treating a KRAS-mediated disorder in a subject in need thereof, comprising the step of administering to said subject a composition of the disclosure, or a pharmaceutically acceptable composition thereof.

In some embodiments, diseases and indications that are treatable using the compositions of the disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET), 8p11 myeloproliferative syndrome, myelodysplasia syndrome (MDS), T-cell acute lymphoblastic lymphoma (T-ALL), multiple myeloma, cutaneous T-cell lymphoma, adult T-cell leukemia, Waldenstrom's Macroglubulinemia, hairy cell lymphoma, marginal zone lymphoma, chronic myelogenic lymphoma and Burkitt's lymphoma. Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, lymphosarcoma, leiomyosarcoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, mesothelioma, pavicellular and non-pavicellular carcinoma, bronchial adenoma and pleuropulmonary blastoma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (exocrine pancreatic carcinoma, ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, gall bladder cancer and anal cancer.

Exemplary genitourinary tract cancers include cancers of the kidney (adenocarcinoma, Wilm's tumor [nephroblastoma], renal cell carcinoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma) and urothelial carcinoma.

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors, neuro-ectodermal tumors), and spinal cord (neurofibroma, meningioma, glioma, sarcoma), neuroblastoma, Lhermitte-Duclos disease and pineal tumors.

Exemplary gynecological cancers include cancers of the breast (ductal carcinoma, lobular carcinoma, breast sarcoma, triple-negative breast cancer, HER2-positive breast cancer, inflammatory breast cancer, papillary carcinoma), uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers, tumors of the eye, tumors of the lips and mouth and squamous head and neck cancer.

The compositions of the disclosure can also be useful in the inhibition of tumor metastases.

In addition to oncogenic neoplasms, the compositions of the disclosure are useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes. In some embodiments, the present disclosure provides a method for treating a subject suffering from a skeletal and chondrocyte disorder.

In an embodiment, the cancer is selected from hematological cancers, sarcomas, lung cancers, gastrointestinal cancers, genitourinary tract cancers, liver cancers, bone cancers, nervous system cancers, gynecological cancers, and skin cancers.

In another embodiment, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet another embodiment, the lung cancer is non-small cell lung cancer (NSCLC). In still another embodiment, the lung cancer is adenocarcinoma.

In an embodiment, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In an embodiment, the gastrointestinal cancer is colorectal cancer.

In another embodiment, the cancer is a carcinoma. In yet another embodiment, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still another embodiment, the cancer is a hematopoietic malignancy. In an embodiment, the hematopoietic malignancy is selected from multiple myeloma, acute myelogenous leukemia, and myeloproliferative neoplasms.

In another embodiment, the cancer is a neoplasm. In yet another embodiment, the neoplasm is glioblastoma or sarcomas.

In an embodiment, the cancer is selected from the group consisting of hematological cancers, sarcomas, lung cancers, gastrointestinal cancers, genitourinary tract cancers, liver cancers, bone cancers, nervous system cancers, gynecological cancers, and skin cancers.

In an embodiment, the cancer is selected from the group consisting of pancreatic cancer, cervical cancer, colon cancer, ovarian cancer, breast cancer, pancreatic cancer, carcinoma, and adenocarcinoma.

In another embodiment, the cancer is pancreatic cancer. In yet another embodiment, the cancer is a solid tumor.

In one embodiment of the methods described herein, the subject is human.

### VI. Pharmaceutical Compositions

Compound 1, its crystalline forms, its crystalline salts and crystalline forms thereof, as described in the present disclosure can be administered in pharmaceutical compositions comprising a crystalline form disclosed herein and at least one pharmaceutically acceptable carrier or excipient.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: The Science & Practice of Pharmacy by Alfonso Gennaro, 20^{th} ed., Lippencott Williams & Wilkins (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

In an aspect, provided herein is a pharmaceutical composition comprising:
a) compound 1, its crystalline forms, its crystalline salts and crystalline forms thereof, and formulations and dosage forms thereof as described in the present disclosure;
b) a disintegrant;
c) a binder;
d) an anti-caking agent; and
e) a lubricant.

In an embodiment, the composition comprises two lubricants. In an embodiment, the composition further comprises f) an additive. In an embodiment, the composition further comprises f) a second lubricant. In an embodiment, the composition comprises two binders.

In another embodiment, the crystalline form of a) is present in a dose of between about 25 mg and 200 mg. In yet another embodiment, the crystalline form of a) is present in a dose of about 25 mg. In still another embodiment, the crystalline form of a) is present in a dose of about 100 mg. In an embodiment, the crystalline form of a) is present in a dose of about 200 mg. The foregoing doses refer to the free base equivalent of Compound 1.

In another embodiment, disintegrant b) is sodium starch glycolate.

In yet another embodiment, binder c) is microcrystalline cellulose. In another embodiment, binder c) is lactose anhydrous.

In still another embodiment, anti-caking agent d) is colloidal silicon dioxide.

In an embodiment, lubricant e) is sodium stearyl fumarate.

In another embodiment, additive f) is magnesium stearate. In another embodiment, second lubricant f) is magnesium stearate.

In another aspect, provided herein is a pharmaceutical composition comprising:
a) the crystalline form of the present disclosure;
b) sodium starch glycolate;
c) microcrystalline cellulose and lactose anhydrous;
d) colloidal silicon dioxide;
e) sodium stearyl fumarate; and
f) magnesium stearate.

### VII Administration/Dosage/Formulations

In another aspect, provided herein is a pharmaceutical composition comprising a crystalline form provided herein, together with a pharmaceutically acceptable carrier.

Actual dosage levels of the active ingredients in the pharmaceutical compositions discussed herein may be varied to obtain an amount of the active ingredient that is effective to achieve the desired therapeutic response for a particular subject, composition, and mode of administration, without being toxic to the subject.

In particular, the selected dosage level will depend upon a variety of factors including the activity of the particular compound employed, the time of administration, the rate of excretion of the compound, the duration of the treatment, other drugs, compounds or materials used in combination with the crystalline form, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed crystalline form at levels lower than that required in order to achieve the desired therapeutic effect and gradually increase the dosage until the desired effect is achieved.

In particular embodiments, it is especially advantageous to formulate the crystalline form in dosage unit form for ease of administration and uniformity of dosage.

“Dosage unit form,” as used herein, refers to physically discrete units suited as unitary dosages for the subjects to be treated; each unit containing a predetermined quantity of the disclosed compound calculated to produce the desired therapeutic effect in association with the required pharmaceutical vehicle. The dosage unit forms of the crystalline form disclosed herein are dictated by and directly dependent on (a) the unique characteristics of the disclosed compound and the particular therapeutic effect to be achieved, and (b) the limitations inherent in the art of compounding/formulating such a disclosed compound for the treatment of cancer in a subject.

As used herein, the term “free base equivalent” refers to the amount of the active agent (e.g., Compound 1) present in the active agent or pharmaceutically acceptable salt thereof. Stated alternatively, the term “free base equivalent” means either an amount of Compound 1 free base, or the equivalent amount of Compound 1 free base that is provided by a salt of said compound. The dosages provided herein refer to the free base equivalent of Compound 1.

In one embodiment, the crystalline form provided herein is formulated using one or more pharmaceutically acceptable excipients or carriers. In one embodiment, the pharmaceutical compositions comprise a therapeutically effective amount of the disclosed crystalline form and a pharmaceutically acceptable carrier.

In an aspect, provided herein is a dosage form comprising Compound 1 or pharmaceutically acceptable salt thereof or a pharmaceutical composition disclosed herein.

In an embodiment, the dosage form is in the form of a tablet. In another embodiment, the compound or pharmaceutically acceptable salt thereof is present in an amount that provides a dose of about 25 mg to about 200 mg of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile per dosage form.

In another embodiment, the compound or pharmaceutically acceptable salt thereof is present in an amount that provides a dose of about 25 mg of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile per dosage form.

In yet another embodiment, the compound or pharmaceutically acceptable salt thereof a) is present in an amount that provides a dose of about 100 mg of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile per dosage form.

In still another embodiment, the compound or pharmaceutically acceptable salt thereof a) is present in an amount that provides a dose of about 200 mg of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile per dosage form.

In some embodiments, the dose of a disclosed crystalline form is from about 1 mg to about 1,000 mg. In some embodiments, the amount of Compound 1, its crystalline forms, its crystalline salts and crystalline forms thereof, and formulations and dosage forms thereof as described in the present disclosure are included in an amount that provides a dose Compound 1 (calculated as the amount equivalent to the amount of free base of Compound 1) that is less than about 1,000 mg, or less than about 800 mg, or less than about 600 mg, or less than about 500 mg, or less than about 300 mg, or less than about 200 mg, or less than about 100 mg, or less than about 50 mg, or less than about 20 mg, or less than about 10 mg. For example, a dose is about 10 mg, 20 mg, 25 mg, 30 mg, 40 mg, 50 mg, 60 mg, 70 mg, 80 mg, 90 mg, 100 mg, 120 mg, 140 mg, 160 mg, 180 mg, 200 mg, 220 mg, 240 mg, 260 mg, 280 mg, 300 mg, 350 mg, 400 mg, 450 mg, 500 mg, 550 mg, or about 600 mg.

Routes of administration of any of the compositions disclosed herein include oral, nasal, rectal, intravaginal, parenteral, buccal, sublingual or topical. The compound for use provided herein may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example, an inert diluent such as lactose; granulating and disintegrating agents such as cornstarch; binding agents such as starch; and lubricating agents such as magnesium stearate. The tablets may be uncoated, or they may be coated by known techniques for elegance or to delay the release of the active ingredients. Formulations for oral use may also be presented as hard gelatin capsules wherein the active ingredient is mixed with an inert diluent.

For parenteral administration, the disclosed compound may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize or be able to ascertain using no more than routine experimentation, numerous equivalents to the specific procedures, embodiments, claims, and examples described herein. Such equivalents are within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The disclosure is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

X-Ray Powder Diffraction (XRPD) was performed using a Bruker D8 Advance ECO X-ray Powder Diffractometer (XRPD) instrument. The general experimental procedures for XRPD were: (1) X-ray radiation from copper at 1.5418 Å and LYNXEYE™ detector; (2) X-ray power at 40 kV, 25 mA; and (3) the sample powder was dispersed on a zero-background sample holder. The general measurement conditions for XRPD were: Start Angle 3 degrees; Stop Angle 30 degrees; Sampling 0.015 degrees; and Scan speed 2 degree/min.

Differential Scanning Calorimetry (DSC) was performed using a TA Instruments Differential Scanning Calorimeter, Discovery DSC2500 with autosampler. The DSC instrument conditions were as follows: 20-300° C. at 10° C./min.; T_{zero} aluminum sample pan and lid; and nitrogen gas flow at 50 mL/min.

Thermogravimetric Analysis (TGA) was performed using a TA Instruments Thermogravimetric Analyzer, Discovery TGA5500 with autosampler. The general experimental conditions for TGA were ramp from 25° C. to 300° C. at 10° C./min.; nitrogen purge gas flow at 25 mL/min; platinum sample holder.

### Example 1: Synthesis of methyl (1R,3R,4R,5S)-3-((R,)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 1)

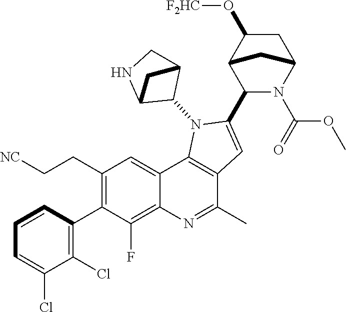

Compound 1 can be prepared by the process shown in the following scheme:

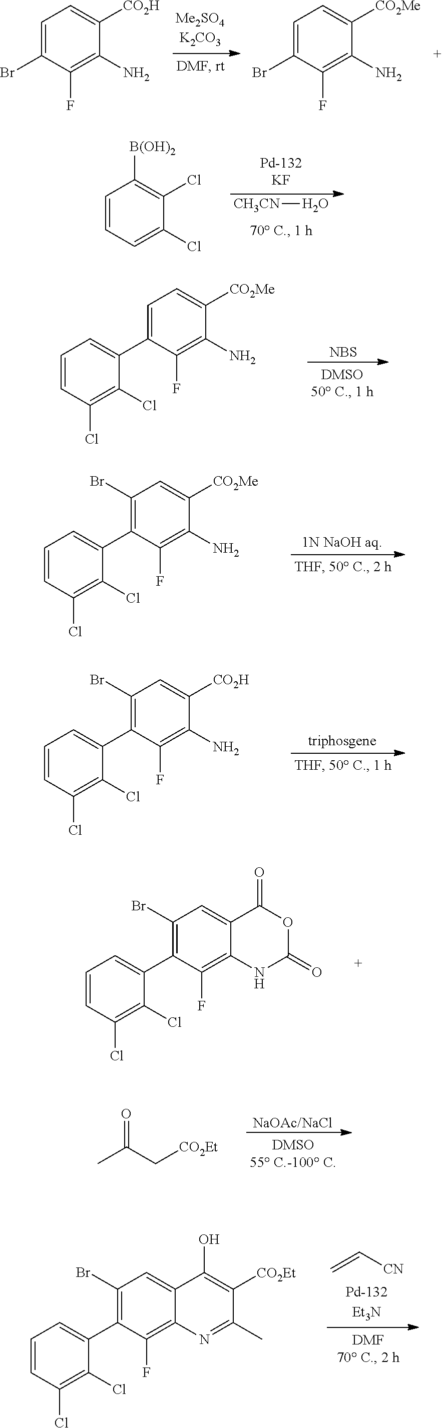

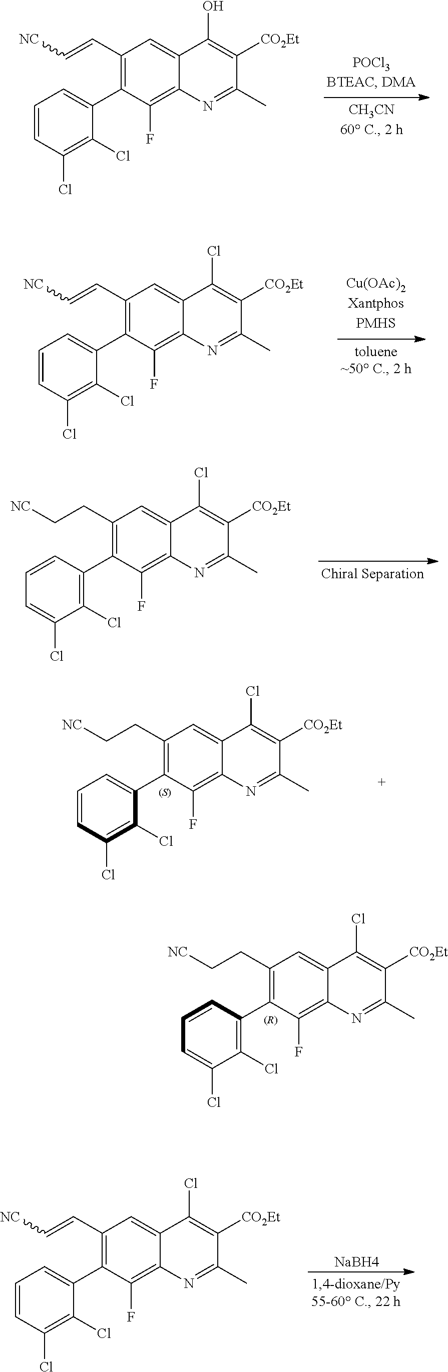

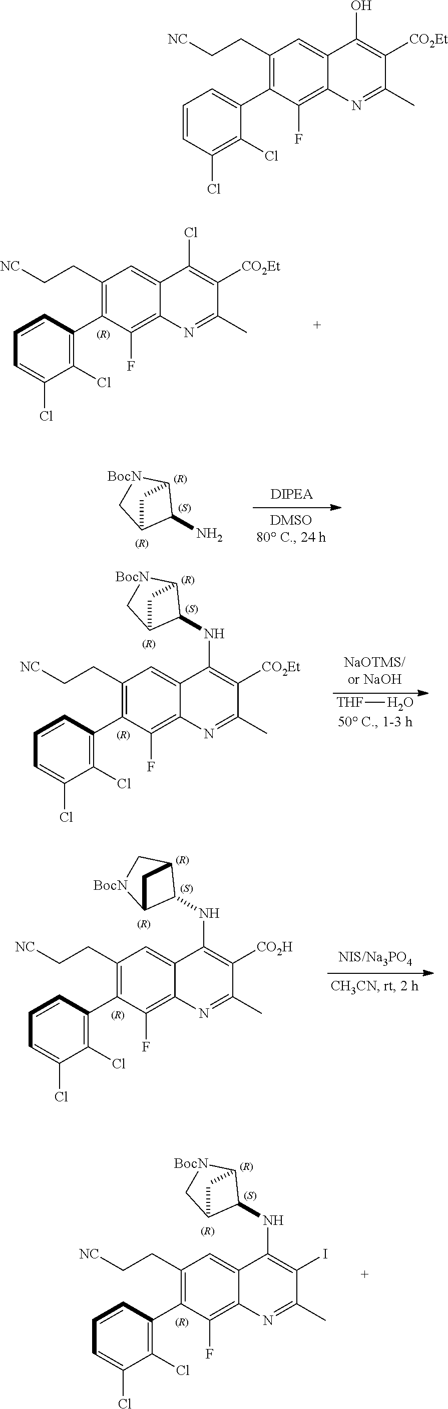

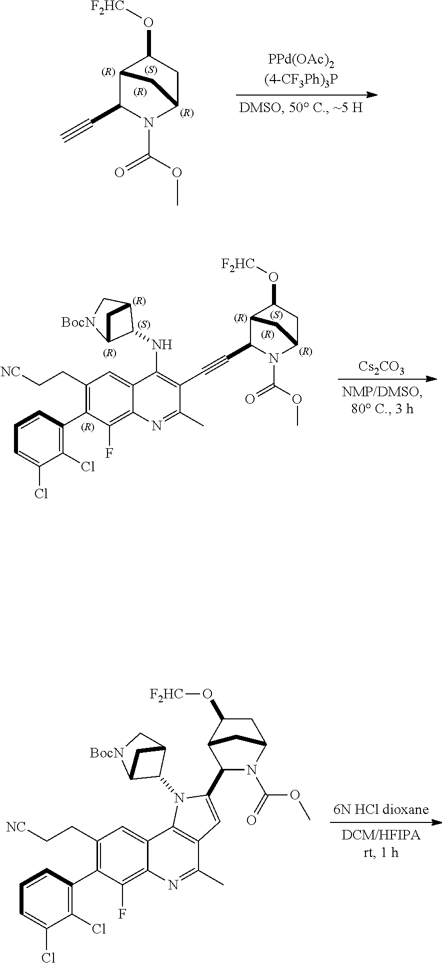

### Step 1. Methyl 2-amino-4-bromo-3-fluorobenzoate

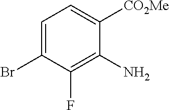 [SMILES: COC(=O)c1ccc(Br)c(F)c1N]

Dimethyl sulfate (823 g, 6.53 mol) was added to a mixture of 2-amino-4-bromo-3-fluorobenzoic acid (1500 g, 6.22 mol) and K₂CO₃ (945 g, 6.84 mol) in DMF or 1,4-dioxane (6 L) at 5-50° C. After the addition, the mixture was stirred at r.t. for 2 h to complete the reaction. Water (7.5 L) was gradually added to the reaction mixture to precipitate the product. After the water addition, the mixture was stirred at r.t. for 1 h. The solids were isolated by filtration and the wet cake was washed with water (3×1.5 L). The solids were dried under vacuum at about 50° C. overnight to give the title compound (1530 g, 99% yield). LCMS calc. for C₈H₇BrFNO₂: 246.96; Found: 248 (M+H⁺). ¹H NMR (400 MHz, DMSO-d₆) δ 7.49 (dd, J=8.8, 1.7 Hz, 1H), 6.87-6.77 (m, 3H), 3.82 (s, 3H). ¹⁹F NMR (376 MHz, DMSO-d₆) δ −127.24.

### Step 2. Methyl 3-amino-2′,3′-dichloro-2-fluoro-[1,1′-biphenyl]-4-carboxylate

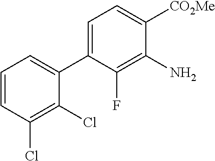 [SMILES: COC(=O)c1ccc(-c2cccc(Cl)c2Cl)c(F)c1N]

Bis(di-tert-butyl(4-dimethylaminophenyl)phosphine)dichloropalladium(II) (Pd-132) (8.12 g, 0.011 mol) was added to a mixture of methyl 2-amino-4-bromo-3-fluorobenzoate (1420 g, 5.72 mol), 2,3-dichlorophenylboronic acid (1226 g, 6.3 mol) and KF (732 g, 12.6 mol) in MeCN (6 L) and water (1.5 L). The mixture was degassed and refilled with N₂ and heated to 70° C. for 1 h to complete the reaction. Water (6 L) was added to the reaction mixture at 50° C. The mixture was cooled to r.t. and stirred for 1 h. The solids were isolated by filtration and the wet cake was washed with 50% MeCN in water (2×2 L) and water (2×2 L). The solids were dried under vacuum at about 50° C. overnight to give the title compound (1700 g, 94% yield). LCMS calc. for C₁₄H₉Cl₂FNO₂: 313.01; Found: 314 (M+H⁺). ¹H NMR (400 MHz, DMSO-d₆) δ 7.74 (dd, J=8.0, 1.6 Hz, 1H), 7.64 (dd, J=8.4, 1.4 Hz, 1H), 7.48 (t, J=7.9 Hz, 1H), 7.40 (dd, J=7.9, 1.6 Hz, 1H), 6.70 (s(b), 2H), 6.51 (dd, J=8.3, 6.6 Hz, 1H), 3.86 (s, 3H). ¹⁹F NMR (376 MHz, DMSO-d₆) δ −134.70.

### Step 3. Methyl 3-amino-6-bromo-2′,3′-dichloro-2-fluoro-[1,1′-biphenyl]-4-carboxylate

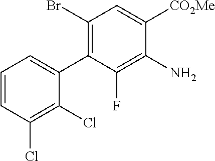 [SMILES: COC(=O)c1cc(Br)c(-c2cccc(Cl)c2Cl)c(F)c1N]

N-Bromosuccinimide (684 g, 3.84 mol) was added to a solution of methyl 3-amino-2′,3′-dichloro-2-fluoro-[1,1′-biphenyl]-4-carboxylate (1150 g, 3.66 mol) in MeCN (5.75 L) at 50-66° C. After the reaction completion, the MeCN (3 L) was removed in vacuo. Water (5.75 L) was added to the concentrated mixture and the resulting mixture was stirred at r.t. for 2-3 h. The solids were isolated by filtration and the wet cake was washed with water to give methyl 3-amino-6-bromo-2′,3′-dichloro-2-fluoro-[1,1′-biphenyl]-4-carboxylate. LCMS calc. for C₁₄H₉BrFCl₂NO₂: 390.92; Found: 391 (M+H). ¹H NMR (400 MHz, DMSO-d₆) δ 7.86 (d, J=1.7 Hz, 1H), 7.79 (dd, J=8.1, 1.5 Hz, 1H), 7.52 (t, J=7.9 Hz, 1H), 7.40 (dd, J=7.7, 1.5 Hz, 1H), 6.83 (s(b), 2H), 3.87 (s, 3H). ¹⁹F NMR (376 MHz, DMSO-d₆) δ −128.19.

### Step 4. 3-Amino-6-bromo-2′,3′-dichloro-2-fluoro-[1,1′-biphenyl]-4-carboxylic acid

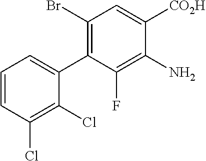 [SMILES: Nc1c(C(=O)O)cc(Br)c(-c2cccc(Cl)c2Cl)c1F]

The wet cake of methyl 3-amino-6-bromo-2′,3′-dichloro-2-fluoro-[1,1′-biphenyl]-4-carboxylate was dissolved in THF (3 L) and MeOH (1.5 L). Aq. NaOH (1.5 M; 5 L) was added to the solution and the mixture was stirred at about 50° C. for 2 h to complete the saponification reaction. Aq. HCl (1.5 M) was gradually added to the mixture to adjust the pH to 3-4 and the mixture was stirred at r.t. for 1 h. The solids were isolated by filtration and the wet cake was washed with water (3×1.2 L). The solids were dried under vacuum at about 50° C. overnight to give the title compound (1354 g, 97.5% yield over two steps). LCMS calc. for C₁₃H₇BrCl₂FNO₂: 376.90; Found: 378 (M+H⁺). ¹H NMR (400 MHz, DMSO-d₆) δ 7.85 (d, J=1.7 Hz, 1H), 7.78 (dd, J=8.1, 1.5 Hz, 1H), 7.52 (t, J=7.9 Hz, 1H), 7.39 (dd, J=7.9, 1.5 Hz, 1H), 6.88. ¹⁹F NMR (376 MHz, DMSO-d₆) δ −128.95.

### Step 5. 6-Bromo-7-(2,3-dichlorophenyl)-8-fluoro-2H-benzo[d][1,3]oxazine-2,4(1H)-dione

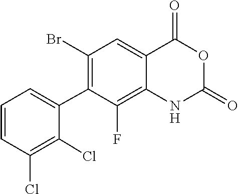 [SMILES: O=c1[nH]c2c(F)c(-c3cccc(Cl)c3Cl)c(Br)cc2c(=O)o1]

Triphosgene (500 g, 1.65 mol) in THF (500 mL) was added to the solution of 3-amino-6-bromo-2′,3′-dichloro-2-fluoro-[1,1′-biphenyl]-4-carboxylic acid (1254 g, 3.31 mol) in THF (4 L) at 60° C. and the mixture was stirred for 1 h to complete the reaction. The mixture was cooled to 35° C. and n-heptane (10 L) was slowly charged to precipitate the product. The mixture was cooled to r.t. and stirred for 1 h. The solids were isolated by filtration and washed with n-heptane (2×1 L). The wet cake was dried under vacuum at about 50° C. overnight to give the title compound (1385 g, quantitative yield). LCMS calc. for C₁₄H₅BrCl₂FNO₃: 402.88; Found: 404 (M+H⁺). ¹H NMR (400 MHz, DMSO-d₆) δ 12.24 (s, 1H), 8.10 (d, J=1.5 Hz, 1H), 7.85 (dd, J=8.1, 1.5 Hz, 1H), 7.58 (t, J=7.9 Hz, 1H), 7.43 (dd, J=7.7, 1.5 Hz, 1H). ¹⁹F NMR (376 MHz, DMSO-d₆) δ −123.98.

### Step 6. Ethyl 6-bromo-7-(2,3-dichlorophenyl)-8-fluoro-4-hydroxy-2-methylquinoline-3-carboxylate

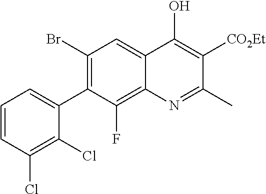 [SMILES: CCOC(=O)c1c(C)nc2c(F)c(-c3cccc(Cl)c3Cl)c(Br)cc2c1O]

A mixture of 6-bromo-7-(2,3-dichlorophenyl)-8-fluoro-2H-benzo[d][1,3]oxazine-2,4(1H)-dione (1078 g, 2.66 mol), ethyl acetoacetate (693 g, 5.32 mol), NaOAc (393 g, 4.79 mol) and NaCl (933 g. 16 mol) in DMSO (5 L) was heated to 50-60° C. for 5 h. The mixture was heated to 100° C. and stirred for 1 h to complete the reaction. The mixture was cooled to about 60° C. and water (10 L) was gradually added to precipitate the product. The mixture was cooled to r.t. and stirred for 1 h. The solids were isolated by filtration and the wet cake was washed with water (2×2 L). The wet solids were dried under vacuum at about 50° C. overnight to give the title compound (1145 g, 91% yield). LCMS calc. for C₁₉H₁₃BrCl₂FNO₂: 470.94; Found: 472 (M+H⁺). ¹H NMR (400 MHz, DMSO-d₆) b 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 6a. Ethyl 6-bromo-7-(2,3-dichlorophenyl)-8-fluoro-4-hydroxy-2-methylquinoline-3-carboxylate

The title compound can alternatively be prepared by the following process. A solution of methyl 3-amino-2′,3′-dichloro-2-fluoro-[1,1′-biphenyl]-4-carboxylate (100 g, 0.254 mol), ethyl acetoacetate (33.1 g, 0.51 mol) and p-TsOH (2,2 g, 0.013 mol) in xylene (1 L) was refluxed for 5 h to azeotropically remove water. NaOEt (26 g, 0.381 mol) was added to the mixture and the mixture was refluxed for another 5 h. The mixture was cooled to r.t. and poured into dilute HCl pH=6-7. The organic phase was separated, and the aqueous phase was extracted with EtOAc. The combined organic phases were concentrated, and the product was purified via FCC and eluted with EtOAc and heptane (0-30%) to give the title compound (65 g, 54%). LCMS calc. for C₁₉H₁₃BrCl₂FNO₃: 470.91; Found: 472 (M+H⁺). ¹H NMR (400 MHz, DMSO-d₆) δ 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 7. Ethyl 6-(2-cyanovinyl)-7-(2,3-dichlorophenyl)-8-fluoro-4-hydroxy-2-methylquinoline-3-carboxylate

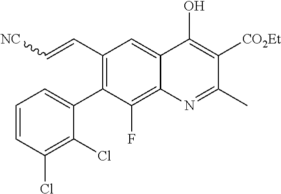 [SMILES: CCOC(=O)c1c(C)nc2c(F)c(-c3cccc(Cl)c3Cl)c(C=CC#N)cc2c1O]

A mixture of ethyl 6-bromo-7-(2,3-dichlorophenyl)-8-fluoro-4-hydroxy-2-methylquinoline-3-carboxylate (246 g, 0.52 mol), acrylonitrile (69 g, 1.3 mol), NEt₃ (156 g, 1.56 mol) and bis(di-tert-butyl)-(4-dimethylaminophenyl)phosphine)dichloridopalladium (II) (Pd-132) (14.7 g, 0.02 mol) in DMF (1.5 L) was heated to 85° C. for about 5 h to complete the reaction. The mixture was cooled to 50° C. and water (1 L) was gradually added. The mixture was cooled to r.t. and aq. HCl (1 M) was added to adjust the pH to 5-6. The solids were isolated by filtration and the wet cake was washed with water (2×500 mL). The wet solids were dissolved in MeOH (1 L) and DCM (9 L). To the solution was added NaHSO₃ (186 g, 1.8 mol) and water (4 L). The mixture was stirred at r.t. for 1 h and the aqueous phase was separated and discarded. The organic phase was washed with water (2×2 L). Activated charcoal (150 g) was added to the organic solution and the mixture was stirred at r.t. for 1 h. The mixture was filtered over a diatomaceous earth bed and the bed was rinsed with DCM (2 L). The organic solution was concentrated to about 1 L and heptane (3.5 L) was gradually added to precipitate the product. The solids were isolated by filtration and washed with heptane (2×2 L). The wet solids were dried under vacuum at about 50° C. overnight to give the title compound (210 g, 90% yield). LCMS calc. for C₂₂H₁₅Cl₂FNO₃: 444.04; Found: 445 (M+H⁺). ¹H-NMR (400 MHz, DMSO-d₆) (cis and trans mixture): 512.05 (s, 1H), 8.64 (s, OH), 8.39 (s, 1H), 7.86 (td, J=7.7, 1.5 Hz, 1H), 7.63-7.53 (m, 1H), 7.47 (td, J=7.5, 1.6 Hz, 1H), 7.04 (d, J=16.5 Hz, 1H), 6.88 (d, J=11.9 Hz, OH), 6.55 (d, J=16.6 Hz, 1H), 5.91 (d, J=12.0 Hz, OH), 4.29 (q, J=7.1 Hz, 2H), 2.47 (d, J=5.0 Hz, 4H), 1.30 (td, J=7.1, 3.2 Hz, 4H).

### Step 8. Ethyl 6-(2-cyanoethyl)-7-(2,3-dichlorophenyl)-8-fluoro-4-hydroxy-2-methylquinoline-3-carboxylate

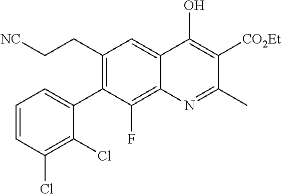 [SMILES: CCOC(=O)c1c(C)nc2c(F)c(-c3cccc(Cl)c3Cl)c(CCC#N)cc2c1O]

A mixture of ethyl 6-(2-cyanovinyl)-7-(2,3-dichlorophenyl)-8-fluoro-4-hydroxyl-2-methylquinoline-3-carboxylate (155 g, 348 mmol), pyridine (450 mL) and 1,4-dioxane (450 mL) was heated to 50-60° C. to give a homogenous solution. To the solution was added NaBH₄ (65.8 g, 1741 mmol) in portions at 50-60° C. The resulting mixture was stirred for 22 h at 50-60° C. to complete the reduction. After cooling to about 15° C., EtOAc (950 mL) was added to the reaction mixture. Concentrated HCl was gradually added to the mixture to adjust the aqueous phase pH to 1-2. The organic phase was separated, and the aqueous phase was extracted with EtOAc (500 mL). The combined EtOAc phase was washed with aq. HCl (1 M, 500 mL), water (2×500 mL), 10% brine (300 mL) and dried over Na₂SO₄ (75 g). The solution was concentrated, and the residue was purified by FCC (0-20% MeOH in DCM) to give the title compound (117.8 g, 76%). LCMS calc. for C₂₂H₁₇Cl₂FN₂O₃: 446.06; Found: 447 (M+H⁺). ¹H NMR (400 MHz, DMSO-d₆) δ 11.87 (s, 1H), 8.00 (s, 1H), 7.84 (dd, J=7.9, 1.7 Hz, 1H), 7.71-7.48 (m, 2H), 4.28 (q, J=7.1 Hz, 2H), 2.79 (ddd, J=11.7, 7.4, 3.7 Hz, 1H), 2.73-2.59 (m, 3H), 2.46 (s, 3H), 1.30 (t, J=7.1 Hz, 3H).

### Step 9. Ethyl 4-chloro-6-(2-cyanoethyl)-7-(2,3-dichlorophenyl)-8-fluoro-2-methylquinoline-3-carboxylate

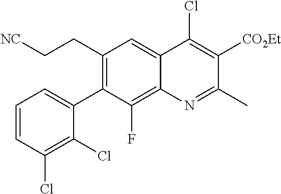 [SMILES: CCOC(=O)c1c(C)nc2c(F)c(-c3cccc(Cl)c3Cl)c(CCC#N)cc2c1Cl]

A mixture of ethyl 6-(2-cyanoethyl)-7-(2,3-dichlorophenyl)-8-fluoro-4-hydroxy-2-methylquinoline-3-carboxylate (60 g, 134 mmol), benzyltriethylammonium chloride (31 g, 135 mmol), N,N-dimethylaniline (49.1 g, 405 mmol) in MeCN (300 mL) was added POCl₃ (62 g, 405 mmol) at below 20° C. The mixture was heated to 60° C. for 1 h to complete the reaction. The mixture was cooled to r.t. and pooled into ice-water (900 mL) at a temperature below 20° C. Product precipitated out during the aqueous quench. The mixture was stirred at r.t. for more than 5 h. The solids were isolated by filtration and the wet cake was washed with 10% MeCN in water (2×150 mL). The wet solids were dried under vacuum at about 50° C. overnight to give the title compound (57 g, 90% yield). LCMS calc. for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 10. Ethyl 4-chloro-6-(2-cyanovinyl)-7-(2,3-dichlorophenyl)-8-fluoro-2-methylquinoline-3-carboxylate

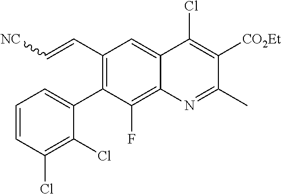 [SMILES: CCOC(=O)c1c(C)nc2c(F)c(-c3cccc(Cl)c3Cl)c(C=CC#N)cc2c1Cl]

A mixture of ethyl 6-(2-cyanovinyl)-7-(2,3-dichlorophenyl)-8-fluoro-4-hydroxy-2-methylquinoline-3-carboxylate (600 g, 1.35 mol), benzyltriethylammonium chloride (307 g, 1.35 mol), N,N-diethylaniline (603 g, 4.04 mol) in MeCN (3 L) was added POCl₃ (389.7 g, 4.04 mol) at below 20° C. The mixture was heated to 60° C. for 1 h to complete the reaction. The mixture was cooled to r.t. and pooled into ice-water (9 L) at a temperature below 20° C. Product precipitated during the aqueous quench. The mixture was stirred at r.t. for more than 5 h. The solids were isolated by filtration and the wet cake was washed with 10% MeCN in water (2×1.5 L). The wet solids were dried under vacuum at about 50° C. overnight to give the title compound (563 g, 90% yield). LCMS calc. for C₂₂H₁₄Cl₃FN₂O₂: 462.01; Found: 463 (M+H⁺). ¹H-NMR (400 MHz, DMSO-d₆) (mixture of cis and trans isomers) b 8.72 (s, 0.3H), 8.51 (s, 1H), 7.87 (ddd, J=7.3, 5.6, 1.5 Hz, 1.3H), 7.64-7.46 (m, 3H), 7.21 (d, J=16.5 Hz, 1H), 7.05 (d, J=11.9 Hz, 0.3H), 6.73 (d, J=16.5 Hz, 1H), 6.08 (d, J=11.9 Hz, 0.3H), 4.53 (qd, J=7.1, 2.0 Hz, 2H), 2.72 (d, J=7.4 Hz, 4H), 1.41 (t, J=7.1 Hz, 4H).

### Step 11. Ethyl 4-chloro-6-(2-cyanoethyl)-7-(2,3-dichlorophenyl)-8-fluoro-2-methylquinoline-3-carboxylate

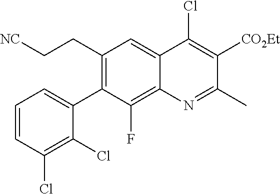 [SMILES: CCOC(=O)c1c(C)nc2c(F)c(-c3cccc(Cl)c3Cl)c(CCC#N)cc2c1Cl]

A mixture of ethyl 4-chloro-6-(2-cyanovinyl)-7-(2,3-dichlorophenyl)-8-fluoro-2-methylquinoline-3-carboxylate (528 g, 1.14 mol) and PMHS (411 g, 6.83 mol) in toluene (1.8 L) was stirred at about 50° C. In another 2-L flask, a mixture of diacetoxycopper hydrate (4.1 g, 0.02 mol), (9,9-dimethyl-9H-xanthene-4,5-diyl)bis(diphenylphosphane) (13.58 g, 0.023 mol) in toluene (300 mL) and t-BuOH (483 g, 6.52 mol) was stirred for 1-2 h to form a solution. The copper acetate solution was slowly added to the solution of ethyl 4-chloro-6-(2-cyanovinyl)-7-(2,3-dichlorophenyl)-8-fluoro-2-methylquinoline-3-carboxylate and PMHS in toluene at 50-60° C. to complete the reduction. The reaction mixture was concentrated under vacuum distillation to about 2 L. To the 2 L residue was added heptane (8 L) at about 50° C. for 1 h. The mixture was cooled to r.t. and stirred overnight. The solids were isolated by filtration and the wet cake was washed with heptane (2×1.2 L). The wet cake and silica gel (260 g) in DCM (2.7 L) were stirred for 1 h. The mixture was filtered over a silica gel bed (260 g) and the silica gel bed was rinsed with DCM (4 L) until the eluent was almost colorless. The DCM was removed. DCM (140 mL) and MTBE (260 mL) were added to the residue. The solids were isolated by filtration and the wet cake was washed with MTBE (2×1.2 L). The wet solids were dried under vacuum at about 50° C. overnight to give the title compound (476 g, 90% yield). LCMS calc. for C₂₂H₁₆Cl₃FN₂O₂: 464.03; Found: 465 (M+H⁺). ¹H-NMR (400 MHz, DMSO-d₆) b 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 12. Ethyl (Rₐ)-4-chloro-6-(2-cyanoethyl)-7-(2,3-dichlorophenyl)-8-fluoro-2-methylquinoline-3-carboxylate and ethyl (Sₐ)-4-chloro-6-(2-cyanoethyl)-7-(2,3-dichlorophenyl)-8-fluoro-2-methylquinoline-3-carboxylate

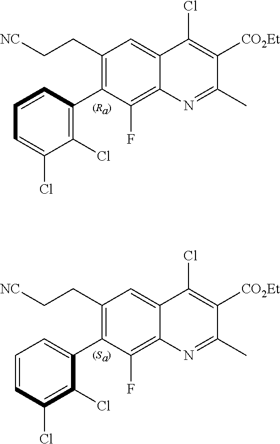

The racemic ethyl 4-chloro-6-(2-cyanoethyl)-7-(2,3-dichlorophenyl)-8-fluoro-2-methylquinoline-3-carboxylate was subject to chiral separation (CHIRALPAK® IB N, MTBE as eluent) to give both ethyl (Rₐ)-4-chloro-6-(2-cyanoethyl)-7-(2,3-dichlorophenyl)-8-fluoro-2-methylquinoline-3-carboxylate and ethyl (Sₐ)-4-chloro-6-(2-cyanoethyl)-7-(2,3-dichlorophenyl)-8-fluoro-2-methylquinoline-3-carboxylate. LCMS calc. for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 13. Ethyl 4-chloro-6-(2-cyanoethyl)-7-(2,3-dichlorophenyl)-8-fluoro-2-methylquinoline-3-carboxylate by racemization

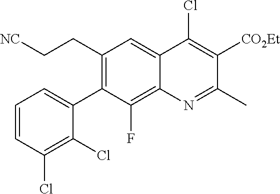 [SMILES: CCOC(=O)c1c(C)nc2c(F)c(-c3cccc(Cl)c3Cl)c(CCC#N)cc2c1Cl]

A mixture of ethyl (Sₐ)-4-chloro-6-(2-cyanoethyl)-7-(2,3-dichlorophenyl)-8-fluoro-2-methylquinoline-3-carboxylate (100 g) in sulfolane (200 mL) was heated to 185° C. for 2 h to give racemic ethyl 4-chloro-6-(2-cyanoethyl)-7-(2,3-dichlorophenyl)-8-fluoro-2-methylquinoline-3-carboxylate. The mixture was cooled to 50° C. and MeCN (200 mL) was added. To the solution was added water (700 mL) at 50° C. The mixture was cooled to r.t. and stirred for 4 h. The solids were isolated by filtration and the wet cake was washed with water (2×200 mL). The wet solids were dried under vacuum at about 50° C. overnight to give the title compound (97 g, 97% yield). LCMS calc. for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

The alternative atropisomer ethyl-(Sₐ)-4-chloro-6-(2-cyanoethyl)-7-(2,3-dichlorophenyl)-8-fluoro-2-methylquinoline-3-carboxylate is convertible to a racemic mixture using an analogous process.

### Step 14. tert-Butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

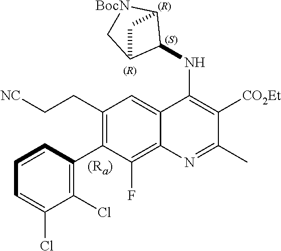

A mixture of ethyl (Rₐ)-4-chloro-6-(2-cyanoethyl)-7-(2,3-dichlorophenyl)-8-fluoro-2-methylquinoline-3-carboxylate (40 g, 85 mmol), Li₂CO₃ (19 g, 258 mmol) and tert-butyl (1R,4R,5S)-5-amino-2-azabicyclo[2.1.1]hexane-2-carboxylate oxalate (29.4 g, 98 mmol) in DMSO (120 mL) was heated to 80° C. overnight. The reaction mixture was cooled to r.t., diluted with MTBE (300 mL) and filtered. The solids were rinsed with MTBE (100 mL). The combined filtrate was washed with water (2×320 mL). The organic phase was separated.

The solvent was removed under reduced pressure to give the title compound that was used for the next step without further purification. Analytical sample was purified by FCC (0-10% MeOH in DCM). LCMS calc. for C₃₂H₃₃Cl₂FN₄O₄: 626.19; Found: 627 (M+H⁺). ¹H-NMR (400 MHz, DMSO-d₆) b 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J=4.1 Hz, 1H), 3.65-3.43 (m, 1H), 3.18 (d, J=9.3 Hz, 1H), 3.02 (s, 1H), 2.91-2.74 (m, 2H), 2.70 (dd, J=13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 6H).

The alternative atropisomer tert-butyl (1R,4R,5S)-5-(((Sₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate is prepared by an analogous route by performing an analogous process starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 14a. tert-Butyl (1R,4R,5S)-5-(((R,)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

The title compound can be alternatively prepared by the following method. A mixture of ethyl (Rₐ)-4-chloro-6-(2-cyanoethyl)-7-(2,3-dichlorophenyl)-8-fluoro-2-methylquinoline-3-carboxylate (2840 g, 5.98 mol) tert-butyl (1R,4R,5S)-5-amino-2-azabicyclo[2.1.1]hexane-2-carboxylate (1449 g, 6.58 mol), LiCl (510 g, 12.0 mol), DIPEA (2331 g, 18 mol) in DMSO (5.7 L) was heated to 80° C. for 24 h. The reaction mixture was cooled to r.t. and MTBE (28 L) and water (14 L) were subsequently added. The organic phase was separated and washed with water (14 L). The solvent was removed under reduced pressure to give the title compound that was used for the next step without further purification. Analytical sample was purified by FCC (0-10% MeOH in DCM). LCMS calc. for C₃₂H₃₃Cl₂FN₄O₄: 626.19; Found: 627 (M+H⁺). ¹H-NMR (400 MHz, DMSO-d₆) b 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J=4.1 Hz, 1H), 3.65-3.43 (m, 1H), 3.18 (d, J=9.3 Hz, 1H), 3.02 (s, 1H), 2.91-2.74 (m, 2H), 2.70 (dd, J=13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 9H).

The alternative atropisomer tert-butyl (1R,4R,5S)-5-(((Sₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate is prepared by an analogous route by performing an analogous process starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 15. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

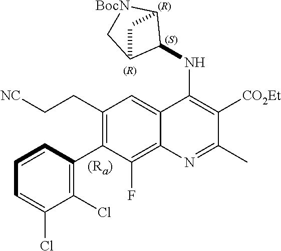

Aq. NaOH (2 M; 134 mL, 268 mmol) was added to a solution of tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (140.0 g, 223 mmol) in MeCN (560 mL) and MeOH (210 mL) at r.t. The mixture was heated to 50° C. for 1-1.5 h. The mixture was cooled to r.t. and acidified with aq. HCl (1 M) to about pH 5. MeCN and MeOH were removed under vacuum. The product was extracted by EtOAc (1.7 L). The aqueous phase was separated and extracted with EtOAc (420 mL). The combined EtOAc phases were concentrated under vacuum to give a residue. MTBE (300 mL) was added to the residue and the mixture slurry was agitated at r.t. for 2 h. The solids were isolated by filtration and the wet cake was washed with MTBE (2×100 mL). The solids were dried under vacuum at about 50° C. to give the title compound (135 g, quantitative) that was used for next step without further purification.

The alternative atropisomer (Sₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid is prepared by an analogous route by performing processes analogous to Steps 14 and 15b starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 15a. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

The title compound can be alternatively prepared by the following process. Sodium trimethylsiloxide (922 g, 95%, 7.81 mol) was added to a solution of tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (3890 g, 6.1 mol) in THF (38.3 L) and water (220 g, 15.62 mol) at r.t. The mixture was heated to 50° C. for 1-3 h to complete the reaction. The mixture was cooled to r.t. and acidified with aq. HCl (1.2 M) to about pH 5. THF was removed under vacuum. The product is extracted by DCM (24 L). The aqueous phase was separated and extracted with DCM (6 L). The combined organic phases were concentrated under vacuum to give the product in DCM solution (12 L). The concentrated DCM solution was added to MTBE (40 L) to precipitate the product while distilling the solvents (total 30 L) under vacuum while n-heptane (20 L) was added to the mixture. The mixture was stirred at 15-30° C. for 1 h. The solids were isolated, and the wet cake was washed with n-heptane (2×3 L). The solids were dried under vacuum at about 50° C. to give the title compound (4020 g) that was used for next step without further purification.

The alternative atropisomer (Sₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid is prepared by an analogous route by performing processes analogous to Steps 14 and 15b starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 16. tert-Butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

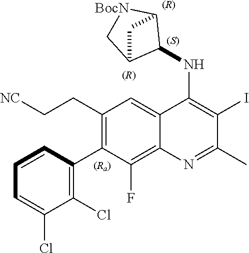

To a mixture of 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (2090 g, 95 wt %, 3.31 mol), and Na₃PO₄ (1131 g, 96%, 6.67 mol) in anhydrous MeCN (25.1 L) at 15-20° C. was added NIS (1412 g, 95%, 5.96 mol) and the mixture was stirred for 1 h. Water (4.0 L) was added to the mixture and resulting slurry was stirred for 1 h at r.t. Water (21.1 L) was added to the mixture gradually and the mixture was agitated for no less than 1 h. The solids were isolated, and the wet cake was reslurried in water (25.1 L) at r.t. for no less than 4 h. The solids were isolated, and the wet cake was washed with water (2×3 L). The solids were dried under vacuum at about 50° C. to give the title compound (2100 g, 97 wt %, 93% overall yield in 3 steps). LCMS calc. for C₃₉H₂₈Cl₂FIN₄O₂: 680.06; Found: 681 (M+H⁺). ¹H-NMR (400 MHz, DMSO-d₆) δ 7.94 (s, 1H), 7.82 (dd, J=8.0, 1.6 Hz, 1H), 7.56 (t, J=7.8 Hz, 1H), 7.50 (dd, J=7.7, 1.6 Hz, 1H), 5.49 (s, 1H), 4.28 (s, 2H), 3.09 (s, 1H), 2.96-2.58 (m, 8H), 1.71 (s, 1H), 1.59-0.96 (m, 11H).

The alternative atropisomer tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate is prepared by an analogous route by performing processes analogous to Steps 14-16 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 17. tert-butyl (1R,4R,5S)-5-(((Rₐ)-6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate

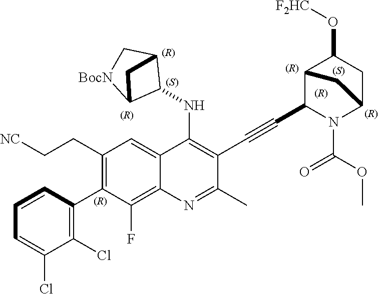

A mixture of methyl (1R,3R,4R,5S)-5-(difluoromethoxy)-3-ethynyl-2-azabicyclo[2.2.1]heptane-2-carboxylate (349 g, 1.42 mol), tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (900 g, 98 wt %, 1.29 mol), CuI (12.33 g, 65 mmol) in DMSO (6.2 L) was degassed and refilled with N₂. To another reactor was added Pd(OAc)₂ (8.72 g, 39 mmol), tri-(4-fluorophenyl)phosphine (49.1 g, 155 mmol) and DMSO (0.45 L). The mixture was degassed and refilled with N₂ and agitated to a solution.

The palladium catalyst solution was added to the reaction mixture in the bigger reactor. The mixture was heated to 50° C. for 1 h. EtOAc (5 L) was added to the reactor, followed by water (10 L). The organic phase was separated, and the aqueous phase was extracted with EtOAc (5 L). The combined organic phase was washed with 5% aq. NH₄OH (3 L). The organic phase was treated twice with 0.5 M N-acetyl cysteine (400 gram) and K₃PO₄ (604 g) in water ((5.7 L) at 50-60° C. for 2 h. The mixture was cooled to 15-20° C. The aqueous phase was separated. The organic phase was washed with water (3 L) and concentrated and azeotroped with toluene (3 L) to give a residual. LCMS calc. for C₄₀H₄₀Cl₂F₂N₅O₅: 798.69, Found: 798 (M+H). ¹H NMR (500 MHz, DMSO) b 8.04 (s, 1H), 7.81 (dd, J=8.1, 1.5 Hz, 1H), 7.55 (t, J=7.9 Hz, 1H), 7.36 (d, J=7.7 Hz, 1H), 6.94-6.59 (m, 2H), 4.58 (d, J=6.9 Hz, 1H), 4.56-4.22 (m, 4H), 3.66 (s, 3H), 3.61-3.48 (m, 1H), 3.19 (t, J=8.2 Hz, 1H), 3.00 (ddd, J=13.6, 6.5, 3.2 Hz, 1H), 2.87 (d, J=10.8 Hz, 1H), 2.83-2.75 (m, 2H), 2.68 (dd, J=14.0, 6.3 Hz, 2H), 2.60 (s, 3H), 2.18-2.02 (m, 2H), 1.86-1.60 (m, 3H), 1.36 (s, 1H), 1.02-0.81 (m, 9H). ¹³C NMR (126 MHz, DMSO) δ 161.78, 154.92, 134.92, 132.79, 131.67, 131.48, 129.22, 125.58, 120.06, 119.86, 119.56, 117.48, 98.19, 97.35, 80.39, 78.32, 75.72, 65.67, 56.60, 56.29, 52.84, 50.78, 50.44, 49.37, 46.08, 41.50, 40.77, 34.01, 33.25, 28.68, 27.73, 25.53, 17.74. ¹⁹F NMR (470 MHz, DMSO) δ −78.98-−81.58 (m), −124.49 (d, J=81.5 Hz).

The alternative atropisomer tert-butyl (1R,4R,5S)-5-(((Sₐ)-6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate is prepared by an analogous route by performing processes analogous to Steps 14-16 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 17a. tert-butyl (1R,4R,5S)-5-(((R,)-6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate

The title compound can alternatively be prepared by the following procedure. A mixture of methyl (1R,3R,4R,5S)-5-(difluoromethoxy)-3-ethynyl-2-azabicyclo[2.2.1]heptane-2-carboxylate (16.7 g, 62.6 mmol), tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (40 g, 56.9 mmol), tris(dibenzylideneacetone)dipalladium(0) (0.52 g, 0.57 mmol), tetrabutylammonium acetate (68.7 g, 228 mmol) in DMF (480 mL) was degassed and refilled with N₂. The mixture was heated to 60° C. for 1-3 h. The mixture was cooled to 15-30° C. and transferred into water (1 L) at r.t. to precipitate the product. The solids were isolated and redissolved in EtOAc (200 mL). The EtOAc solution was treated twice with 0.5 M N-acetyl cysteine and aq. K₃PO₄ (200 mL) at 50-60° C. for no less than 1 h. The mixture was cooled to 15-30° C. and the aqueous phase was separated. The organic solvent was removed by rotavapor under reduced pressure at no more than 40° C. to a residue. The residue was purified by FCC and eluted with 0-30% acetone in heptane to give the title compound (122 g, 90% yield). LCMS calc. for C₄₀H₄₀Cl₂F₂N₅O₅: 798.69, Found: 798 (M+H). ¹H NMR (500 MHz, DMSO) b 8.04 (s, 1H), 7.81 (dd, J=8.1, 1.5 Hz, 1H), 7.55 (t, J=7.9 Hz, 1H), 7.36 (d, J=7.7 Hz, 1H), 6.94-6.59 (m, 2H), 4.58 (d, J=6.9 Hz, 1H), 4.56-4.22 (m, 4H), 3.66 (s, 3H), 3.61-3.48 (m, 1H), 3.19 (t, J=8.2 Hz, 1H), 3.00 (ddd, J=13.6, 6.5, 3.2 Hz, 1H), 2.87 (d, J=10.8 Hz, 1H), 2.83-2.75 (m, 2H), 2.68 (dd, J=14.0, 6.3 Hz, 2H), 2.60 (s, 3H), 2.18-2.02 (m, 2H), 1.86-1.60 (m, 3H), 1.36 (s, 1H), 1.02-0.81 (m, 9H). ¹³C NMR (126 MHz, DMSO) δ 161.78, 154.92, 134.92, 132.79, 131.67, 131.48, 129.22, 125.58, 120.06, 119.86, 119.56, 117.48, 98.19, 97.35, 80.39, 78.32, 75.72, 65.67, 56.60, 56.29, 52.84, 50.78, 50.44, 49.37, 46.08, 41.50, 40.77, 34.01, 33.25, 28.68, 27.73, 25.53, 17.74. ¹⁹F NMR (470 MHz, DMSO) δ −78.98-−81.58 (m), −124.49 (d, J=81.5 Hz).

The alternative atropisomer tert-butyl (1R,4R,5S)-5-(((Sₐ)-6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate is prepared by an analogous route by performing processes analogous to Steps 14-16 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 18. tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate

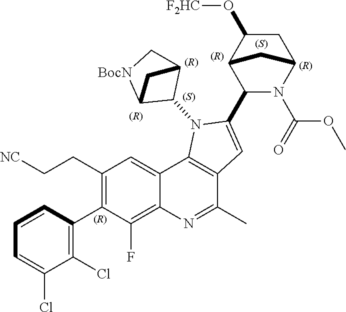

To a solution of tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate (2069 g, 2.84 mol) and Cs₂CO₃ (1110 g, 3.41 mol) in DMSO (8 L) or NMP (8 L) was heated to 80-85° C. for 1-3 h. The reaction is cooled to 15-20° C. and EtOAc (10 L) and water (20 L) were sequentially added while maintain the temperature below 25° C. The organic phase was separated and washed with water (20 L). The combined aqueous phases were extracted with EtOAc (10 L). The combined organic phases were treated with 0.5 M N-acetyl cysteine and K₃PO₄ (604 g) in water 5.7 L) at 50-60° C. for 2 h. The mixture was cooled to 15-30° C. and the aqueous phase was separated. The organic phase was washed with water (10 L), evaporated under vacuum, and dissolved in acetone (12 L). Water (6 L) was added. The mixture was heated to 40° C. to a solution. The seeds of tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate were added. Water (2 L) was added over 30 min. to initiate the crystallization. Water (4 L) was added slowly to the mixture. The mixture was cooled to 15-30° C. and agitated for no less than 1 h. The solids were isolated and washed with water-acetone (1/1, v/v) (2×4 L). The wet cake was dried under vacuum at no more than 55° C. to give the title compound (1857 g, 88% over 2 steps). C₄₀H₄₀Cl₂F₂N₅O₅: 798.69, Found: 798 (M+H). ¹H NMR (600 MHz, DMSO) b 8.16 (d, J=5.1 Hz, 1H), 7.81 (d, J=8.0 Hz, 1H), 7.54 (t, J=7.8 Hz, 1H), 7.38 (t, J=8.8 Hz, 1H), 6.97-6.67 (m, 1H), 6.62 (d, J=10.9 Hz, 1H), 5.29 (t, J=4.6 Hz, 1H), 5.01 (d, J=6.8 Hz, 1H), 4.97-4.88 (m, 1H), 4.68-4.55 (m, 1H), 4.26 (d, J=9.6 Hz, 1H), 3.75 (s, 2H), 3.71-3.53 (m, 3H), 3.21 (dd, J=9.5, 3.4 Hz, 1H), 2.94 (dt, J=14.1, 6.8 Hz, 1H), 2.85 (dt, J=16.5, 6.6 Hz, 1H), 2.82-2.75 (m, 1H), 2.72 (s, 3H), 2.71-2.59 (m, 2H), 2.33-2.15 (m, 2H), 1.75-1.62 (m, 1H), 1.58 (t, J=8.8 Hz, 1H), 1.47 (d, J=13.9 Hz, 2H), 0.59 (d, J=8.5 Hz, 9H). ¹³C NMR (126 MHz, DMSO) δ 156.18, 154.15, 153.76, 153.64, 141.27, 135.59, 135.38, 133.14, 132.76, 131.86, 131.63, 131.27, 129.09, 122.06, 121.24, 120.40, 119.98, 119.51, 118.27, 103.69, 77.92, 75.52, 64.49, 59.23, 57.42, 56.92, 53.00, 49.64, 48.97, 46.28, 42.52, 40.96, 33.74, 31.76, 28.70, 27.60, 22.57, 17.46. ¹⁹F NMR (565 MHz, DMSO) δ −79.43-−81.31 (m), −122.31 (d, J=13.3 Hz).

The alternative atropisomer tert-butyl (1R,4R,5S)-5-((S,)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate is prepared by an analogous route by performing processes analogous to Steps 14-16 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 19. Methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 1)

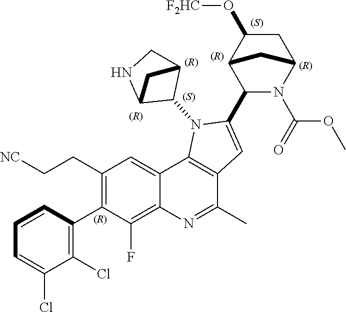

To a solution of tert-butyl (1R,4R,5S)-5-((R,)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate (1350 g, 1.69 mol) in DCM (6.48 L) and 1,1,1,3,3,3-hexfluoroisopropanol (4.3 L) was added 4 M HCl in 1,4-dioxane (2.54 L, 10.1 mol) at no more than 25° C. The mixture was added to 1 M aq. NaOH (7 L) at no more than 20° C. To the mixture was added aq. NaHCO₃ to adjust pH to 7-8. The organic phase was separated and washed with water (4 L). The organic phase was concentrated under vacuum to a residue at no more than 40° C. The residual was dissolved and purified by FCC, eluting with 0-15% of MeOH in EtOAc. The desired fractions were collected and concentrated under reduced pressure to isolate product (1270 g, 77 wt %, 970 g product weight, 82%). The impure fractions were combined and concentrated for further purification. LCMS calc. for C₃₅H₃₂Cl₂F₃N₅O₃: 697.18; Found: 698.10 (M+H⁺). ¹H NMR (500 MHz, DMSO-d₆) b 8.18 (s, 1H), 7.80 (dd, J=7.9, 1.5 Hz, 1H), 7.55 (t, J=7.8 Hz, 1H), 7.44 (s, 1H), 6.88 (d, J=75.8 Hz, 1H), 6.65 (s, 1H), 4.97 (s, 1H), 4.85 (s, 1H), 4.56 (d, J=19.1 Hz, 1H), 4.26 (d, J=13.5 Hz, 2H), 3.92-3.35 (m, 4H), 3.11-2.56 (m, 10H), 2.24 (s, 1H), 1.89 (s, 1H), 1.67 (dd, J=21.0, 13.5 Hz, 1H), 1.50 (d, J=22.4 Hz, 2H), 1.25 (d, J=7.3 Hz, 1H), only peaks visible. ¹⁹F NMR (470 MHz, DMSO) δ-74.58, −122.62.

The alternative atropisomer methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate is prepared by an analogous route by performing processes analogous to Steps 14-18 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Example 2. Synthesis of methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate (Compound 1-hemifumarate) and Compound 1-hemifumarate hemimethanol

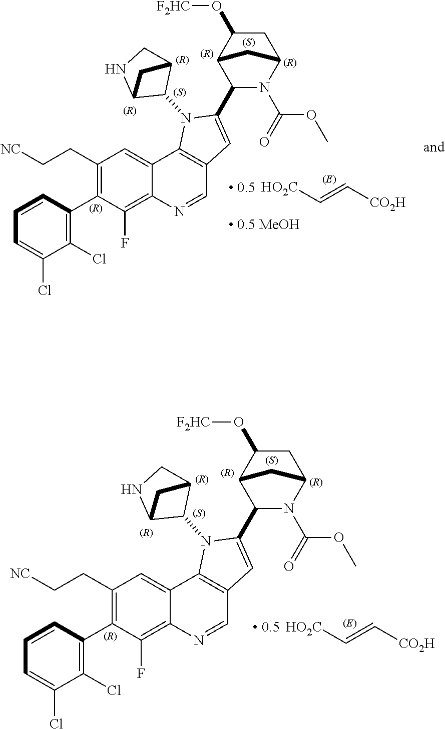

A solution of fumaric acid (78 g, 0.67 mol) in MeOH (800 mL), EtOAc (400 mL) and MTBE (400 mL) was added to a solution of methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (1260 g (970 g), 1.39 mol) in MeOH (400 mL), EtOAc (2000 mL) and MTBE (2000 mL) at 15-30° C. When 25% of the fumaric acid solution was added, the mixture was seeded with methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate seeds. The procedure is optionally performed without adding seed crystals, however, a longer time of agitation may be required to initiate crystallization. The mixture was agitated for no less than 30 min to initiate the crystallization. The rest of the fumaric acid was slowly added at 15-30° C. After the acid addition, MTBE (4000 mL) was added (the final solvent composition by volume is MeOH (1.25 V), EtOAc (2.5 V) and MTBE (6.5 V)). The mixture was agitated at 15-30° C. for no less than 1 h. The solids were isolated, and the wet cake was washed with MTBE (2×2000 mL). If needed, the wet cake could be reslurried in MeOH (970 mL), EtOAc (1940 mL) and MTBE (6790 mL) to produce Compound 1 hemifumarate hemimethanol. The wet cake was dried under vacuum at no more than 65° C. to give final product of Compound 1 hemifumarate (942 g, 90% yield). LCMS calc. for C₃₅H₃₂Cl₂F₃N₅O₃: 698.57; Found: 698 (M+H⁺). ¹H-NMR (500 MHz, DMSO-d₆) b 8.16 (s, 1H); 7.82 (dd, 1H, J=8.1, 1.6 Hz); 7.56, (t, 1H, J=7.8 Hz); 7.43-7.47 (m, 1H); 6.81 (t, 1H, J_{HF}=75.7 Hz); 6.63-6.70 (m, 1H); 6.5 (s, 2H); 5.11-5.21 (m, 1H); 4.86-4.90 (m, 1H); 4.53-4.61 (m, 1H); 4.41-4.45 (d, 1H); 4.24-4.31 (m, 1H); 3.62-3.72 (s, 3H); 3.59-3.65 (m, 1H); 3.13 (m, 1H); 3.06 (m, 1H); 2.97 (m, 1H); 2.72-2.84 (m, 2H); 2.86 (m, 1H); 2.76 (S, 3H); 2.69 (m, 1H); 2.21-2.30 (m, 1H); 1.96-2.03 (m, 1H); 1.63-1.74 (m, 1H); 1.47-1.59 (m, 2H); 1.37-1.42 (m, 1H). ¹³C NMR (126 MHz, DMSO-d6) δ 156, 154.5 (J_{CF}=249.6 Hz); 141.9; 154.4; 141.9; 135.1, 135.1, 131.4; 131.3; 129.1; 120.5; 118.3; 117.5 (¹J_{CF}=263.4 Hz, ²JCF=75.7 Hz); 103.6; 75.5; 62.9; 58.5; 57.1; 56.7; 44.8; 43.0; 40.9; 40.1; 52.9; 49.2; 31.9; 31.4; 28.6; 22.6; 17.4. ¹⁹F NMR (525 MHz, DMSO-d6) δ −122.42 (s, 1H); −79.6-−81.25 (m, 2F).

The alternative atropisomer methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate or hemifumarate hemimethanol is prepared by an analogous route starting from methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate instead of methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate.

Compound 1 hemifumarate salt was confirmed as a crystalline solid according to XRPD analysis. The XRPD pattern is shown in FIG. 1 and the peak data are provided in Table 1. Compound 1 hemifumarate hemimethanol salt was confirmed as a crystalline solid according to XRPD analysis. The calculated XRPD pattern is shown in FIG. 16.

The DSC thermogram is shown in FIG. 2. It dehydrated first below 150° C. and then followed by exothermal event of melting/decomposition at an onset temperature of 234.8° C. and a peak temperature of 237.8° C.

The TGA thermogram is shown in FIG. 3. Weight loss of ˜1.9% was observed below 100° C. due to loss of water. The compound decomposes above 100° C. with weight loss of ˜10.6% up to 300° C.

Recrystallization of Compound 1-hemifumarate

A solution of methyl (1R,3R,4R,5S)-3-((R)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate (100 g) in DCM (600 mL) and MeOH (600 mL) was agitated to a solution. The solution was polished filtered and solvent swapped at below 40° C. to MeOH under vacuum distillation to initiate the crystallization. The MeOH solvent was swapped into EtOH under vacuum distillation at below 40° C. The mixture was cooled to r.t. and agitated for 30 min. The mixture was cooled to about 0° C. for 1 h. The solids were isolated and washed with EtOH and dried under vacuum to give desired product (98 g, 98% yield). LCMS calculated for C₃₅H₃₂Cl₂F₃N₅O₃: 697.18; Found: 698 (M+H⁺). ¹H-NMR (500 MHz, DMSO-d₆) b 8.16 (s, 1H); 7.82 (dd, 1H, J=8.1, 1.6 Hz); 7.56, (t, 1H, J=7.8 Hz); 7.43-7.47 (m, 1H); 6.81 (t, 1H, J_{HF}=75.7 Hz); 6.63-6.70 (m, 1H); 6.5 (s, 2H); 5.11-5.21 (m, 1H); 4.86-4.90 (m, 1H); 4.53-4.61 (m, 1H); 4.41-4.45 (d, 1H); 4.24-4.31 (m, 1H); 3.62-3.72 (s, 3H); 3.59-3.65 (m, 1H); 3.13 (m, 1H); 3.06 (m, 1H); 2.97 (m, 1H); 2.72-2.84 (m, 2H); 2.86 (m, 1H); 2.76 (S, 3H); 2.69 (m, 1H); 2.21-2.30 (m, 1H); 1.96-2.03 (m, 1H); 1.63-1.74 (m, 1H); 1.47-1.59 (m, 2H); 1.37-1.42 (m, 1H). ¹³C NMR (126 MHz, DMSO-d6) δ 156, 154.5 (J_{CF}=249.6 Hz); 141.9; 154.4; 141.9; 135.1, 135.1, 131.4; 131.3; 129.1; 120.5; 118.3; 117.5 (¹J_{CF}=263.4 Hz, ²J_{CF}=75.7 Hz); 103.6; 75.5; 62.9; 58.5; 57.1; 56.7; 44.8; 43.0; 40.9; 40.1; 52.9; 49.2; 31.9; 31.4; 28.6; 22.6; 17.4. ¹⁹F NMR (525 MHz, DMSO-d6) δ −122.42 (s, 1H); −79.6-−81.25 (m, 2F).

### Example 3. Alternative synthesis for preparing ethyl 4-chloro-6-(2-cyanoethyl)-7-(2,3-dichlorophenyl)-8-fluoro-2-methylquinoline-3-carboxylate (step 13 of Example 1

### Step 1. Methyl 6-(2-(1,3-dioxolan-2-yl)ethyl)-3-amino-2′,3′-dichloro-2-fluoro-[1,1′-biphenyl]-4-carboxylate

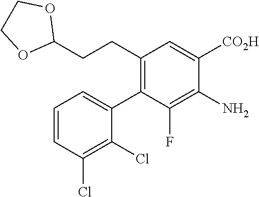 [SMILES: Nc1c(C(=O)O)cc(CCC2OCCO2)c(-c2cccc(Cl)c2Cl)c1F]

To a round bottom flask under N₂ was charged 2-vinyl-1,3-dioxolane (6.52 g, 65.1 mmol.). 0.5 M 9-BBN dimer in THF (149 mL, 74.4 mmol) was cannulated to the reaction flask over 15 min at r.t. The reaction mixture was then heated to 40° C. and stirred for NLT 1 h. After full consumption of the 2-vinyl-1,3-dioxolane, methyl 3-amino-6-bromo-2′,3′-dichloro-2-fluoro-[1,1′-biphenyl]-4-carboxylate (25.0 g, 62.0 mmol) was added, followed by bis(di-tert-butyl(4-dimethylaminophenyl)phosphine)dichloropalladium(II) (0.088 g, 0.124 mmol, 2 mol %), K₃PO₄ (26.3 g, 124 mmol), and water (25 mL). The resulting reaction mixture was sparged by N₃ for another 15 min and heated to 60° C. The reaction was completed after stirring at 60° C. for 16 h. Upon completion, the reaction was quenched by 1 M aq. HCl until pH=7. The reaction mixture was then diluted by EtOAc (50 mL, 2 V) and the organic layer was separated. The aqueous layer was washed by another portion of EtOAc (25 mL, 1V) and the organic layer was separated. The combined organic phase was dried on Na₂SO₄ and filtered through a pad of diatomaceous earth. The filtrate was collected and the solvent was evaporated in vacuo. The crude product was re-dissolved MeCN (150 mL, 6 V) at 60° C. and slowly added water (100 mL, 4 V) to precipitate solid products. The solid slurry was stirred at 60° C. for 2 h then slowly cooled to r.t. to stir for an additional 2 h before isolation. The reaction mixture was filtered and solids were collected. The wet solid cake was washed by 4 V of 30% v/v MeCN in water. After drying the solids by pulling air through for overnight, the title compound was obtained as light grey solids (19.2 g, yield=75%). LCMS calc. for C₁₉H₁₃Cl₂FNO₄: 414.25, Found: 415 (M+H). ¹H NMR (400 MHz, DMSO-d6) δ 7.75 (dd, J=8.1, 1.5 Hz, 1H), 7.54 (d, J=1.4 Hz, 1H), 7.49 (t, J=7.9 Hz, 1H), 7.36 (dd, J=7.7, 1.6 Hz, 1H), 6.48 (s, 2H), 4.60 (t, J=4.8 Hz, 1H), 3.85 (s, 3H), 3.80-3.59 (m, 4H), 2.33 (ddd, J=14.3, 9.4, 6.9 Hz, 1H), 2.22 (ddd, J=14.3, 9.5, 6.8 Hz, 1H), 1.56 (ttd, J=9.6, 7.0, 4.7 Hz, 2H) ppm.

### Step 2. 6-(2-(1,3-Dioxolan-2-yl)ethyl)-3-amino-2′,3′-dichloro-2-fluoro-[1,1′-biphenyl]-4-carboxylic acid

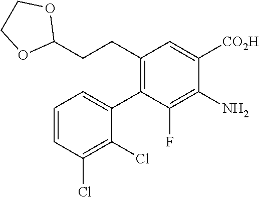 [SMILES: Nc1c(C(=O)O)cc(CCC2OCCO2)c(-c2cccc(Cl)c2Cl)c1F]

Methyl 6-(2-(1,3-dioxolan-2-yl)ethyl)-3-amino-2′,3′-dichloro-2-fluoro-[1,1′-biphenyl]-4-carboxylate (19.2 g, 46.3 mmol) was dissolved in a mixture of THF (80 mL) and MeOH (20 mL). To the reaction mixture was added 1 M aq. NaOH (93 mL) and heated to 50° C. for 3 h. Upon completion, the mixture was cooled to r.t. and slowly acidified by 1 M aq. HCl to pH=3-4. The resulting solid slurry was stirred at r.t. for NLT 1 h. The solid was isolated, and the wet cake was washed by 20% v/v MeCN in water. After drying the solids by pulling air through for overnight, the title compound was obtained as white solids (18.5 g, yield=99%). LCMS calc. for C₁₈H₁₆Cl₂FNO₄:400.23; Found: 401 (M+H). ¹H NMR (400 MHz, DMSO-d₆) δ 7.75 (dd, J=8.0, 1.5 Hz, 1H), 7.53 (d, J=1.4 Hz, 1H), 7.49 (t, J=7.9 Hz, 1H), 7.36 (dd, J=7.7, 1.6 Hz, 1H), 4.60 (t, J=4.8 Hz, 1H), 3.79-3.60 (m, 4H), 3.31 (s, 2H), 3.28 (s, 1H), 2.32 (ddd, J=14.5, 9.4, 6.9 Hz, 1H), 2.21 (ddd, J=14.3, 9.5, 6.8 Hz, 1H), 1.56 (dddd, J=9.4, 7.0, 4.8, 2.4 Hz, 2H) ppm.

### Step 3. Ethyl 6-(2-(1,3-dioxolan-2-yl)ethyl)-7-(2,3-dichlorophenyl)-8-fluoro-2-methyl-4-oxo-1,4-dihydroquinoline-3-carboxylate

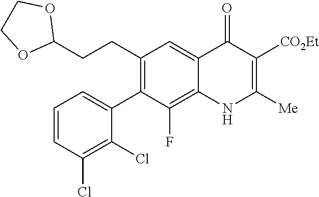 [SMILES: CCOC(=O)c1c(C)[nH]c2c(F)c(-c3cccc(Cl)c3Cl)c(CCC3OCCO3)cc2c1=O]

To a reaction round bottom flask purged by N₂ was dissolved 6-(2-(1,3-dioxolan-2-yl)ethyl)-7-(2,3-dichlorophenyl)-8-fluoro-2H-benzo[d][1,3]oxazine-2,4(1H)-dione (18.6 g, 43.6 mmol) and sodium (Z)-4-ethoxy-4-oxobut-2-en-2-olate (13.28 g, 87.0 mmol) in DMSO (100 mL, 5 V). The mixture was stirred at r.t. for 15 min until fully dissolved. The mixture was then heated to 60-65° C. for 16 h followed by an additional 2 h at 90° C. Upon completion, the mixture was cooled to r.t. and diluted by water (100 mL, 5 V). The resulting reaction mixture was acidified by HCl until pH=7. The solid slurry was reheated to 45-50° C. for 2 h and cooled to r.t. After stirring at r.t. overnight, the crude product was isolated. The crude product was reslurried in 1:1 v/v MTBE in hexane for 2 h at r.t. The solid was then isolated. The wet cake was washed by hexane and dried overnight by pulling air through. The title compound was obtained as a white solid (17.8 g, 83% yield). LCMS calc. for C₂₄H₂₂Cl₂FNO₅: 494.34; Found: 495 (M+H). ¹H NMR (400 MHz, DMSO-d6) δ 11.82 (s, 1H), 7.86 (s, 1H), 7.82 (dd, J=8.0, 1.6 Hz, 1H), 7.56 (t, J=7.8 Hz, 1H), 7.48 (dd, J=7.7, 1.6 Hz, 1H), 4.66 (t, J=4.7 Hz, 1H), 4.27 (q, J=7.1 Hz, 2H), 3.86-3.58 (m, 4H), 2.56 (d, J=7.9 Hz, 1H), 2.44 (s, 4H), 1.74-1.57 (m, 2H), 1.29 (t, J=7.1 Hz, 3H).

### Step 4. Methyl 3-amino-2′,3′-dichloro-2-fluoro-6-(3-oxopropyl)-[1,1′-biphenyl]-4-carboxylate

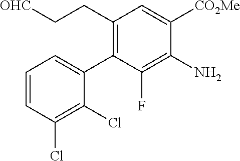 [SMILES: COC(=O)c1cc(CCC=O)c(-c2cccc(Cl)c2Cl)c(F)c1N]

To a solution of methyl 6-(2-(1,3-dioxolan-2-yl)ethyl)-3-amino-2′,3′-dichloro-2-fluoro-[1,1′-biphenyl]-4-carboxylate (6.5 g, 15.7 mmol) in 1,4-dioxane (65 mL) was added 1 M HCl (110 mL). The mixture was heated to 70-75° C. for 2 h. The mixture was cooled to r.t. and MTBE (30 mL) was added. The organic phase was separated, and the aqueous phase was back extracted with MTBE (3×30 mL). The combined organic phase was washed with water (2×30 mL). The organic phase was concentrated to give the title compound (6.14 g, 88%). LCMS calc. for C₁₇H₁₄Cl₂FNO₃: 369.03; Found: 370 (M+H). ¹H NMR (400 MHz, DMSO-d₆) δ 9.54 (d, J=1.1 Hz, 1H), 7.76 (dd, J=8.1, 1.5 Hz, 1H), 7.59-7.45 (m, 2H), 7.39 (dd, J=7.7, 1.5 Hz, 1H), 6.51 (s, 3H), 3.86 (s, 4H), 2.68-2.52 (m, 2H), 2.50-2.34 (m, 3H).

### Step 5. Methyl 3-amino-2′,3′-dichloro-6-(2-cyanoethyl)-2-fluoro-[1,1′-biphenyl]-4-carboxylate

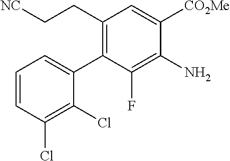 [SMILES: COC(=O)c1cc(CCC#N)c(-c2cccc(Cl)c2Cl)c(F)c1N]

To a mixture of methyl 3-amino-2′,3′-dichloro-2-fluoro-6-(3-oxopropyl)-[1,1′-biphenyl]-4-carboxylate (5.2 g, 11.6 mmol), hydroxylamine hydrochloride (0.9 g, 12.8 mmol) and NEt₃ (1.3 g, 12.8 mmol) in DMF (51 mL) was added propane phosphonic acid anhydride (10.8 mL, 15.3 mmol) and stirred for 3 h. The reaction mixture is diluted with water (100 mL) and extracted with MTBE (3×100 mL). The combined organic phase was washed with water (2×50 mL) and dried over Na₂SO₄. The organic solvent was removed in vacuo and the residue was purified by FCC (0-10% MeOH in DCM) to give the title compound (1.7 g, 40%). LCMS calc. for: C₁₇H₁₃Cl₂FN₂O₂: 366.03; Found: 367 (M+1). ¹H NMR (400 MHz, DMSO-d₆) δ 7.78 (dd, J=8.1, 1.5 Hz, 1H), 7.68 (d, J=1.5 Hz, 1H), 7.52 (t, J=7.9 Hz, 1H), 7.42 (dd, J=7.6, 1.6 Hz, 1H), 6.61 (s, 2H), 3.87 (s, 3H), 2.67-2.51 (m, 2H), 2.47-2.35 (m, 1H).

### Step 6. Ethyl 4-chloro-6-(2-cyanoethyl)-7-(2,3-dichlorophenyl)-8-fluoro-2-methylquinoline-3-carboxylate

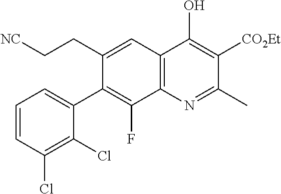 [SMILES: CCOC(=O)c1c(C)nc2c(F)c(-c3cccc(Cl)c3Cl)c(CCC#N)cc2c1O]

A mixture of methyl 3-amino-2′,3′-dichloro-6-(2-cyanoethyl)-2-fluoro-[1,1′-biphenyl]-4-carboxylate (10.00 g, 27.0 mmol) ethyl (Z)-3-ethoxybut-2-enoate (5.3 g, 32.4 mmol) and pyridinium p-toluenesulfonate (0.14 g, 0.54 mmol) in anhydrous toluene (100 mL) was heated at reflux (110° C.) for 21 h. The mixture was cooled to r.t., 2.68 M NaOEt in EtOH (15.1 mL, 40.5 mmol) was then added. The resulting orange solution was heated at 80° C. for 4 h. The reaction mixture was cooled to r.t., diluted with water (100 mL), acidified with 1 M HCl (42 mL) to pH 5. EtOAc (500 mL) was added to the reaction mixture. The organic layer was separated, dried over Na₂SO₄, filtered, and concentrated in vacuo. The residue was purified by FCC eluting with 0-70% EtOAc/DCM to give the title compound as a light-yellow solid (7.2 g, 59% yield). LCMS calc. for C₂₂H₁₇Cl₂FN₂O₂: 446.03; Found: 447 (M+H⁺). ¹H NMR (400 MHz, DMSO-d₆) δ 11.87 (s, 1H), 8.00 (s, 1H), 7.84 (dd, J=7.9, 1.7 Hz, 1H), 7.71-7.48 (m, 2H), 4.28 (q, J=7.1 Hz, 2H), 2.79 (ddd, J=11.7, 7.4, 3.7 Hz, 1H), 2.73-2.59 (m, 3H), 2.46 (s, 3H), 1.30 (t, J=7.1 Hz, 3H).

### Example 4. Synthesis of methyl (1R,3R,4R,5S)-5-(difluoromethoxy)-3-ethynyl-2-azabicyclo[2.2.1]heptane-2-carboxylate (step 17 in Example 1)

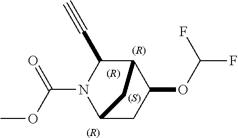

### Step 1. Methyl (1R,3R,4R,5S)-5-hydroxy-2-((S)-1-phenylethyl)-2-azabicyclo[2.2.1]heptane-3-carboxylate

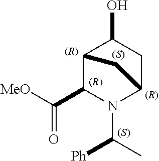

9-BBN (0.5 M in THF, 4.57 L, 2.283 mol) was slowly added to methyl (1R,3R,4S)-2-((S)-1-phenylethyl)-2-azabicyclo[2.2.1]hept-5-ene-3-carboxylate (470 g, 1.826 mol) in anhydrous THF (2.35 L), while maintaining the temperature at 5° C. during addition. The mixture was warmed to r.t. and stirred for 16 h. Water (9.8 mL) was slowly added into the reaction to quench the excess 9-BBN. An ice-cold premixed solution of 32% aq. H₂O₂ (874 mL, 9.132 mol) and 2M NaOH (3.20 L) was slowly added to the reaction mixture over 9 h at 0° C. as the process was exothermic. The reaction temperature was kept around 3° C. during addition. The reaction mixture was warmed to r.t. and stirred for 1 h. The reaction mixture was diluted with EtOAc (4.7 L), the layers were separated, and the aqueous layer was extracted with EtOAc (4.7 L). The combined organic layer was washed with brine (4.7 L) and concentrated under reduced pressure. The residue was purified over FCC eluding with a gradient of 0 to 70% EtOAc in heptanes to give the title compound (240.0 g, 48% yield) as yellow solid. LCMS calc. for C₁₆H₂₁NO₃: 275.4, found 276 (M+1). ¹H NMR (400 MHz, CDCl3) δ 7.51-7.09 (m, 5H), 4.07-3.90 (m, 1H), 3.99-3.70 (m, 2H), 3.38 (s, 1H), 3.29 (s, 3H), 2.46 (ddd, J=15.1, 7.5, 3.0 Hz, 2H), 2.16-2.01 (m, 1H), 1.77 (d, J=10.1 Hz, 1H), 1.37 (t, J=13.7 Hz, 4H).

### Step 2. Methyl (1R,3R,4R,5S)-5-hydroxy-2-((S)-1-phenylethyl)-2-azabicyclo[2.2.1]heptane-3-carboxylate

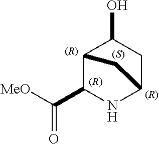

Methyl (1R,3R,4R,5S)-5-hydroxy-2-((S)-1-phenylethyl)-2-azabicyclo[2.2.1]heptane-3-carboxylate (201 g, 730 mmol) and 20% Pd(OH)₂ (10.25 g, 50% wet) in MeOH (2 L) was charged into two Parr 2 L shaker bottles. The mixture was hydrogenated at 50 psi overnight. The mixture was filtered through a diatomaceous earth pad, which was rinsed with MeOH (1 L). The solvent was removed under reduced pressure to give crude product, which was used subsequently. LCMS calc. for C₈H₁₃NO₃: 171.2, found 172 (M+1). ¹H NMR (400 MHz, DMSO-d6) b 4.10 (m, 1H), 3.95 (m, 2H), 3.79 (s, 3H), 2.65 (s, 1H), 2.09 (dd, 1H), 1.90 (d, 1H), 1.57 (d, 1H), 1.39 (d, 1H).

### Step 3. Methyl (1R,3R,4R,5S)-5-hydroxy-2-azabicyclo[2.2.1]heptane-3-carboxylate

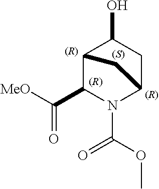

Methyl chloroformate (59.1 mL, 766.5 mmol) was slowly added to a solution of crude methyl (1R,3R,4R,5S)-5-hydroxy-2-((S)-1-phenylethyl)-2-azabicyclo[2.2.1]heptane-3-carboxylate (calc. weight 125 g, 730 mmol) and NEt₃ (122 mL) in DCM (1 L) at 0° C. (exothermic), while maintaining the temperature <25° C. during addition. After stirring at r.t. overnight, the mixture was cooled to 5° C., then sequentially diluted with 1 M HCl (146 mL) and water (600 mL). The layers were separated, and the aqueous layer was extracted with DCM (600 mL). The combined organic layers were washed with water (600 mL). The organic layer was concentrated under reduced pressure to give crude product (176.0 g) as a yellow oil which was used subsequently. LCMS calc. for C₁₀H₁₅NO₅: 229.3, found 230.2 (M+1). ¹H NMR (400 MHz, CDCl₃) δ 4.25 (m, 1H), 4.05 (m, 1H), 3.79 (s, 3H), 3.50-3.80 (m, 4H), 2.55-2.55 (m, 2H), 2.03 (br m, 1H), 1.70-1.80 (m, 2H), 1.39 (d, 1H).

### Step 4. Dimethyl (1R,3R,4R,5S)-5-hydroxy-2-azabicyclo[2.2.1]heptane-2,3-dicarboxylate

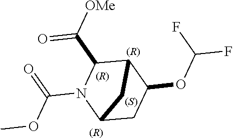

Crude methyl (1R,3R,4R,5S)-5-hydroxy-2-azabicyclo[2.2.1]heptane-3-carboxylate (183.4 g, 800 mmol) in DCM (660 mL) was added to a solution of potassium bifluoride (468.6 g, 6 mol) in water (660 m) in a 4 L plastic bottle at 0° C. To the above reaction mixture (bromodifluoromethyl)-trimethylsilane (622.1 mL, 4 mol) was slowly added over 3 h. The reaction temperature was kept at 12 to 18° C. during addition. After stirring for 1 h, water (500 mL) was added to the reaction mixture. The layers were separated, and the aqueous layer was extracted with DCM (2×300 mL). The combined organic layers were washed with water (500 mL). The DCM solution was passed through a silica gel plug (70 g), which was rinsed with 400 mL of 40% EtOAc in heptanes (400 mL). The filtrate was concentrated under reduced pressure to give the title compound (178.0 g, 637.4 mmol, 80%, over three steps) as yellow oil. LCMS calc. for C₁₁H₁₅F₂NO₅: 279.33, found: 280 (M+1). ¹H NMR (400 MHz, CDCl3) δ 6.63-5.89 (m, 1H), 4.42 (d, J=8.7 Hz, 1H), 4.00-3.44 (m, 7H), 2.86 (s, 1H), 2.48-2.12 (m, 1H), 2.12-2.01 (m, 1H), 2.01-1.86 (m, 1H), 1.83-1.59 (m, 2H). ¹⁹F NMR (376 MHz, CDCl₃) δ −81.91 (d, J=4.9 Hz), −81.96-−82.16 (m), −82.22 (d, J=15.4 Hz).

### Step 5. Methyl (1R,3R,4R,5S)-5-(difluoromethoxy)-3-(hydroxymethyl)-2-azabicyclo[2.2.1]heptane-2-carboxylate

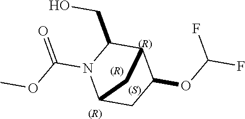

In a jacketed reactor, 2 M lithium borohydride in THF (743 mL, 1.486 mol) was slowly added to a solution of dimethyl (1R,3R,4R,5S)-5-hydroxy-2-azabicyclo[2.2.1]heptane-2,3-dicarboxylate (166 g, 594.5 mmol) in THF (660 mL) at 0° C. The temperature was kept 5-10° C. during addition. The mixture was slowly warmed to r.t. and stirred for 3 h. The mixture was cooled to −5° C. with the jacketed reactor set at −15° C. Sat. NH₄Cl (300 ml) was slowly added dropwise to the reaction mixture (very exothermic). The mixture was diluted with water (300 mL) and EtOAc (900 mL). The layers were separated, and the aqueous layer was extracted with EtOAc (900 mL). The combined organic layers were dried over with Na₂SO₄ (200 g) and concentrated under reduced pressure. This material purified on a Biotage automated chromatography system (800 g column), eluding with a gradient of 15 to 100% EtOAc in heptanes to give the title compound (132 g 79% combined yield) as a white solid. LCMS calc. for C₁₀H₁₅F₂NO₄: 251.10, Found: 252.2 (M+1). ¹HNMR (400 MHz, CDCl₃) δ 6.22 (t, 1H), 4.37 (d, 1H), 4.21 (s, 1H), 3.94 (d, 1H), 3.75-3.60 (m, 5H), 3.38 (m, 1H), 2.56 (s, 1H), 2.12 (dd, 1H), 1.80-1.65 (m, 3H). ¹⁹F NMR (376 MHz, CDCl₃) δ −81.46-−81.72 (m), −81.77, −81.82-82.50 (m).

### Step 6. Methyl (1R,3R,4R,5S)-5-(difluoromethoxy)-3-formyl-2-azabicyclo[2.2.1]heptane-2-carboxylate

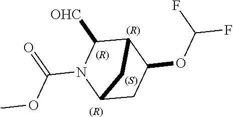

Methyl (1R,3R,4R,5S)-5-(difluoromethoxy)-3-(hydroxymethyl)-2-azabicyclo[2.2.1]heptane-2-carboxylate (20 g, 80 mmol) was dissolved in DCM (300 mL) followed by addition of Na₂CO₃ (4.22 g, 39.8 mmol) and NaHCO₃ (33.4 g, 398 mmol) at r.t. Then the reactor was put in an ice bath at 15° C. with addition of water (300 mL) to cool to 2° C. To the mixture was added NaBr (0.819 g, 7.96 mmol) and TEMPO (0.622 g, 3.98 mmol). With vigorous stirring, trichloroisocyanuric acid (8.33 g, 35.8 mmol) in 5 portions was slowly added during about 15 min to control the temperature between 0.5 to 2° C. After the reaction was completed, the solid was filtered and DCM layer was separated from aqueous phase. The aqueous layer extracted with DCM (150 mL×2) and combined organic layer was dried with Na₂SO₄. The filtered solution was concentrated to obtain a crude oil 18 g aldehyde used for next step. LCMS calc. for C₁₀H₁₃F₂NO₄: 249.10, Found: 250.2 (M+1). ¹H NMR (400 MHz, CDCl₃) δ 9.58 (d, J=17.6 Hz, 1H), 6.35-5.90 (t, 1H), 4.43 (d, 2H), 3.73-3.30 (m, 4H), 2.96 (d, J=5.5 Hz, 1H), 2.21-2.05 (m, 1H), 1.59-1.41 (m, 3H). ¹⁹F NMR (376 MHz, CDCl₃) δ −81.59-−81.88 (m), −81.90-82.29 (m).

### Step 7. Methyl (1R,3R,4R,5S)-5-(difluoromethoxy)-3-ethynyl-2-azabicyclo[2.2.1]heptane-2-carboxylate

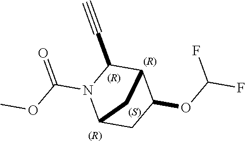

To tert-butyl (1R,3R,4R,5S)-5-(difluoromethoxy)-3-formyl-2-azabicyclo[2.2.1]heptane-2-carboxylate (46 g, 158 mmol) was added MeOH (395 mL) at r.t. followed by addition of K₂CO₃ (87 g, 632 mmol). Bestmann reagent diethyl (1-diazo-2-oxopropyl)phosphonate (40 g) was added slowly. After half of the reagent was added, the reaction was slightly exothermic from 22° C. to 24° C. The reaction mixture was then put in a water/ice bath to keep the temperature around 22° C. The mixture was stirred overnight. Solid salt was filtered off and the filtrate was concentrated to a thick syrup. Water (300 mL) was added to dissolve the oil and white precipitation formed while oil dissolved. MTBE (250 mL) was added to the aqueous mixture to extract the solid followed by MTBE (100 mL) extraction. MTBE was dried with Na₂SO₄ followed by concentration to obtain an oil, which was purified by FCC using 0 to 20% EtOAc in heptane as eluent. 25 g product obtained. LCMS calc. for C₁₁H₁₃F₂NO₃: 245.2, found 246.1 (M+1). ¹H NMR (400 MHz, CDCl₃) δ 6.62-5.91 (m, 1H), 4.47-4.22 (m, 2H), 4.01-3.83 (m, 1H), 3.73 (s, 3H), 2.81 (s, 1H), 2.34 (d, J=8.5 Hz, 1H), 2.28-1.99 (m, 2H), 1.96-1.77 (m, 1H), 1.77-1.54 (m, 1H). ¹³C NMR (101 MHz, CDCl₃) δ 155.00, 154.83, 118.33, 115.73, 113.14, 81.69, 81.34, 77.23, 73.89, 73.84, 71.30, 71.09, 56.23, 55.97, 52.59, 50.38, 49.94, 49.72, 49.60, 41.30, 40.70, 33.54, 32.75. ¹⁹F NMR (376 MHz, CDCl₃) δ −81.92-82.33 (m).

### Example 5. Alternative synthesis of methyl (1R,3R,4R,5S)-5-(difluoromethoxy)-3-ethynyl-2-azabicyclo[2.2.1]heptane-2-carboxylate (step 17 in Example 1)

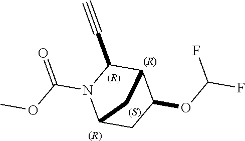

### Step 1. (1R,4S)-2-benzyl-2-azabicyclo[2.2.1]hept-5-en-3-one

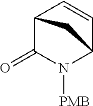

Into a 20 L reactor was added BnBr (1.6 kg, 9.35 mol), DMF (5 L) and (1R,4S)-2-azabicyclo[2.2.1]hept-5-en-3-one (1 kg, 9.16 mol). The reaction mixture was stirred under N₂ and cooled to 0° C. Into the reaction mixture was added NaH (60% in mineral oil, 385 g, 9.62 mol) portion-wise over about 1 h while the internal temperature was maintained below 35° C. The reaction mixture was stirred at r.t. for 2 h and then slowly added into 0.1 N HCl (5 L). The mixture was extracted with MTBE (5×5 L). The combined organic layer was washed with semi-brine (2×5 L) and dried under vacuum. The crude material was further dried by azeotrope with toluene (2×4L×2) to afford dry crude product. The crude product was then dissolved in MeCN (5 L) and washed with heptane (2 L) to remove mineral oil. The MeCN layer was concentrated under reduced pressure to afford the crude product (1.64 kg, 80 wt % purity, 72% yield) as brown oil, which was used in next step without further purification. ¹HNMR (400 MHz, CDCl₃) δ 7.36-7.27 (m, 3H), 7.22-7.20 (m, 2H), 6.57 (s, 2H), 4.47 (d, J=14.8 Hz, 1H), 4.05 (s, 1H), 3.98 (d, J=15.2, 1H), 3.40 (s, 1H), 2.32 (d, J=7.6 Hz, 1H), 2.10 (d, J=7.6 Hz, 1H). ¹³C NMR (101 MHz, CDCl₃) δ 180.0, 139.5, 137.3, 136.5, 128.5, 128.3, 127.5, 62.6, 58.3, 53.7, 48.0. LCMS calc. for C₁₃H₁₃NO (M+H)+: 200.1, found 200.1.

### Step 2. (1R,4S,5S)-2-benzyl-5-hydroxy-2-azabicyclo[2.2.1]heptan-3-one

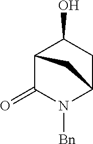 [SMILES: O=C1[C@H]2C[C@H](C[C@@H]2O)N1Cc1ccccc1]

Into a dry 20 L reactor was added (1R,4S)-2-benzyl-2-azabicyclo[2.2.1]hept-5-en-3-one (1.31 kg, 80.00 wt % purity, 5.26 mol) and dry THF (6.5 L). The reaction mixture was cooled to 3° C. BH₃·DMS (460 g, 6.05 mol) was added to the reaction mixture dropwise over about 1 h, while the internal temperature of the reaction mixture was maintained below 30° C. The reaction mixture was stirred at r.t. for 2 h, then the reaction mixture was cooled to −10° C., transferred slowly to 0° C. water (4 L). Sodium perborate monohydrate (0.92 kg, 9.21 mol) was slowly charged at r.t. to the mixture over 4 portions. 10 N NaOH (526 mL) was charged to the reaction mixture and the mixture was stirred overnight. The reaction mixture was diluted with EtOAc (3.5 L) and layers were separated. The organic layer was collected, filtered through diatomaceous earth bed, and concentrated under reduced pressure to afford the crude product. Into the crude product was added MTBE (13 L) and the mixture was heated at 50° C. for no less than 2 h. The mixture was decanted, and the clear organic solution was collected. The organic solution was concentrated under reduced pressure to about 5 L, during which a precipitate of undesired regioisomer was formed. The slurry was then filtered, and the filter cake was washed with MTBE (1.3 L). The combined organic solutions were concentrated further under reduced pressure to afford light brown thick paste enriched with the title compound (37%). ¹HNMR (400 MHz, CDCl₃) δ 7.37-7.23 (m, 5H), 4.64 (d, J=15.2 Hz, 1H), 4.31 (d, J=6.8 Hz, 1H), 3.93-3.88 (m, 2H), 3.71 (s, 1H), 2.92 (s, 1H), 2.06-1.94 (m, 3H), 1.55 (d, J=13.2 Hz, 1H). ¹³C NMR (101 MHz, CDCl₃) δ 176.3, 136.5, 128.7, 128.0, 127.7, 70.3, 58.6, 54.8, 44.8, 38.7, 37.1. LCMS calc. for C₁₃H₁₅NO₂ (M+H)+: 218.1, found 218.1.

### Step 3. (1R,4S,5S)-2-benzyl-5-(difluoromethoxy)-2-azabicyclo[2.2.1]heptan-3-one

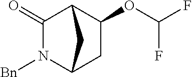 [SMILES: O=C1[C@H]2C[C@H](C[C@@H]2OC(F)F)N1Cc1ccccc1]

Into a dry 20 L reactor was added (1R,4S,5S)-2-benzyl-5-hydroxy-2-azabicyclo[2.2.1]heptan-3-one (1.1 kg, 3.84 mol, 75.5 wt % purity of both regioisomers), MeCN (6.6 L) and CuI (146 g, 0.77 mol). The reaction mixture was heated to 45° C. A solution of 2-(fluorosulfonyl)difluoroacetic acid (0.6 L, 5.78 mol) in MeCN (2.2 L) was added into the reaction mixture over 2 h via dropping funnel while the internal temperature was maintained between 45-55° C. Then the reaction mixture was stirred at 50° C. for 1 h. The reaction mixture was cooled to 0° C. and quenched by slow addition of sat. Na₂CO₃ (6.6 L). The reaction mixture was diluted with MTBE (4.4 L) and stirred for 20 min. The organic layer was decanted, filtered through diatomaceous earth bed and concentrated under vacuum. The crude product was in MTBE (8.8 L) and filtered through diatomaceous earth bed. The organic solution was collected and concentrated to afford crude product as a yellow oil. Purification of the crude product by FCC using heptane and EtOAc gave the tile compound (400 g) as oil, which solidified upon standing. ¹HNMR (400 MHz, CDCl₃) δ 7.38-7.24 (m, 5H), 6.22 (t, J=74.4 Hz, 1H), 4.63 (d, J=15.0 Hz, 1H), 4.58 (d, J=7.0 Hz, 1H), 3.95 (d, J=15.0 Hz, 1H), 3.74 (s, 1H), 3.08 (s, 1H), 2.12-2.00 (m, 2H), 1.92 (d, J=9.7 Hz, 1H), 1.74 (d, J=13.7 Hz, 1H). ¹³C NMR (101 MHz, CDCl₃) δ 174.1, 136.4, 128.8, 128.0, 127.8, 116.0 (t, J=260.5 Hz), 72.9 (t, J=4.5 Hz), 58.0, 52.7, 44.9, 37.8, 37.6. LCMS calc. for C₁₄H₁₅F₂NO₂ (M+H)+: 268.1, found 268.1.

### Step 4. (1R,3R,4R,5S)-2-benzyl-5-(difluoromethoxy)-3-((trimethylsilyl)ethynyl)-2-azabicyclo[2.2.1]heptane

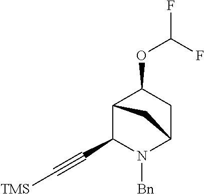 [SMILES: C[Si](C)(C)C#C[C@H]1[C@H]2C[C@H](C[C@@H]2OC(F)F)N1Cc1ccccc1]

Into a 20 L reactor was added (1R,4S,5S)-2-benzyl-5-(difluoromethoxy)-2-azabicyclo[2.2.1]heptan-3-one (810 g, 96 wt % purity, 2.9 mol) and toluene (6.5 L). Vaska's complex (1.13 g, 1.45 mmol, 0.05 mol %) was added at r.t. The resulting yellow solution was sparged with dry N₂ for 10 min. Then 1,1,3,3-tetramethyldisiloxane (429 g, 3.19 mol) was added into the reaction mixture over 10 min while maintaining the internal temperature between 17-23° C. Gas evolved over the course of silane addition and the yellow color of the reaction solution diminished overtime. After silane addition, the reaction mixture was stirred at r.t. for 10 min. Trimethylsilylacetylene (428 g, 4.36 mol) was added into the reaction mixture followed by CuBr (20.8 g, 145 mmol, 5 mol %). The reaction turned turbid after CuBr addition. The slurry was stirred at r.t. for 16 h and then filtered through diatomaceous earth bed to get crude solution. The crude solution was dried under vacuum to afford 1.09 kg crude product (77 wt % purity, assay yield 83%) used in next step without further purification. ¹HNMR (400 MHz, CDCl₃) δ 7.39-7.26 (m, 5H), 6.22 (t, J=74.8 Hz, 1H), 4.24 (s, 1H), 3.69-3.62 (m, 2H), 3.31 (s, 1H), 2.70 (s, 1H), 2.63 (s, 1H), 2.45-2.39 (m, 1H), 2.01 (d, J=10.1 Hz, 1H), 1.68 (d, J=10.1 Hz, 1H), 1.46 (d, J=14.5 Hz, 1H), 0.12 (s, 9H). ¹³C NMR (101 MHz, CDCl₃) δ 138.8, 129.0, 128.1, 127.0, 115.95 (t, J=259.8 Hz), 106.2, 86.9, 75.0, 58.8, 55.7, 55.6, 50.6, 33.5, 33.4, 0.0. LCMS Calcuated for C₁₉H₂₅F₂NOSi (M+H)⁺: 350.1, found 350.2.

### Step 5. (1R,3R,4R,5S)-2-benzyl-5-(difluoromethoxy)-3-ethynyl-2-azabicyclo[2.2.1]heptane

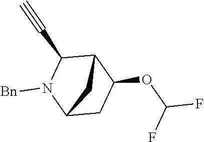 [SMILES: C#C[C@H]1[C@H]2C[C@H](C[C@@H]2OC(F)F)N1Cc1ccccc1]

To a solution of (1R,3R,4R,5S)-2-benzyl-5-(difluoromethoxy)-3-((trimethylsilyl)ethynyl)-2-azabicyclo[2.2.1]heptane (1.62 kg, 77 wt % purity, 3.56 mol) in MeOH (8.1 L) was slowly added K₂CO₃ (615 g, 4.45 mol) at r.t. The reaction mixture was stirred at r.t. for 2 h. The crude mixture was diluted with MTBE (13 L) and filtered through diatomaceous earth bed. The crude solution was then concentrated under vacuum to afford the crude product, which was purified by FCC using heptane and EtOAc to afford the title compound (957 g, 97% yield). ¹HNMR (400 MHz, CDCl₃) δ 7.39-7.25 (m, 5H), 6.22 (t, J=74.4 Hz, 1H), 4.27 (s, 1H), 3.77 (d, J=13.2 Hz, 1H), 3.57 (d, J=13.2 Hz, 1H), 3.29 (s, 1H), 2.71 (s, 1H), 2.65 (s, 1H), 2.44 (brs, 1H), 2.24 (s, 1H), 2.02 (d, J=10.0 Hz, 1H), 1.71 (d, J=10.2 Hz, 1H), 1.46 (d, J=14.4 Hz, 1H). ¹³C NMR (101 MHz, CDCl₃) δ 138.7, 128.9, 128.2, 127.1, 115.92 (t, J=260.0 Hz), 84.3, 74.8, 70.6, 58.2, 55.1, 54.9, 50.6, 33.4, 33.2. LCMS calc. for C₁₆H₁₇F₂NO: 278.1, found 278.1.

### Step 6. Methyl (1R,3R,4R,5S)-5-(difluoromethoxy)-3-ethynyl-2-azabicyclo[2.2.1]heptane-2-carboxylate

(1R,3R,4R,5S)-2-benzyl-5-(difluoromethoxy)-3-ethynyl-2-azabicyclo[2.2.1]heptane (54.43 g, 185 mmol) was dissolved in DCM (27 mL) and methyl chloroformate (86 mL, 1112 mmol) was added followed by K₂HPO₄ (3.23 g, 18.53 mmol). The mixture was stirred at 40° C. for 20 h. The mixture was diluted with MTBE (380 mL) and transferred into a solution of K₃PO₄ (118.27 g, 555 mmol) in water (260 mL). The organic layer was collected, and the solvent was evaporated under reduced pressure to provide a light brown foam. FCC using EtOAc-heptane (1:3) afforded product (40 g, 77% IY, >99% GCAP) as an off-white solid. ¹HNMR (400 MHz, CDCl₃) δ 6.21 (t, J=74.0 Hz, 1H), 4.43-4.24 (m, 2H), 4.02-3.82 (m, 1H), 3.73 (s, 3H), 2.81 (s, 1H), 2.40-2.03 (m, 3H), 1.82 (d, J=8.1 Hz, 1H), 1.69 (d, J=14.3 Hz, 1H). ¹³C NMR (101 MHz, DMS-d₆) δ 154.7, 117.4 (t, J=170.7 Hz), 82.6, 75.5, 74.1, 56.5, 52.7, 49.6, 49.2, 40.6, 33.5. LCMS calc. for C₁₁H₁₃F₂NO₃ (M+H)⁺: 246.0, found 246.1.

### Example 6. Alternative synthesis of methyl (1R,3R,4R,5S)-5-(difluoromethoxy)-3-ethynyl-2-azabicyclo[2.2.1]heptane-2-carboxylate (step 17 in Example 1)

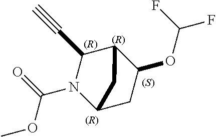

### Step 1. (1R,4S)-2-(4-methoxybenzyl)-2-azabicyclo[2.2.1]hept-5-en-3-one

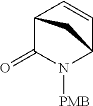

(1R,4S)-2-azabicyclo[2.2.1]hept-5-en-3-one (10 g, 92 mmol) was dissolved anhydrous THF (100 mL) and 4-methoxybenzyl chloride (14.85 mL, 110 mmol) was added. The suspension was cooled to 10° C. and KOtBu (12.34 g, 110 mmol) was added in portions while maintaining <20° C. After 4 h, the mixture was cooled to 10° C. and diluted with MTBE (50 mL), followed by sat. aq. NaHCO₃ (100 mL). The organic layer was collected, and the solvent was evaporated under reduced pressure to provide a brown oil. FCC using EtOAc-heptane (2:3) afforded product (13.7 g, 65% IY, >98% LCAP) as a yellow oil. ¹HNMR (400 MHz, CDCl₃) δ 7.10 (d, J=8.3 Hz, 2H), 6.84 (d, J=8.6 Hz, 2H), 6.51 (s, 1H), 4.33 (d, J=14.6 Hz, 1H), 4.01-3.96 (m, 2H), 3.79 (s, 2H), 3.36 (s, 1H), 2.26 (d, J=7.6 Hz, 1H), 2.04 (d, J=7.2 Hz, 1H). ¹³C NMR (101 MHz, CDCl₃) δ 179.8, 159.0, 139.6, 137.0, 129.7, 128.4, 113.9, 62.5, 58.3, 55.2, 53.8, 47.3. LCMS calc. for C₁₄H₁₅NO₂ (M+H)⁺: 230.1, found 230.1.

### Step 2. (1R,4S,5S)-5-hydroxy-2-(4-methoxybenzyl)-2-azabicyclo[2.2.1]heptan-3-one

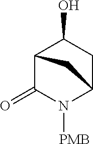

(1R,4S)-2-(4-methoxybenzyl)-2-azabicyclo[2.2.1]hept-5-en-3-one (5 g, 21.81 mmol) was dissolved in anhydrous THF (25 mL) and allylpalladium chloride dimer (0.319 g, 0.872 mmol) was added followed by (R)-(2′-methoxy-[1,1′-binaphthalen]-2-yl)diphenylphosphane (0.817 g, 1.745 mmol). The solution was sparged with N₂ for 10 min and cooled to 10° C. Trichlorosilane (2.86 mL, 28.3 mmol) was added via syringe dropwise. The mixture was stirred at 10-15° C. for 2 h and cooled to 0° C. A mixture EtOH (25 mL) and NEt₃ (9.42 mL) was added dropwise while maintaining <20° C. The resultant slurry was stirred at r.t. for 1 h then filtered and washed with 20 mL of EtOAc. The solvent of the filtrate was removed, and the crude oil was redissolved in MeOH and EtOAc (25 mL/25 mL). To this mixture was added KF (3.93 g, 67.6 mmol) and urea hydrogen peroxide (14.36 g, 153 mmol) at r.t. The slurry was stirred for 2 h and filtered. The filtrate was concentrated to dryness and partitioned between EtOAc and water (50 mL/50 mL). The solvent of the organic layer was removed and the product was stirred in MTBE and heptanes (25 mL/25 mL) for 12 h. The solid of the resultant slurry was isolated via filtration to afford product (3.5 g, 53% IY, 86% LCAP). ¹HNMR (400 MHz, CDCl₃) δ 7.14 (d, J=8.1 Hz, 2H), 6.85 (d, J=8.1 Hz, 2H), 4.52 (d, J=14.8 Hz, 1H), 4.25 (d, J=6.8 Hz, 1H), 4.19 (s, 1H), 3.86 (d, J=14.8 Hz, 1H), 3.79 (s, 3H), 3.67 (s, 1H), 2.88 (s, 1H), 2.00-1.88 (m, 3H), 1.51 (d, J=13.1 Hz, 1H). ¹³C NMR (101 MHz, CDCl₃)b 176.2, 159.1, 129.3, 128.5, 114.1, 70.2, 58.5, 55.3, 54.91, 44.3, 38.7, 37.2. LCMS calc. for C₁₄H₁₇NO₃ (M+H)⁺: 248.1, found 248.2.

### Step 3. (1R,4S,5S)-5-(difluoromethoxy)-2-(4-methoxybenzyl)-2-azabicyclo[2.2.1]heptan-3-one

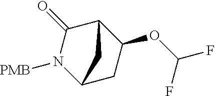

(1R,4S,5S)-5-hydroxy-2-(4-methoxybenzyl)-2-azabicyclo[2.2.1]heptan-3-one (3.5 g, 11.61 mmol) was suspended in anhydrous MeCN, (25 mL) and CuI (0.332 g, 1.741 mmol) was added. The mixture was heated to 50° C. 2-(fluorosulfonyl)difluoroacetic acid (1.679 mL, 16.25 mmol) was dissolved in anhydrous MeCN (7 mL) and added to mixture at 50° C. via syringe while maintaining <60° C. After 2 h, the mixture was cooled to 10° C. and MTBE (15 mL) was added, followed by sat. aq. Na₂CO₃ (21 mL). The organic layer was collected, and the solvent was evaporated under reduced pressure to provide a brown foam. FCC using EtOAc-heptane (1:3) afforded product (2.3 g, 67% IY, >98% LCAP) as a yellow crystalline solid. ¹HNMR (400 MHz, CDCl₃) δ 7.15 (d, J=8.5 Hz, 2H), 6.86 (d, J=8.6 Hz, 2H), 6.19 (t, J=74.0 Hz, 1H), 4.54-4.48 (m, 2H), 3.92 (d, J=14.8 Hz, 1H), 3.80 (s, 3H), 3.70 (s, 1H), 3.04 (s, 1H), 2.07-2.01 (m, 1H), 1.97 (d, J=9.7 Hz, 1H), 1.88 (d, J=9.7 Hz, 1H), 1.70 (d, J=13.6 Hz, 1H). ¹³C NMR (101 MHz, CDCl₃)b 173.9, 159.2, 129.4, 128.4, 115.9 (t, J=261.5 Hz), 114.2, 72.97 (t, J=4.4 Hz), 57.9, 55.3, 52.8, 44.3, 37.81 (d, J=15.4 Hz). LCMS calc. for C₁₅H₁₇F₂NO₃ (M+H)⁺: 298.1, found 298.1.

### Step 4. (1R,3R,4R,5S)-5-(difluoromethoxy)-2-(4-methoxybenzyl)-3-((trimethylsilyl)ethynyl)-2-azabicyclo[2.2.1]heptane

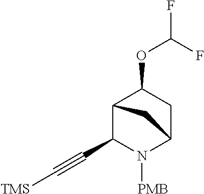

(1R,4S,5S)-5-(difluoromethoxy)-2-(4-methoxybenzyl)-2-azabicyclo[2.2.1]heptan-3-one (4.2 g, 14.13 mmol) was dissolved in anhydrous toluene (40 mL) and chlorocarbonylbis(triphenylphosphine)iridium (1) (11 mg, 0.014 mmol) was added. The solution was sparged with N₂ for 10 min and 1,1,3,3-tetramethyldisiloxane (2.75 mL, 15.54 mmol) was added while maintaining <30° C. The solution was stirred for 20 min before adding trimethylsilylacetylene (3.57 mL, 25.4 mmol) followed by CuBr (101 mg, 0.70 mmol). The mixture was stirred at r.t. for 24 h and quenched via the addition of sat. aq. NaHCO₃ (40 mL). The organic layer was collected, and the solvent was evaporated under reduced pressure to provide a brown oil which was filtered through silica plug to afford 4.8 g of product.

### Step 5. (1R,3R,4R,5S)-5-(difluoromethoxy)-3-ethynyl-2-(4-methoxybenzyl)-2-azabicyclo[2.2.1]heptane

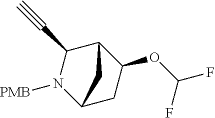

(1R,3R,4R,5S)-5-(difluoromethoxy)-2-(4-methoxybenzyl)-3-((trimethylsilyl)ethynyl)-2-azabicyclo[2.2.1]heptane (4.7 g, 12.38 mmol) was dissolved in MeOH (25 mL), and K₂CO₃ (2.05 g, 14.86 mmol) was added. The slurry was stirred for 2 h and filtered to remove salts. The solvent of the filtrate was evaporated to an oil and purified via FCC using EtOAc-heptane (1:3) to afford the title compound (3.4 g, 77% over 2 steps, >97% GCAP) as a light-yellow oil. ¹HNMR (400 MHz, CDCl₃) δ 7.30-7.27 (m, 2H), 6.87 (d, J=8.1 Hz, 2H), 6.22 (t, J=74.4 Hz, 1H), 4.25 (s, 1H), 3.82 (s, 3H), 3.70 (d, J=13.0 Hz, 1H), 3.50 (d, J=13.0 Hz, 1H), 3.27 (s, 1H), 2.66 (d, J=14.2 Hz, 2H), 2.42 (d, J=10.5 Hz, 1H), 2.24 (s, 1H), 2.00 (d, J=10.0 Hz, 1H), 1.69 (d, J=10.1 Hz, 1H), 1.44 (d, J=14.4 Hz, 1H). ¹³C NMR (101 MHz, CDCl₃) δ 158.7, 130.7, 130.0, 115.9 (t, J=260.5 Hz), 113.6, 84.4, 74.9, 70.6, 58.1, 55.2, 54.7, 54.5, 50.5, 33.3, 33.1. LCMS calc. for C₁₇H₁₉F₂NO₂ (M+H)⁺: 308.1, found 308.2.

### Step 6. (1R,3R,4R,5S)-5-(difluoromethoxy)-3-ethynyl-2-azabicyclo[2.2.1]heptane-2-carboxylate

The title compound was prepared in the same manner as step 6 in Example 5. Example 6a. Alternative synthesis of methyl (1R,3R,4R,5S)-5-(difluoromethoxy)-3-ethynyl-2-azabicyclo[2.2.1]heptane-2-carboxylate (step 17 in Example 1)

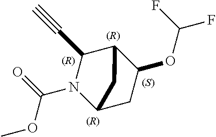

### Step 1. (1R,4S)-2-(4-methoxybenzyl)-2-azabicyclo[2.2.1]hept-5-en-3-one

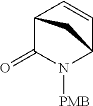

Into the a reactor was charged (1R,4S)-2-azabicyclo[2.2.1]hept-5-en-3-one (50 kg, 458 mol), DMF (150 L) and 4-methoxybenzylchloride (71.8 kg, 458 mol). The reaction mixture was cooled to 10° C. and then KOtBu (56.5 kg, 504 mol) was added to the reactor. The reaction mixture was stirred at 10° C. until completion. It was filtered and the filter cake was rinsed with MTBE (150 L). The filtrate was washed with 30% NH₄Cl aq. (200 L). The aq. layer was extracted with MTBE (150 L) twice. The combined organic layers were washed with 15% aq. NH₄Cl (125 L) twice and then with 10% aq. NaCl (135 L). The organic layer was filtered through silica gel (25 kg). The filter cake was rinsed with MTBE (150 L) and concentrated to dryness. The crude product was dried by azeotroping with toluene (50 L) to afford a light yellow oil (97.7 kg, 93% yield), which was used directly in the next step without further purification. ¹HNMR (400 MHz, CDCl₃) δ 7.10 (d, J=8.3 Hz, 2H), 6.84 (d, J=8.6 Hz, 2H), 6.51 (s, 1H), 4.33 (d, J=14.6 Hz, 1H), 4.01-3.96 (m, 2H), 3.79 (s, 2H), 3.36 (s, 1H), 2.26 (d, J=7.6 Hz, 1H), 2.04 (d, J=7.2 Hz, 1H). ¹³CNMR (101 MHz, CDCl₃) δ 179.8, 159.0, 139.6, 137.0, 129.7, 128.4, 113.9, 62.5, 58.3, 55.2, 53.8, 47.3. LCMS calc. for C₁₄H₁₅NO₂ (M+H)⁺: 230.1, found 230.1.

### Step 2. (1R,4S,5S)-5-hydroxy-2-(4-methoxybenzyl)-2-azabicyclo[2.2.1]heptan-3-one

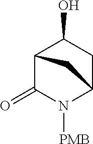

Into a reactor was charged (1R,4S)-2-(4-methoxybenzyl)-2-azabicyclo[2.2.1]hept-5-en-3-one (97 kg, 422 mol), 2-MeTHF (485 L), (R)-MOP (1.98 kg, 4.2 mol) and (Pd-allyl-CI)₂ (0.39 kg, 1.1 mol). Then the reactor was purged with N₂ three times. The reaction mixture was cooled to 10° C. and then trichlorosilane (85.9 kg, 633 mol) was added to the reactor. The reaction mixture was heated to 45° C. and agitated for 2 h and then cooled to 5° C. A mixed solution of NEt₃ (214 kg, 2110 mol) and EtOH (485 L) was added to the reactor. Then the mixture was warmed to 25° C. and agitated for 2 h. The reaction mixture was then filtered and the filter cake was washed with 2-MeTHF (485 L). The filtrate was concentrated to 350 L and the mixture was filtered again. To the filtrate was charged MeOH (485 L), 2-MeTHF (650 L) and KF (73.5 kg, 1266 mol). The mixture was cooled to 15° C. and to the mixture was added urea hydrogen peroxide (79.5 kg, 844 mol). The mixture was then stirred at 20° C. for 2 h before quenching with 10% aq. Na₂SO₃ (1167 L). Then 12% aq. NaOH (100 L) was subsequently added slowly to the reactor. The mixture was agitated for 5 min and the phases were separated. The aqueous phase was extracted with 2-MeTHF (291 L). To the combined organic layer was added 1M aq. HCl until pH=7. The organic layer was concentrated to 150 L under vacuum and then iPrOAc (252 L) was added to the reactor. The mixture was stirred at 45° C. for 30 min, then cooled to 15° C. and stirred for 1 h. The mixture was filtered and the filter cake was rinsed with cold IPAc (97 L). The wet cake was dried under vacuum to obtain a white solid (67.06 kg, 64% yield). ¹HNMR (400 MHz, CDCl₃) δ 7.14 (d, J=8.1 Hz, 2H), 6.85 (d, J=8.1 Hz, 2H), 4.52 (d, J=14.8 Hz, 1H), 4.25 (d, J=6.8 Hz, 1H), 4.19 (s, 1H), 3.86 (d, J=14.8 Hz, 1H), 3.79 (s, 3H), 3.67 (s, 1H), 2.88 (s, 1H), 2.00-1.88 (m, 3H), 1.51 (d, J=13.1 Hz, 1H). ¹³CNMR (101 MHz, CDCl₃) δ 176.2, 159.1, 129.3, 128.5, 114.1, 70.2, 58.5, 55.3, 54.91, 44.3, 38.7, 37.2. LCMS calc. for C₁₄H₁₇NO₃ (M+H)⁺: 248.1, found 248.2.

### Step 3. (1R,4S,5S)-5-(difluoromethoxy)-2-(4-methoxybenzyl)-2-azabicyclo[2.2.1]heptan-3-one

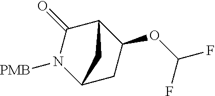

(1R,4S,5S)-5-hydroxy-2-(4-methoxybenzyl)-2-azabicyclo[2.2.1]heptan-3-one (100 g, 394 mmol), CuI (3.75 g, 19.71 mmol) were suspended in MeCN (600 mL). The mixture was heated to 50° C. Then 2,2-difluoro-2-(fluorosulfonyl)acetic acid (61.1 mL, 591 mmol) dissolved in MeCN (200 mL) was added dropwise to the previous mixture at 50° C. Upon completion, the reaction mixture was reverse quenched into cold aq. ammonia (400 mL). The resultant aqueous layer was discarded. The organic layer was concentrated to 150 mL and diluted with MTBE (500 mL). It was then washed with 300 mL of a mixture of 6M aq. ammonia and 10% aq. NH₄Cl solution, followed by water (100 mL) wash. The organic layer was treated with charcoal (10 g) and filtered through diatomaceous earth. The filter cake was washed with MTBE (100 mL). The organic layer was then concentrated to dryness. The crude product was redissolved in EtOH (300 mL) and then water (150 mL) was added, followed by seeding. After 20 min, more water (300 mL) was added. The slurry was stirred for another 3 h, filtered and washed with 200 mL of 2:3 EtOH/water mixture to afford a white solid (89 g, 75% yield). ¹HNMR (400 MHz, CDCl₃) δ 7.15 (d, J=8.5 Hz, 2H), 6.86 (d, J=8.6 Hz, 2H), 6.19 (t, J=74.0 Hz, 1H), 4.54-4.48 (m, 2H), 3.92 (d, J=14.8 Hz, 1H), 3.80 (s, 3H), 3.70 (s, 1H), 3.04 (s, 1H), 2.07-2.01 (m, 1H), 1.97 (d, J=9.7 Hz, 1H), 1.88 (d, J=9.7 Hz, 1H), 1.70 (d, J=13.6 Hz, 1H). ¹³CNMR (101 MHz, CDCl₃) δ 173.9, 159.2, 129.4, 128.4, 115.9 (t, J=261.5 Hz), 114.2, 72.97 (t, J=4.4 Hz), 57.9, 55.3, 52.8, 44.3, 37.81 (d, J=15.4 Hz). LCMS calc. for C₁₅H₁₇F₂NO₃ (M+H)⁺: 298.1, found 298.1.

### Step 4. (1R,3R,4R,5S)-5-(difluoromethoxy)-2-(4-methoxybenzyl)-3-((trimethylsilyl)ethynyl)-2-azabicyclo[2.2.1]heptane

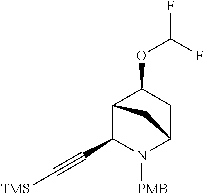

(1R,4S,5S)-5-(difluoromethoxy)-2-(4-methoxybenzyl)-2-azabicyclo[2.2.1]heptan-3-30 one (80 g, 266 mmol) was dissolved in toluene (640 mL). Into the reaction mixture was added chlorocarbonylbis(triphenylphosphine)iridium (1) (0.042 g, 0.053 mmol) at 10° C. The resultant yellow solution was purged with N₂ three times. 1,1,3,3-Tetramethyldisiloxane (51.7 mL, 293 mmol) was added slowly into the reaction mixture. The reaction was stirred at 20° C. for 30 min. Trimethylsilylacetylene (56 mL, 399 mmol) was added into the reaction mixture followed by CuBr (5.72 g, 39.9 mmol). The reaction mixture was stirred at 20° C. for 6 h and then filtered through diatomaceous earth. The filter cake was washed with toluene (80 mL). The combined organic phase was concentrated to dryness to obtain 89 g of an oil. It was used in next step without further purification.

### Step 5. (1R,3R,4R,5S)-5-(difluoromethoxy)-3-ethynyl-2-(4-methoxybenzyl)-2-azabicyclo[2.2.1]heptane

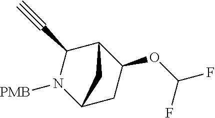

Into crude (1R,3R,4R,5S)-5-(difluoromethoxy)-2-(4-methoxybenzyl)-3-((trimethylsilyl)ethynyl)-2-azabicyclo[2.2.1]heptane (89 g, 235 mmol) was added MeOH (180 mL) and K₂CO₃ (6.48 g). The reaction mixture was stirred at 20° C. for 5 h and diluted with MTBE (450 mL) and 10% aq. NH₄Cl (270 mL). The mixture was stirred for 10 min and then the aq. layer was discarded. The organic layer was washed with 10% aq. NH₄Cl (270 mL). Into the organic layer was added 0.5M HCl (518 mL). The mixture was stirred for 10 min and then organic layer was discarded. The aq. layer was washed with MTBE (450 mL) and then basified with 10 M NaOH to pH=˜12 and extracted with MTBE (540 mL). The organic layer was aged with charcoal (9 g) for 12 h. The reaction mixture was filtered through diatomaceous earth and washed with MTBE (180 mL). The organic solution was concentrated to dryness to afford a tan-colored oil (67.7 g, 91% yield over 2 steps). The product was used in next step without further purification. ¹HNMR (400 MHz, CDCl₃) δ 7.30-7.27 (m, 2H), 6.87 (d, J=8.1 Hz, 2H), 6.22 (t, J=74.4 Hz, 1H), 4.25 (s, 1H), 3.82 (s, 3H), 3.70 (d, J=13.0 Hz, 1H), 3.50 (d, J=13.0 Hz, 1H), 3.27 (s, 1H), 2.66 (d, J=14.2 Hz, 2H), 2.42 (d, J=10.5 Hz, 1H), 2.24 (s, 1H), 2.00 (d, J=10.0 Hz, 1H), 1.69 (d, J=10.1 Hz, 1H), 1.44 (d, J=14.4 Hz, 1H). ¹³CNMR (101 MHz, CDCl₃) δ 158.7, 130.7, 130.0, 115.9 (t, J=260.5 Hz), 113.6, 84.4, 74.9, 70.6, 58.1, 55.2, 54.7, 54.5, 50.5, 33.3, 33.1. LCMS calc. for C₁₇H₁₉F₂NO₂ (M+H)⁺: 308.1, found 308.2.

### Step 6. (1R,3R,4R,5S)-5-(difluoromethoxy)-3-ethynyl-2-azabicyclo[2.2.1]heptane-2-carboxylate

(1R,3R,4R,5S)-5-(difluoromethoxy)-3-ethynyl-2-(4-methoxybenzyl)-2-azabicyclo[2.2.1]heptane (63.71 g, 198 mmol) was dissolved in DCM (30 mL). Methyl chloroformate (107 mL, 1.39 mol) and K₂HPO₄ (3.45 g, 19.82 mmol) were added into the reaction. The mixture was agitated at 40° C. for 17 h. Upon completion, the reaction was cooled to 15° C., diluted with MTBE (300 mL) and NEt₃ (2.76 mL). Then MTBE (320 mL) and DABCO (33.4 g, 297 mmol) were added. The resultant slurry was stirred for 3 h at 25° C. Then water (250 mL) was added to the slurry. The aq. layer was discharged and the organic layer was washed with brine (240 mL). The resultant organic layer was treated with charcoal (6.3 g) for 3 h. The mixture was filtered through diatomaceous earth and washed with MTBE (65 mL). The solvent of the organic solution was distilled down to 120 mL and heptane (63 mL) was added. The resultant mixture was heated to 50° C. for full dissolution and cooled to 40° C. for seeding. The mixture was stirred for 3 h at 40° C., cooled to 20° C. over 12 h. Then heptane (125 mL) was added and the mixture was cooled to 5° C. The product was isolated via filtration to obtain a white solid (42 g, 87% yield) in >99% GC and chiral purity.

### Example 6b. Alternative synthesis of methyl (1R,3R,4R,5S)-5-(difluoromethoxy)-3-ethynyl-2-azabicyclo[2.2.1]heptane-2-carboxylate (step 17 in Example 1)

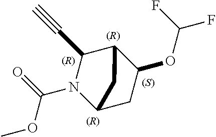

### Step 1. (1R,3R,4R,5S)-3-ethynyl-2-(4-methoxybenzyl)-2-azabicyclo[2.2.1]heptan-5-ol

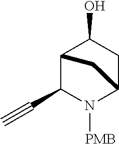

(1R,4S,5S)-5-hydroxy-2-(4-methoxybenzyl)-2-azabicyclo[2.2.1]heptan-3-one (20 kg, 80.9 mol) was dissolved in THF (100 L) and the reaction was purged with N₂ three times. Then chlorocarbonylbis(triphenylphosphine)iridium (1) (8.6 g, 11.0 mmol) and 1,1,3,3-tetramethyldisiloxane (22.8 kg, 169.7 mol) were charged to the reaction. The mixture was agitated under N₂ for 1 h at 25° C. Upon consumption of the starting material, CuBr (1.74 kg, 12.1 mol) and trimethylsilylacetylene (15.9 kg, 161.8 mol) were added. The reaction was agitated for 10 h at 25° C. and quenched with a solution of Na₂SO₃ (5.0 kg, 39.7 mol) in water (100 L). The mixture was diluted with MTBE (80 L) and the aq. layer was discharged. The organic layer was washed three times with 9% aq. ammonia (60 L) and concentrated to an oil. It was then redissolved in MeOH (100 L) and K₂CO₃ (22.4 kg, 162.0 mol) was added. The slurry was agitated at 25° C. for 3 h, filtered through diatomaceous earth and washed with MeOH (40 L). The filtrate was concentrated to dryness and redissolved in 2Me-THF (100 L). A solution of 3M aq. HCl (64 L) was added to the mixture and agitated. The aq. layer was discharged and washed twice with 2-MeTHF (60 L). Then 2-MeTHF (100 L) was added and the biphasic mixture was basified with 30% aq. NaOH until pH=12. Upon phase split, the aq. layer was discharged, the organic layer was washed with 9% aq. NaCl (6.6 L) and concentrated to dryness. The resultant oil was dissolved in a mixture of EtOAc (20 L) and heptane (22 L). It was then filtered through silica gel (20 kg) and the filter cake was rinsed with a mixture of 1:1 EtOAc/heptane (24 L). The filtrate was concentrated (16.53 kg, 79% yield) and solvent-swapped to 60 L toluene for usage in the next step. ¹HNMR (400 MHz, CDCl₃) 7.29 (d, J=8.0 Hz, 2H), 6.86 (d, J=8.4 Hz, 2H), 3.91 (d, J=6.8 Hz, 1H), 3.82 (s, 3H), 3.67 (d, J=12.9 Hz, 1H), 3.49 (d, J=12.9 Hz, 1H), 3.25 (s, 1H), 2.61 (s, 1H), 2.45-2.34 (m, 2H), 2.22 (s, 1H), 1.93 (d, J=10.1 Hz, 1H), 1.85 (brs, 1H), 1.72 (d, J=10.0 Hz, 1H), 1.20 (d, J=14.1 Hz, 1H). ¹³CNMR (101 MHz, CDCl₃) δ 158.6, 131.0, 130.1, 113.5, 85.1, 72.6, 70.2, 58.41, 55.2, 55.0, 54.5, 52.6, 35.1, 33.0. LCMS Calculated for C₁₆H₁₉NO₂ (M+H)⁺: 258.1, found 258.2.

### Step 2. (1R,3R,4R,5S)-3-ethynyl-2-azabicyclo[2.2.1]heptan-5-ol hydrochloride

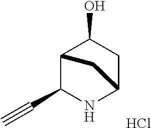

Into a reactor containing a toluene solution of (1R,3R,4R,5S)-3-ethynyl-2-(4-methoxybenzyl)-2-azabicyclo[2.2.1]heptan-5-ol (36.7 kg, 142.6 mol) was charged with DIPEA (3.7 kg, 28.6 mol) and toluene (73 L). The reactor was purged with N₂ followed by addition of 1-chloroethyl chloroformate (40.8 kg, 285.4 mol) at 25° C. The reaction mixture was heated for 2 h at 35° C. Upon completion of the reaction, MeOH (66 L) was added into the reaction mixture, and then the reaction mixture was stirred at 65° C. for 2 h. Then the reaction mixture was concentrated to dryness. The crude product was redissolved in 1M aq. HCl (147 L) and washed with MTBE (184 L). The biphasic mixture was polish-filtered through diatomaceous earth and rinsed with 1M aq. HCl (37 L). The filtrate was phase split, and the aq. layer was washed again with MTBE (110 L). The resultant aq. layer was used in the next step without further purification and isolation.

### Step 3. Methyl (1R,3R,4R,5S)-3-ethynyl-5-hydroxy-2-azabicyclo[2.2.1]heptane-2-carboxylate

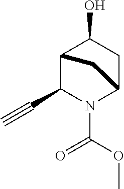

A reactor containing an aq. solution of (1R,3R,4R,5S)-3-ethynyl-2-azabicyclo[2.2.1]heptan-5-ol HCl (184 L, 142.6 mol) was charged with MeCN (49 L) and cooled to 15° C. To this mixture, 30% aq. NaOH (40 kg, 298.2 mol) was added to ensure pH=7-8. Then Na₂CO₃ (30 kg, 284 mol) was added to the reactor, followed by addition of methyl chloroformate (14.7 kg, 155.6 mol). The reaction was stirred at 15° C. for 2 h. Upon consumption of starting material, the mixture was polish-filtered, and the filter cake was rinsed with DCM (73 L). The filtrate was phase separated, and the aq. layer was washed twice with DCM (73 L). The combined organic layers were concentrated to dryness and redissolved in 2-MeTHF (122 L). The solution was treated with charcoal (12.3 kg) at 55° C. for 2 h. It was then filtered through diatomaceous earth, and the filter cake was rinsed with 2-MeTHF (49 L). The filtrate was solvent swapped to IPAc (25 L) at 55° C. followed by gradual cooling to 10° C. The mixture was precipitated during cooling and into the slurry was charged heptane (74 L) and agitated for 1 h. The mixture was filtered, and the filter cake was rinsed with a 1:2 mixture of IPAc/heptane (18 L) to afford the product (17.5 kg, 64% yield over two steps). ¹HNMR (400 MHz, CDCl₃) δ 4.27-4.20 (m, 1H), 4.01 (d, J=5.7 Hz, 1H), 3.87-3.79 (m, 1H), 3.71 (s, 3H), 2.59 (s, 1H), 2.35-2.28 (m, 1H), 2.23 (s, 1H), 2.19-1.95 (m, 2H), 1.86 (m, 1H), 1.47 (dd, J=13.6, 2.8 Hz, 1H). ¹³CNMR (101 MHz, CDCl₃) δ 155.2, 81.6, 71.7, 70.9, 56.6, 52.5, 51.4, 50.0, 42.9, 33.0. LCMS Calculated for C₁₀H₁₃NO₃ (M+H)⁺: 196.0, found 196.1.

### Step 4. (1R,3R,4R,5S)-5-(difluoromethoxy)-3-ethynyl-2-azabicyclo[2.2.1]heptane-2-carboxylate

To a reactor was added KHF₂ (18.6 kg, 238 mol) and water (29 L). To this aq. solution was added methyl (1R,3R,4R,5S)-3-ethynyl-5-hydroxy-2-azabicyclo[2.2.1]heptane-2-carboxylate (5.8 kg, 29.7 mol) and DCM (29 L). The mixture was agitated at 15° C. and (bromodifluoromethyl)trimethylsilane (24.1 kg, 118.7 mol) was slowly charged to the reactor. The resultant mixture was agitated for 1 h. Upon completion of the reaction, the organic layer was separated, and the aq. layer was washed with DCM (17.4 L). The combined organic layer was washed with water (11 L). The organic layer was concentrated to dryness and redissolved in MeOH (11.6 L). To this solution was added 28 wt % aq. ammonia (17.4 kg). The slurry was agitated at 15° C. for 1 h, cooled to 5° C. and agitated for 3 h. The solid was isolated via filtration and rinsed with water (5.8 L). The solid was then redissolved in MeOH (11.6 L) at 45° C. The temperature was lowered to 10° C. and agitated until precipitation. Then water (17.4 L) was charged, and the slurry was agitated for 2 h. The solid was isolated via filtration and rinsed with water (5.8 L) to afford the product (5.6 kg, 77% yield) in >99% GC and chiral purity.

### Example 7. Synthesis of tert-Butyl (1R,4R,5S)-5-amino-2-azabicyclo[2.1.1]hexane-2-carboxylate oxalate (step 14a in Example 1)

### Step 1. (E)-4-Methoxybut-3-en-2-one

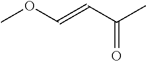

A mixture of 4,4-dimethoxy-2-butanone (350 g, 1.0 eq.) and NaOAc (11 g, 0.05 eq.) was heated to 145-150° C. under N₂ and the resulting MeOH was purged during the heating process. When the reaction was complete, the mixture was cooled to 70-80° C. The product was distilled under vacuum to give the title compound (130 g, 50% yield). ¹H NMR (CD2Cl₂/CD30D, 400 MHz): δ 7.60 (d, 1H, J=12.8 Hz) 5.53 (d, 1H, J=12.8 Hz), 3.81 (s, 3H), 2.17 (s, 3H). ¹³C NMR (CD2Cl₂/CD30D, 100.6 MHz): b 27.1, 58.0, 107.0, 165.2, 199.6.

### Step 2. (E)-4-(Allylamino)but-3-en-2-one

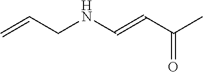

A mixture of (E)-4-methoxybut-3-en-2-one (150 g) and NEt₃ (182 g) in DCM (450 mL) was agitated under N₂ at 10-15° C. Aq. allylamine hydrochloride (60%, 234 g) was slowly added to the mixture at 10-15° C. After the addition, the mixture was agitated for 30 min. When the reaction was completed, water (150 g) was added to the reaction mixture. The organic phase was separated, and the water phase was extracted with DCM (300 mL). The combined organic phases were washed with brine (150 mL) and the organic phase was concentrated under vacuum to give crude product as yellow oil (175 g, yield 93%). ¹H NMR (500 MHz, CDCl₃): δ 9.75 (bs, 1H); 6.58 (dd, 1H, J=16.8, 2); 5.78-5.86 (m, 1H); 5.19 (d, 1H, J=16.8)); 5.14 (d, 1H, J=10, 1)); 5.00 (d, 1H, J=10, 1); 3.74-3.77 (m, 2H); 2.03, (s, 3H). ¹³C NMR (125 Hz, CDCl₃): 197.5; 153.2; 165.3; 117.6; 94.9; 51.1; 29.2.

### Step 3. tert-Butyl (E)-allyl(3-oxobut-1-en-1-yl)carbamate

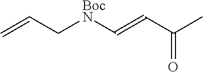 [SMILES: C=CCN(/C=C/C(C)=O)C(=O)OC(C)(C)C]

A mixture of (E)-4-(allylamino)but-3-en-2-one (130 g), trimethylamine (105 g), N,N-dimethylaminopyridine (13 g) in toluene (390 mL) was heated to 50-55° C. (Boc)₂O (259 g) was added in portions while maintaining the reaction temperature between 50-55° C. After the reaction mixture was agitated for 2 h at 50-55° C., the mixture was cooled to 10-15° C. and 3 M aq. HCl was added to the mixture until the pH 5-6. The organic phase was separated, and the aqueous phase was extracted with toluene (260 mL). The combined organic phases were washed with water (260 mL). Activated charcoal (1 g) was added. The mixture was agitated at 50-55° C. for 1 h before cooling the mixture to 20-30° C. The mixture was filtered over diatomaceous earth bed and the diatomaceous earth bed was rinsed with toluene. The filtrate was concentrated to a residue and the residue was co-evaporated with MeCN to give a residue as yellow oil (189 g, 80% yield). ¹H NMR (500 MHz, CDCl₃): δ 8.11 (d, 1H, J=15); 5.68-5.73 (m, 1H); 5.49 (d, 1H, J=15); 5.14 (d, 1H, J=18); 5.09 (d, 1H, J=10); 4.13 (t, 2H); 2.20 (s, 3H); 1.50 (s, 9H). ¹³C NMR (125 MHz, CDCl₃): 198.6; 153.0; 143.2; 131.8; 117.8; 109.5; 84.0; 47.0; 28.3; 28.1.

### Step 4. tert-Butyl 5-acetyl-2-azabicyclo[2.1.1]hexane-2-carboxylate

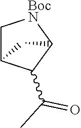

A solution of tert-butyl (E)-allyl(3-oxobut-1-en-1-yl)carbamate (270 g) in MeCN (3240 mL) was subjected to UV-photo reactor. When the reaction was complete, the yellow oil residue (major and minor isomer mixture) was used for next step without further purification. Sample was purified by column to get analytical data. ¹H NMR (500 MHz, CDCl₃) δ 4.62-6.78 (bd, 1H); 3.40 (bt, 1H); 3.16 (bs, 1H); 3.06 (bs, 1H); 2.69 (s, 1H); 1.97 (s, 3H); 1.70-1.73 (m, 1H); 1.46 (s, 9H).

### Step 5. tert-Butyl (1R,4R,5S)-5-amino-2-azabicyclo[2.1.1]hexane-2-carboxylate oxalate

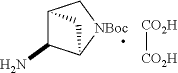 [SMILES: CC(C)(C)OC(=O)N1C[C@H]2C[C@@H]1[C@H]2N ; O=C(O)C(=O)O]

A mixture of tert-butyl 5-acetyl-2-azabicyclo[2.1.1]hexane-2-carboxylate (150 g) in MeCN (1500 mL) was added to sodium hypochlorite (173.5 g) in 30% aq. NaOH (1500 mL) at 30-40° C. The mixture was agitated at 30-40° C. for 30 min. The mixture was cooled to 10-15° C. and 6M HCl was added to adjust the mixture pH 8-9. The mixture was concentrated under vacuum to remove MeCN at 50-55° C. and MeOH (90 mL) was added to the residue. The mixture was cooled to 10-15° C. and 6M HCl was added to adjust the mixture pH 2-3 (solids precipitated out as the pH adjustment) and agitated for additional 2-3 h. The solids were isolated and rinsed with water (300 mL). The wet solids were dried under vacuum at 50-55° C.

Recrystallization: A mixture of the solids in toluene (1500 mL) was heated to 60-70° C. to a solution. (R)-(+)-1-phenylethylamine (80.7 g) was added at 40-70° C. The solution was cooled to 30-35° C. over 90 min and agitated for 1 h. The suspension was cooled to 20-25° C. over 90 min and agitated for 2 h. The solids were isolated and rinsed with toluene (40 mL). A mixture of the cake and toluene (1200 mL) was heated to 100-105° C. to a solution. The mixture was cooled to 75-85° C. over 90 min and agitated for 1 h. The mixture was cooled to 20-25° C. over 2 h and agitated for 2 h. The solids were isolated and rinsed with toluene (40 mL). The recrystallization process was repeated one more time.

Free base: to a mixture of the wet cake in toluene (225 mL) and water (225 mL) was added 30% aq. NaOH at 10-15° C. to pH 9-10. The mixture was agitated for 30 min and the organic phase was separated. To the aqueous phase was added 6 M aq. HCl at 10-15° C. to pH 2-3. The mixture was then cooled to 3-8° C. and agitated for 1 h. The solids were isolated and washed with water (40 mL). The wet cake was dried under vacuum at 50-55° C. to give (1R,4S,5S)-2-(tert-butoxycarbonyl)-2-azabicyclo[2.1.1]hexane-5-carboxylic acid (25 g, 18% yield).

A mixture of the acid (245 g), pyridine (86 g) and ammonium carbonate (111 g) in MeCN (3700 mL) was added (Boc)₂O (310 g) at 15-25° C. The mixture was agitated for 5 h to complete the reaction. The solids were isolated and rinsed with MeCN (250 mL). The filtrate and rinse were combined and concentrated under vacuum at 40-45° C. and azeotroped with heptane. To the residue was added EtOAc (130 mL) and n-heptane (650 mL) at 40-45° C. The mixture was cooled to 10-15° C. and agitated for 2 h. The solids were isolated and rinsed with n-heptane (250 mL). The wet cake was dried under vacuum at 50-55° C. to give tert-butyl (1R,4S,5S)-5-carbamoyl-2-azabicyclo[2.1.1]hexane-2-carboxylate quantitatively.

To cooled 15% aq. NaOH (800 mL) at 10-15° C. was added the tert-butyl (1R,4S,5S)-5-carbamoyl-2-azabicyclo[2.1.1]hexane-2-carboxylate (214 g). Sodium hypochlorite (91.2 g) was added at 10-20° C. and the mixture was agitated for 2 h. The mixture was heated to 40-45° C. for 4 h to complete the reaction. The reaction mixture was cooled to 15-20° C. and citric acid was added to adjust pH 5-6. The mixture was basified by addition of NaOH to pH 14. The basified mixture was extracted with 2-MeTHF (2×1000 mL). The combined organic phase was concentrated under vacuum and the residual was azeotroped with MeCN. The residue was dissolved in MeCN (140 mL) and activated charcoal (2 gram) was added. The mixture was agitated at 25-30° C. for 2 h. The mixture was filtered, and the filter bed was rinsed with MeCN (85 mL). The combined filtrate and rinse were added to a solution of oxalic acid (120 g) in MeCN (850 mL) at 40-45° C. The solution was cooled to 3-7° C. and agitated for 1 h. The solids were isolated and rinsed with MeCN (110 mL). The wet cake was dried at 40-50° C. under vacuum to give tert-butyl (1R,4R,5S)-5-amino-2-azabicyclo[2.1.1]hexane-2-carboxylate oxalate (248 g, 91% yield) as a white solid. LCMS for calc. C₁₀H₁₈N₂O₂: 198.14; Found (M+H): 199.1 ¹H NMR (500 MHz, DMSO-d₆): δ8.44 (s, 3H); 3.34, (m, 1H); 4.24, dt, 1H, J=6.9, 1.7 Hz); 3.20-3.31 (m, 2H); 2.84, (dt, 1H, J=6.5, 3.0); 1.65-1.71 (m, 1H); 1.42 (s, 9H); 1.19 (d, 1H, J=8.1). ¹³C NMR (125 Hz, DMSO-d₆): b 165.0; 155.8; 79.5; 61.5; 50.6; 44.9; 40.8; 33.8; 28.6.

### Example 8. Additional Salts

### Characterization Parameters

### Hemifumarate (Compound 1 Hemifumarate)

The preparation of Compound 1 hemifumarate and Compound 1 hemifumarate hemimethanol is shown in Example 2.

### Example 8a. (1R,3R,4R,5S)-3-((R)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate dihydrochloride (Compound 1 Di-hydrochloride)

Preparation: 133.70 mg of free base was dissolved in MeOH (0.2 mL) and EtOAc (3 mL) in a 4 mL clear glass vial with stirring. To the solution, aq. 6 M HCl (49.7 pL) was added with stirring to solid out. The resulted suspension was stirred at r.t. for 1 h. The solid was collected by vacuum filtration, washed with EtOAc and vacuum dried at 50° C. for 1 h. The salt ratio between hydrochloric acid free base is determined to be 1.99 by chloride titration.

Di-hydrochloride salt was confirmed as a crystalline solid according to XRPD analysis. The XRPD pattern is shown in FIG. 4 and the peak data are provided in Table 2.

The DSC thermogram is shown in FIG. 5. It dehydrated first below 150° C. and then followed by exothermal melting/decomposition at an onset temperature of 217.9° C. with a peak temperature of 260.9° C.

The TGA thermogram is shown in FIG. 6. Weight loss of ˜4.3% was observed below 150° C. due to loss of water. The compound decomposes above 150° C. with weight loss of ˜23.8% up to 300° C.

### Example 8b. (1R,3R,4R,5S)-3-((R)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hydrochloride (Compound 1 Hydrochloride)

Preparation: 89.45 mg of free base was dissolved in MeOH (0.2 mL) in a 4 mL clear glass vial with stirring. To the solution, aq. 6 M HCl (22.4 μL) was added with stirring to solid out quickly. Then EtOAc (1 mL) was added. The resulting suspension was stirred at r.t. for 1 h. The solid was collected by vacuum filtration, washed with EtOAc and vacuum dried at 50° C. for 1 h. The salt ratio between hydrochloric acid free base is determined to be 1.05 by chloride titration.

Compound 1 hydrochloride was confirmed as a crystalline solid according to XRPD analysis. The XRPD pattern is shown in FIG. 7 and the peak data are provided in Table 3.

The DSC thermogram is shown in FIG. 8. It de-hydrated first below 150° C. and then followed by exothermal melting/decomposition at an onset temperature of 269.2° C. with a peak temperature of 274.9° C.

The TGA thermogram is shown in FIG. 9. Weight loss of ˜2.8% was observed below 140° C. due to loss of water. The compound decomposes above 140° C. with weight loss of ˜21.20% up to 300° C.

### Example 8c. (1R,3R,4R,5S)-3-((R)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate L-tartrate (Compound 1 L-Tartrate)

Preparation: 82.33 mg of free base was dissolved in acetone (2.5 mL) in a 4 mL clear glass vial with heating at 50° C. and stirring. To the solution, L-tartaric acid (21.86 mg) was added and mixed well. The solution was evaporated without cap at r.t. to semi-solid. Then the semi-solid was dissolved in MeOH (0.2 mL) and EtOAc (2 mL). The solution was stirred at r.t. for 3 d to solid out. The solid of L-tartrate salt was collected by filtration, washed with EtOAc and vacuum dried at 50° C. for 1 h. The salt ratio between the free base and L-tartaric acid was determined to be 1.36 by NMR analysis.

Compound 1 L-tartrate salt was confirmed as a crystalline solid according to XRPD analysis. The XRPD pattern is shown in FIG. 10 and the peak data are provided in Table 4.

The DSC thermogram is shown in FIG. 11. It de-hydrated first below 125° C. and then followed by melting/decomposition at an onset temperature of 210.5° C. with a peak temperature of 215.8° C.

The TGA thermogram is shown in FIG. 12. Weight loss of ˜1.0% was observed below 125° C. mainly due to loss of water. The compound decomposes above 125° C. with weight loss of ˜23.6% up to 300° C.

### Example 8d. (1R,3R,4R,5S)-3-((R)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate phosphate (Compound 1 Phosphate)

Preparation: 92.37 mg of free base was dissolved in MeOH (1 mL) in a 4 mL clear glass vial with stirring. To the solution, 85% phosphoric acid (10 μL) was added with stirring. The solution was evaporated without cap at r.t. overnight. To the resulted solution, acetone (1 mL) was added and evaporated without cap at r.t. overnight to solid out. Then MeOH (0.2 mL) and EtOAc (0.5 mL) were added. The suspension was stirring at r.t. for 1 h. Phosphate salt was collected by filtration, washed with EtOAc and vacuum dried at 50° C. for 1 h. The salt ratio between the free base and phosphoric acid was determined to be 1.0 by NMR analysis.

Compound 1 phosphate was confirmed as a crystalline solid according to XRPD analysis. The XRPD pattern is shown in FIG. 13 and the peak data are provided in Table 5.

The DSC thermogram is shown in FIG. 14. It de-hydrated first below 100° C. and then followed by exothermal melting/decomposition at an onset temperature of 254.7° C. with a peak temperature of 256.9° C.

The TGA thermogram is shown in FIG. 15. Weight loss of 1.4% was observed below 140° C. The second weight loss of ˜15.2% between 140-300° C. is due to decomposition of the compound.

### Example 9. Compound 1 Hemifumarate Tablet Formulation

Manufacturing Procedure for Compound 1-hemifumarate 100 mg Tablet

Step 1: Pass Compound 1 hemifumarate through a suitable screen into blender.

Step 2: Pass colloidal silicon dioxide (intragranular portion), sodium starch glycolate (intragranular portion), microcrystalline cellulose, and lactose anhydrous through a suitable screen and add to blender and mix.

Step 3: Pass magnesium stearate (intragranular portion) through a suitable screen and add to blender and mix.

Step 4: Feed intragranular blend from Step 3 into the roller compactor and mill resulting ribbons into granules with a mill.

Step 5: Split the milled granules into two halves. Transfer half of the milled granules to a suitable blender.

Step 6: Pass colloidal silicon dioxide (extragranular portion), sodium starch glycolate (extragranular portion) through a suitable screen and add to blender from Step 5

Step 7: Add remaining half of the milled granules to blender from Step 6 and mix.

Step 8: Pass sodium stearyl fumarate through a suitable screen and add to blender from Step 7 and mix

Step 9: Pass magnesium stearate (extragranular portion) through a suitable screen and add to blender from Step 8 and mix

Step 10: Compress the blend from Step 9 on a suitable rotary tablet machine

The tablet formulation was prepared using the components and amounts listed in Table 6.

TABLE 6

| | | 100 mg Strength | |
|---|---|---|---|
| | | Formulation | |
| | | | Composition |
| Component | Function | mg/tablet | (wt %) |

Intragranular Material

| | | | |
|---|---|---|---|
| Compound 1 hemifumarate^{a} | Active | 108.29 | 15.04 |
| (Drug Substance) | | (100.00^{b}) | |
| Microcrystalline Cellulose, | Diluent | 278.50 | 38.68 |
| USP/Ph. Eur./JP | | | |
| Lactose Anhydrous, NF/Ph. | Diluent | 278.50 | 38.68 |
| Eur./JP | | | |
| Sodium Starch Glycolate, | Disintegrant | 14.40 | 2.0 |
| USP/Ph. Eur./JP/CHP | | | |
| Colloidal Silicone Dioxide, | Glidant | 2.16 | 0.30 |
| USP/Ph. Eur./JP/CHP | | | |
| Magnesium Stearate, NF/JP/BP | Lubricant | 3.60 | 0.50 |

| | | |
|---|---|---|
| Total Intragranular Materials | 685.44 | 95.2 |

Extragranular Material

| | | | |
|---|---|---|---|
| Sodium Starch Glycolate, | Disintegrant | 21.60 | 3.0 |
| NF/Ph. Eur./JP | | | |
| Colloidal Silicone Dioxide, | Glidant | 2.16 | 0.30 |
| USP/Ph. Eur./JP/CHP | | | |
| Sodium Stearyl Fumarate, | Lubricant | 7.20 | 1.0 |
| NF/Ph. Eur./JP | | | |
| Magnesium Stearate, NF/JP/BP | Lubricant | 3.60 | 0.50 |

| | | |
|---|---|---|
| Total Extragranular Materials | 34.56 | 4.8 |
| Tablet Total | 720.0 | 100.0 |

^{a}Salt to free base conversion factor = 0.9233
^{b}Free base equivalent

An additional tablet formulation was prepared for a 200 mg Compound 1 (free base equivalent) tablet using the components and amounts listed in Table 7.

TABLE 7

| | | 200 mg Strength | |
|---|---|---|---|
| | | Formulation | |
| | | | Composition |
| Component | Function | mg/tablet | (wt %) |

Intragranular Material

| | | | |
|---|---|---|---|
| Compound 1 hemifumarate^{a} | Active | 216.58 | 30.08 |
| (Drug Substance) | | (200.00^{b}) | |
| Microcrystalline Cellulose, | Diluent | 224.35 | 31.16 |
| USP/Ph. Eur./JP | | | |
| Lactose Anhydrous, NF/Ph. | Diluent | 224.35 | 31.16 |
| Eur./JP | | | |
| Sodium Starch Glycolate, | Disintegrant | 14.40 | 2.0 |
| USP/Ph. Eur./JP/CHP | | | |
| Colloidal Silicone Dioxide, | Glidant | 2.16 | 0.30 |
| USP/Ph. Eur./JP/CHP | | | |
| Magnesium Stearate, NF/JP/BP | Lubricant | 3.60 | 0.50 |

| | | |
|---|---|---|
| Total Intragranular Materials | 685.44 | 95.2 |

Extragranular Material

| | | | |
|---|---|---|---|
| Sodium Starch Glycolate, | Disintegrant | 21.60 | 3.0 |
| NF/Ph. Eur./JP | | | |
| Colloidal Silicone Dioxide, | Glidant | 2.16 | 0.30 |
| USP/Ph. Eur./JP/CHP | | | |
| Sodium Stearyl Fumarate, | Lubricant | 7.20 | 1.0 |
| NF/Ph. Eur./JP | | | |
| Magnesium Stearate, NF/JP/BP | Lubricant | 3.60 | 0.50 |

| | | |
|---|---|---|
| Total Extragranular Materials | 34.56 | 4.8 |
| Tablet Total | 720.0 | 100.0 |

^{a}Salt to free base conversion factor = 0.9233
^{b}Free base equivalent

### Example 10. Solubility Studies of (1R,3R,4R,5S)-3-((R,)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate Hemifumarate (Compound 1 Hemifumarate)

At 25° C., Compound 1 hemifumarate exhibited excellent solubility in (50 mg/mL) in DMF, 2-methoxyethanol, DMSO, and the mixture of two solvent system of DCM and MeOH (volume ratio v/v DCM:MeOH=4:1 and 1:1). It is soluble (15 mg/mL≤solubility<50 mg/mL) in CHCl₃, MeOH, and THF. It is slightly soluble (1 mg/mL≤solubility<15 mg/mL) in MeCN, DCM, 1,4-dioxane, acetone, methyl ethyl ketone (MEK), methyl iso-butyl ketone, EtOH, n-propanol, n-BuOH, water, EtOAc, iso-butyl acetate, iso-propyl acetate, ethyl formate, 2-methyl tetrahydrofuran (2-MeTHF), and the mixture of the two solvents of EtOH and MeOH [weight ratio, 1% (w/w), 5% (w/w) and 10% (w/w) MeOH in EtOH]. It has poor solubility (<1 mg/mL) in MTBE, a mixture of two solvent system (v/v MTBE:MeOH=10:1 and 1:1), toluene, o-xylene, and iso-propanol (IPA). It is completely insoluble in heptane.

At 50° C., Compound 1 hemifumarate exhibited excellent solubility in (50 mg/mL) in DMF, 2-methoxyethanol, DMSO, THF, 1,4-dioxane, and the mixture of two solvent system of DCM and MeOH (volume ratio v/v DCM:MeOH=4:1 and 1:1). It is soluble (15 mg/mL≤solubility<50 mg/mL) in DCM, CHCl₃, MeOH, MEK, and ethyl formate. It is slightly soluble (1 mg/mL≤solubility<15 mg/mL) in MeCN, acetone, methyl iso-butyl ketone, EtOH, n-propanol, n-BuOH, water, EtOAc, iso-butyl acetate, iso-propyl acetate, and the mixture of the two solvents of EtOH and MeOH [weight ratio, 1% (w/w), 5% (w/w) and 10% (w/w) MeOH in EtOH] and 2-MeTHF. It has poor solubility (<1 mg/mL) in MTBE, a mixture of two solvent system (v/v MTBE:MeOH=10:1 and 1:1), toluene, o-xylene, and IPA. It is completely insoluble in heptane. These results are shown in Table 8.

TABLE 8

| Ex. No. | Solvent | 25° C. | 50° C. |
|---|---|---|---|
| 1 | MeCN | 4.8 | 6.5 |
| 2 | Chloroform | 22.8 | 30.4 |
| 3 | DCM | 13.1 | 18.2 |
| 3A | DCM:MeOH (4:1, v/v) | >50 | >50 |
| 3B | DCM:MeOH (1:1, v/v) | >50 | >50 |
| 4 | DMF | >50 | >50 |
| 5 | 1,4-Dioxane | 11.5 | >50 |
| 6 | MeOH | 16.8 | 26.1 |
| 7 | 2-Methoxyethanol | >50 | >50 |
| 8 | Methyl iso-butyl ketone | 1.2 | 3.9 |
| 9 | Toluene | 0.4 | 0.6 |
| 10 | Acetone | 2.2 | 4.8 |
| 11 | n-BuOH | 1.0 | 4.7 |
| 12 | MTBE | 0.3 | 0.6 |
| 12A | MTBE:MeOH (10:1, v/v) | 0.4 | 0.6 |
| 12B | MTBE:MeOH (1:1, v/v) | 0.6 | 0.9 |
| 13 | DMSO | >50 | >50 |
| 14 | EtOH | 1.9 | 3.1 |
| 14A | 1% MeOH in EtOH (w/w) | 2.0 | 3.2 |
| 14B | 5% MeOH in EtOH (w/w) | 2.1 | 3.4 |
| 14C | 10% MeOH in EtOH (w/w) | 2.4 | 3.9 |
| 15 | EtOAc | 3.3 | 4.7 |
| 16 | Ethyl formate | 7.6 | 45.5 |
| 17 | Heptane | NA | NA |
| 18 | iso-Butyl acetate | 1.5 | 2.4 |
| 19 | iso-Propyl acetate | 1.6 | 2.5 |
| 20 | n-Propanol | 1.2 | 2.1 |
| 21 | IPA | 0.5 | 0.8 |
| 22 | Water | 2.3 | 2.9 |
| 23 | MEK | 8.6 | 35.4 |
| 24 | THF | 22.7 | >50 |
| 25 | 2-Methyl THF | 4.8 | 6.8 |
| 26 | o-Xylene | 0.1 | 0.2 |

### Example 11. Polymorph Studies of Crystalline (1R,3R,4R,5S)-3-((R,)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate phosphate (Compound 1 Hemifumarate)

### Phase Equilibration

Phase equilibration studies were designed to provide information on a predominant crystal form for phase identification. Based on its solubility in various solvent systems (Table 9), Compound 1 hemifumarate was equilibrated in the representative groups of solvents at 25±1° C. (Table 10) and 50±1° C. (Table 11). To the solvents listed in Table 10 and Table 11, Compound 1 hemifumarate was added until a cloudy solution was obtained, then, approximately 20 mg of Compound 1 hemifumarate was added to the cloudy solution. The mixture was stirred at 25±1° C. and 50±1° C. for 48 h and 24 h, respectively. The solid was filtered, dried in vacuum, and analyzed by XRPD to give the results in Table 9 and Table 10. Only Form I was observed, and no new form was obtained.

TABLE 9

| Exp. No. | Solvent | Solid Form |
|---|---|---|
| 1 | EtOH:MeOH (v/v, 10:1) | I |
| 2 | MeCN | I |
| 3 | Chloroform | I |
| 4 | DCM | I |
| 5 | DMF | I |
| 6 | 1,4-Dioxane | I |
| 7 | MeOH | I |
| 8 | 2-Methoxy-ethanol | NA |
| 9 | MIBK | I |
| 10 | Toluene | I |
| 11 | Acetone | I |
| 12 | n-BuOH | I |
| 13 | MTBE | I |
| 14 | MTBE:MeOH (10:1, v/v) | I |
| 15 | MTBE:MeOH (1:1, v/v) | I |
| 16 | DMSO | I |
| 17 | EtOH | I |
| 18 | 1% MeOH in EtOH (w/w) | I |
| 19 | 5% MeOH in EtOH (w/w) | I |
| 20 | 10% MeOH in EtOH (w/w) | I |
| 21 | EtOAc | I |
| 22 | Ethyl formate | I |
| 23 | Heptane | I |
| 24 | Isobutyl acetate | I |
| 25 | IPAc | I |
| 26 | n-Propanol | I |
| 27 | IPA | I |
| 28 | Water | I |
| 29 | MEK | I |
| 30 | THF | I |
| 31 | 2-Methyl THF | I |
| 32 | o-Xylene | I |

TABLE 10

| Exp. No. | Solvent | Solid Form |
|---|---|---|
| 1 | MeCN | I |
| 2 | Chloroform | I |
| 3 | DCM | I |
| 4 | DMF | NA |
| 5 | 1,4-Dioxane | NA |
| 6 | MeOH | I |
| 7 | 2-Methoxyethanol | NA |
| 8 | MIBK | I |
| 9 | Toluene | I |
| 10 | Acetone | I |
| 11 | n-BuOH | I |
| 12 | MTBE | I |
| 13 | MTBE:MeOH (10:1, v/v) | I |
| 14 | MTBE:MeOH (1:1, v/v) | I |
| 15 | DMSO | NA |
| 16 | EtOH | I |
| 17 | 1% MeOH in EtOH (w/w) | I |
| 18 | 5% MeOH in EtOH (w/w) | I |
| 19 | 10% MeOH in EtOH (w/w) | I |
| 20 | EtOAc | I |
| 21 | Ethyl formate | I |
| 22 | Heptane | I |
| 23 | Isobutyl acetate | I |
| 24 | IPAc | I |
| 25 | n-Propanol | I |
| 26 | IPA | I |
| 27 | Water | I |
| 28 | MEK | I |
| 29 | THF | I |
| 30 | 2-Methyl THF | I |
| 31 | o-Xylene | I |

### Evaporation

Evaporation studies were performed to identify the predominant crystalline form during uncontrolled evaporation. XRPD was used to study the solid-state morphology of the crystalline forms of the evaporation samples at 25±1° C. and 50±1° C. The results are presented in Table 11 (25±1° C.) and Table 12 (50±1° C.).

TABLE 11

| Exp. No. | Solvent | Solid Form |
|---|---|---|
| 1 | MeCN | I |
| 2 | Chloroform | Amorphous |
| 3 | DCM | Amorphous + I |
| 4 | DCM:MeOH 4:1 | I |
| 5 | DCM:MeOH 1:1 | I |
| 6 | DMF | NA |
| 7 | 1,4-Dioxane | Amorphous |
| 8 | MeOH | I |
| 9 | 2-Methoxyethanol | NA |
| 10 | MIBK | NA |
| 11 | Toluene | NA |
| 12 | Acetone | NA |
| 13 | n-BuOH | NA |
| 14 | MTBE | NA |
| 15 | MTBE:MeOH (10:1, v/v) | NA |
| 16 | MTBE:MeOH (1:1, v/v) | I |
| 17 | DMSO | NA |
| 18 | EtOH | NA |
| 19 | 1% MeOH in EtOH (w/w) | NA |
| 20 | 5% MeOH in EtOH (w/w) | I |
| 21 | 10% MeOH in EtOH (w/w) | I |
| 22 | EtOAc | Amorphous + I |
| 23 | Ethyl formate | Amorphous |
| 24 | Heptane | NA |
| 25 | Isobutyl acetate | NA |
| 26 | IPAc | NA |
| 27 | n-Propanol | NA |
| 28 | IPA | NA |
| 29 | Water | NA |
| 30 | MEK | Amorphous |
| 31 | THF | Amorphous + I |
| 32 | 2-Methyl THF | I |
| 33 | o-Xylene | NA |

N/A: Not available.
The amount of the precipitate was too small to be analyzed by XRPD.

TABLE 12

| Exp. No. | Solvent | Solid Form |
|---|---|---|
| 1 | MeCN | Amorphous |
| 2 | Chloroform | Amorphous |
| 3 | DCM | Amorphous + I |
| 4 | DCM:MeOH (4:1, v/v) | I |
| 5 | DCM:MeOH (1:1, v/v) | I |
| 6 | DMF | NA |
| 7 | 1,4-Dioxane | NA |
| 8 | MeOH | I |
| 9 | 2-Methoxyethanol | NA |
| 10 | MIBK | NA |
| 11 | Toluene | NA |
| 12 | Acetone | NA |
| 13 | n-BuOH | I |
| 14 | MTBE | NA |
| 15 | MTBE:MeOH (10:1, v/v) | I |
| 16 | MTBE:MeOH (1:1, v/v) | I |
| 17 | DMSO | NA |
| 18 | EtOH | I |
| 19 | 1% MeOH in EtOH (w/w) | I |
| 20 | 5% MeOH in EtOH (w/w) | I |
| 21 | 10% MeOH in EtOH (w/w) | I |
| 22 | EtOAc | Amorphous |
| 23 | Ethyl formate | Amorphous |
| 24 | Heptane | NA |
| 25 | Isobutyl acetate | Amorphous |
| 26 | IPAc | Amorphous |
| 27 | n-Propanol | I |
| 28 | IPA | NA |
| 29 | Water | NA |
| 30 | MEK | NA |
| 32 | THF | Amorphous + I |
| 33 | 2-Methyl THF | I |
| 34 | o-Xylene | NA |

N/A: Not available.
Either the amount of the precipitate was too small to be analyzed by XRPD or the solids were sticky.

### Antisolvent Addition

On the basis of solubility data in Table 9, the solvents of DMF, 2-methoxyethanol, and DCM:MeOH (4:1, v/v) were chosen to dissolve the sample while the solvents of MTBE and IPA were chosen for anti-solvent.

Saturated or nearly saturated solutions of Compound 1 hemifumarate (Form I) (˜300 mg/mL in DMF, ˜90 mg/mL in 2-methoxyethanol, and ˜300 mg/mL DCM:MeOH (4:1, v/v)) were prepared in the solvents listed in Table 12 at r.t. respectively. An anti-solvent was added dropwise to induce precipitation. The results are presented in Table 13. No new form was obtained.

TABLE 13

| | | | Solid |
|---|---|---|---|
| Exp. No. | Solvent (mL) | Anti-solvent (mL) | Form |
| 1 | DCM:MeOH (4:1, v/v) | EtOH | I |
| 2 | DMF (1.0) | MTBE (8.0) | I |
| 3 | DMF (1.0) | IPA (8.0) | I |
| 4 | 2-Methoxyethanol (1.0) | MTBE (8.0) | NA |
| 5 | 2-Methoxyethanol (1.0) | IPA (8.0) | NA |
| 6 | DCM:MeOH (4:1, v/v) (1.0) | MTBE (8.0) | I |
| 7 | DCM:MeOH (4:1, v/v) (1.0) | IPA (8.0) | I |

N/A: Not available.
Either no solids precipitated or sticky solids, which are not analyzed by XRPD.

### Reverse Addition

Saturated or nearly saturated solutions of Compound 1 hemifumarate (Form 1) (˜300 mg/mL in DMF, ˜90 mg/mL in 2-methoxyethanol, and ˜300 mg/mL DCM:MeOH (4:1, v/v) were prepared in the solvents listed in Table 14 at r.t. and added dropwise to a larger volume of a miscible anti-solvent. The results are presented in Table 14. In reverse addition experiments, no new polymorphic form was obtained.

TABLE 14

| | | | Solid |
|---|---|---|---|
| Exp. No. | Solvent (mL) | Anti-solvent (mL) | Form |
| 1 | DMF (1.0) | MTBE (8.0) | I |
| 2 | DMF (1.0) | IPA (8.0) | I |
| 3 | 2-Methoxyethanol (1.0) | MTBE (8.0) | NA |
| 4 | 2-Methoxyethanol (1.0) | IPA (8.0) | NA |
| 5 | DCM:MeOH (4:1, v/v) (1.0) | MTBE (8.0) | I |
| 6 | DCM:MeOH (4:1, v/v) (1.0) | IPA (8.0) | I |

N/A: Not available.
Either no solids precipitated or sticky solids, which are not analyzed by XRPD.

### Quench Cool of Saturated Solution

Saturated or nearly saturated solutions of Compound 1 hemifumarate (Form 1) were prepared at 25° C. DMF, 2-methoxyethanol, DCM:MeOH (4:1, v/v) solvents were chosen for this study on the basis of the respective solubility data (the DMF solution: ˜300 mg/mL; the 2-methoxyethanol solution: ˜90 mg/mL; and the DCM:MeOH solution: ˜300 mg/mL). These solutions were quenched cooled to about ˜70° C. to induce precipitation of higher energy forms. No precipitation was observed, but viscous mixtures were observed (no solids were obtained) in these experiments.

As can be seen, all the crystalline solids of Compound 1 hemifumarate obtained were Form I. No new crystalline forms of Compound 1 hemifumarate were found during the polymorph screening. This suggests that Form I is the low-energy form of Compound 1 hemifumarate.

### Example 11. Characterization of (1R,3R,4R,5S)-3-((Rₐ)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate Hemifumarate Hemimethanol (Compound 1 Hemifumarate Hemimethanol)

### Data Collection

A colorless block having approximate dimensions of 0.2×0.12×0.08 mm³, was mounted on a polymer loop in random orientation. Preliminary examination and data collection were performed on a Rigaku SuperNova diffractometer, equipped with a copper anode microfocus sealed X-ray tube (Cu Kα λ=1.54184 Å) and a Dectris Pilatus3 R 200K hybrid pixel array detector.

Cell constants and an orientation matrix for data collection were obtained from least-squares refinement using the setting angles of 17440 reflections in the range 4.0960°<θ<75.5110°. The space group was determined by the program CRYSALISPRO to be P2₁2₁2 (international tables no. 18). The data were collected to a maximum diffraction angle (20) of 151.914° at r.t.

### Data Reduction

Frames were integrated with CRYSALISPRO. A total of 35586 reflections were collected, of which 14504 were unique. Lorentz and polarization corrections were applied to the data. The linear absorption coefficient is 2.212 mm⁻¹ for Cu Kα radiation. An empirical absorption correction using CRYSALISPRO was applied. Transmission coefficients ranged from 0.849 to 1.000. Intensities of equivalent reflections were averaged. The agreement factor for the averaging was 2.92% based on intensity.

### Structure Solution and Refinement

The structure was solved by direct methods using SHELXT. The remaining atoms were located in succeeding difference Fourier syntheses. The structure was refined using SHELxL-2014. Hydrogen atoms residing on nitrogen were refined independently. All other hydrogen atoms were included in the refinement but restrained to ride on the atom to which they are bonded. The structure was refined in full-matrix least-squares by minimizing the function:

$$\sum {w ( {\vert {F}_{o} \vert}^{2} - {\vert {F}_{c} \vert}^{2} )}^{2}$$

where the weight, w, is defined as 1/[σ²(Fₒ²)⁺(0.1140P)²+(2.1927P)], where P=(Fₒ²+2F_{c}²)/3.

Scattering factors were taken from the “International Tables for Crystallography.” Of the 14504 reflections used in the refinements, only the reflections with intensities larger than twice their uncertainty [I>2σ(I)], 13170, were used in calculating the fit residual, R. The final cycle of refinement included 987 variable parameters, 0 restraints, and converged with respective unweighted and weighted agreement factors of:

$$R = Σ \vert {F}_{o} - {F}_{c} \vert / {ΣF}_{o} = 0 \text{.0574}$$

$${R}_{w} = \sqrt{( {Σw ( {F}_{o}^{2} - {F}_{c}^{2} )}^{2} / {Σw ( {F}_{o}^{2} )}^{2} )} = 0.1722$$

The standard deviation of an observation of unit weight (goodness of fit) was 1.05. The highest peak in the final difference Fourier had an electron density of 0.572 e/Å³. The minimum negative peak had a value of −0.444 e/Å³.

### Calculated XRPD

A calculated XRPD pattern (FIG. 16) was generated for Cu radiation using MERCURY and the atomic coordinates, space group, and unit cell parameters from the single crystal structure.

### Atomic Displacement Ellipsoid and Packing Diagrams

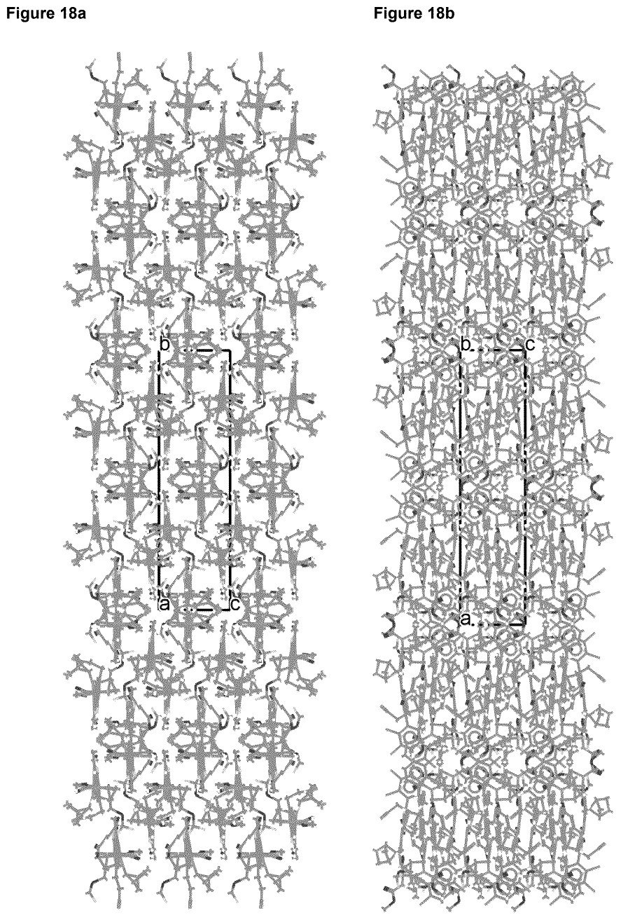

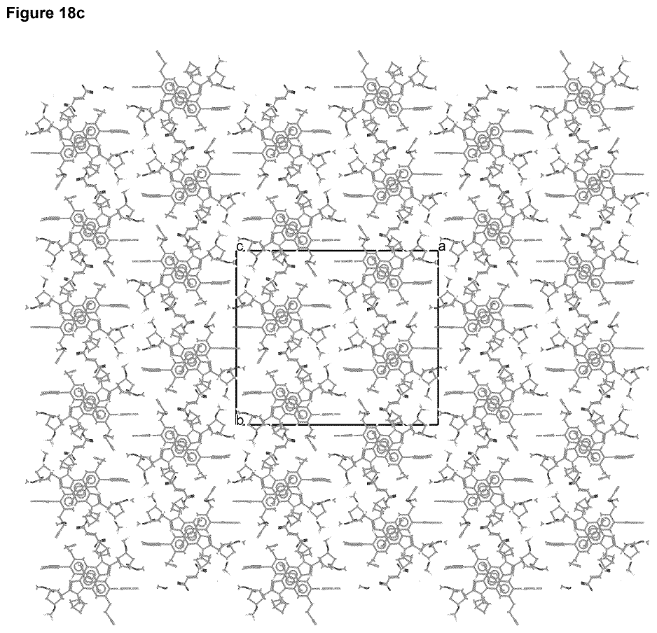

The atomic displacement ellipsoid diagram was prepared using MERCURY (FIG. 17). Atoms are represented by 50% probability anisotropic thermal ellipsoids. The packing diagrams (FIGS. 18a, 18b, and 18c) were generated with MERCURY. Hydrogen bonding is represented as dashed lines. Assessment of chiral centers was performed with PLATON. Absolute configuration is evaluated using the specification of molecular chirality rules.

As can be seen, the single crystal structure of Compound 1 hemifumarate hemimethanol was determined to confirm the molecular structure and absolute configuration. The structure of Compound 1 hemifumarate hemimethanol was determined to be a methanol solvated crystal form, composed of two Compound 1 cations, one fumarate anion, and one methanol molecule in the asymmetric unit. The absolute structure was determined from the crystal structure and the molecule was found to bond in the S, R, R, R, R, R, and S configuration at C22, C23, C25, C27, C28, C30, and C32, respectively. The atropisomeric arrangement of the biphenyl rings is in the M (Rₐ) configuration.

Various modifications of the disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A process for preparing a compound of Formula I: 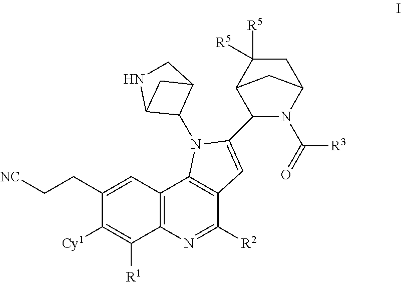
wherein
Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
R¹ is halogen;
R² is C₁₋₃ alkyl optionally substituted with OH;
R³ is OR^{3A};
R^{3A} is selected from C₁₋₃ alkyl, C₂₋₃ alkenyl, and C₂₋₃ alkynyl;
each R⁵ is independently selected from H, D, halo, C₁₋₃ alkyl, OR^{a5}, C₁₋₃ haloalkyl, C₂₋₃ alkenyl, and C₂₋₃ alkynyl; and
each R^{a5} is independently selected from H, C₁₋₃ alkyl, and C₁₋₃ haloalkyl;
comprising deprotecting a compound of Formula II: 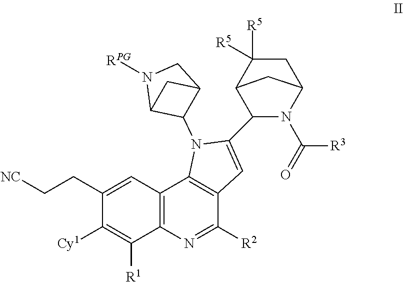
wherein
R^{PG} is a nitrogen protecting group;
to produce the compound of Formula I.

2-6. (canceled)

7. The process of claim 1, wherein the process further comprises preparing a compound of any one of Formulae II, III, IV, VI, VII, VIII, X, XI, XII, XIII, XV, XVI, XVII, and XVIII, wherein:
(1) the compound prepared is a compound of Formula II: 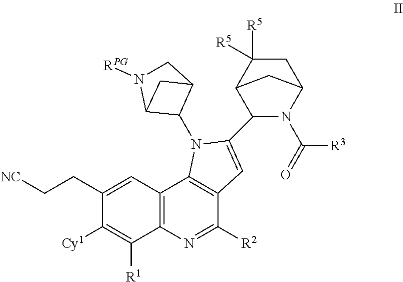
wherein
R^{PG} is a nitrogen protecting group;
Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
R¹ is halogen;
R² is C₁₋₃ alkyl optionally substituted with OH;
R³ is OR^{3A};
R^{3A} is selected from C₁₋₃ alkyl, C₂₋₃ alkenyl, and C₂₋₃ alkynyl;
each R⁵ is independently selected from H, D, halo, C₁₋₃ alkyl, OR^{a5}, C₁₋₃ haloalkyl, C₂₋₃ alkenyl, and C₂₋₃ alkynyl; and
each R^{a5} is independently selected from H, C₁₋₃ alkyl, and C₁₋₃ haloalkyl;
and the process comprises cyclizing a compound of Formula III: 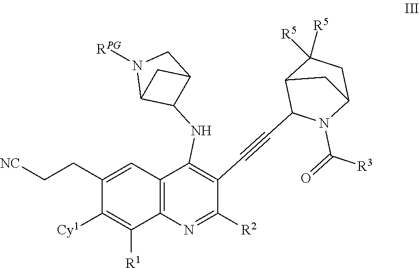
to form the compound of Formula II;
(2) the compound prepared is a compound of Formula III: 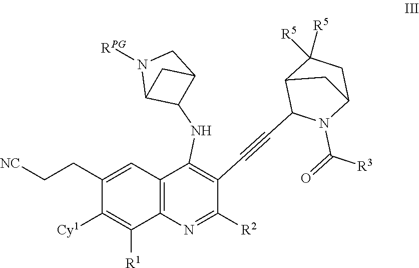
and the process comprises:
coupling a compound of Formula IV: 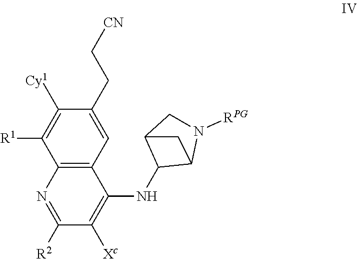
with a compound of Formula V: 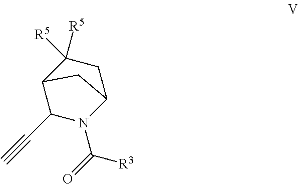
to form the compound of Formula III
wherein
X^{c} is Cl, Br, or I;
(3) the compound prepared is a compound of Formula IV: 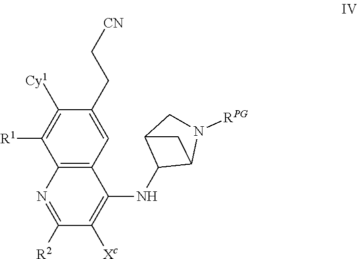
and the process comprises halogenating a compound of Formula VI: 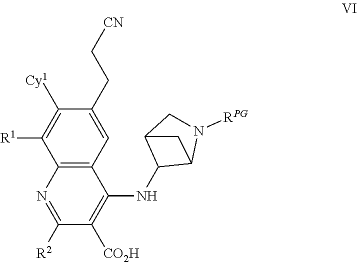
wherein
to form the compound of Formula IV;
(4) the compound prepared is a compound of Formula VI: 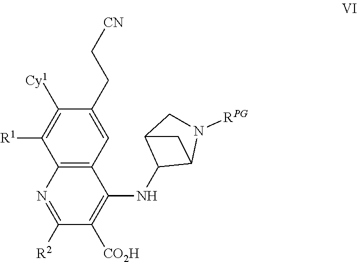
and the process comprises hydrolyzing a compound of Formula VII: 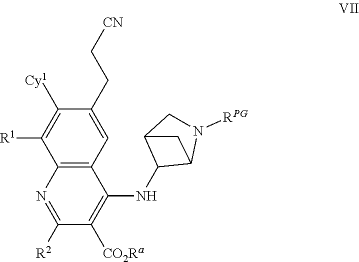
wherein
R^{a} is C₁₋₃ alkyl;
to form the compound of Formula VI:
(5) the compound prepared is a compound of Formula VII: 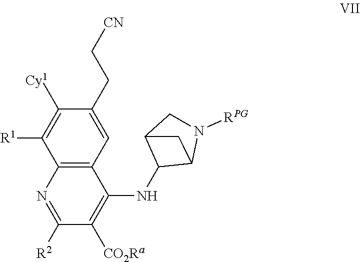
and the process comprises reacting a compound of Formula VIII: 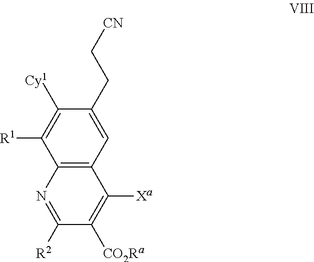
wherein
X^{a} is halo or OH;
with a compound of Formula IX: 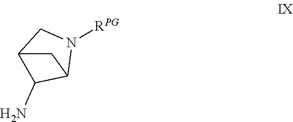
to form the compound of Formula VII;
(6) the compound prepared is a compound of Formula VIII: 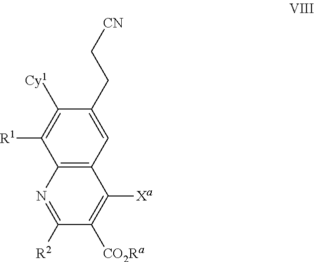
and the process comprises reducing a compound of Formula X: 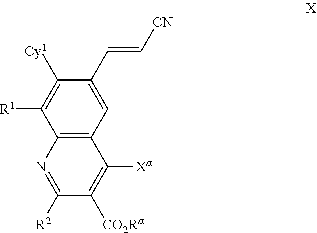
to form the compound of Formula VIII:
(7) the compound prepared is a compound of Formula X: 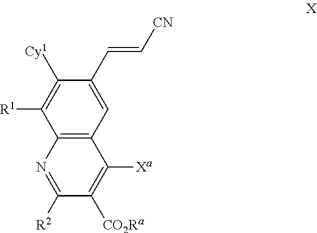
and the process comprises halodehydroxylating a compound of Formula XI: 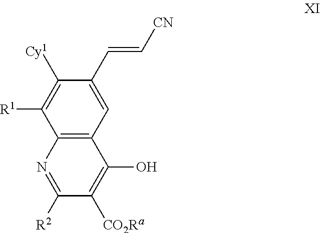
to form the compound of Formula X;
(8) the compound prepared is a compound of Formula XI: 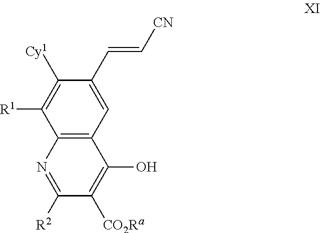
and the process comprises coupling a compound of Formula XII: 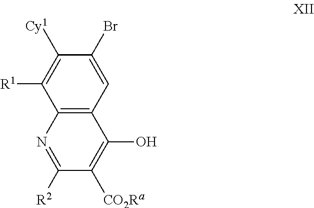
with an alkene form the compound of Formula XI:
(9) the compound prepared is a compound of Formula XII: 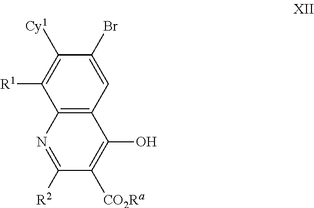
and the process comprises reacting a compound of Formula XIII: 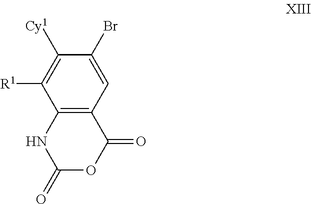
with a compound of Formula XIV: 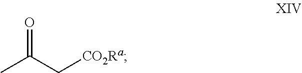
to form the compound of Formula XII:
(10) the compound prepared is a compound of Formula XIII: 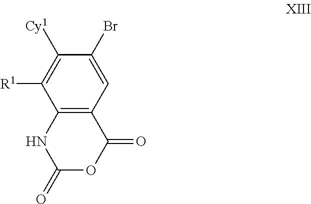
and the process comprises carbonylating a compound of Formula XV: 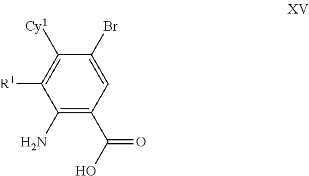
to form the compound of Formula XIII;
(11) the compound prepared is a compound of Formula XV: 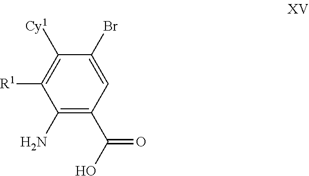
and the process comprises hydrolyzing a compound of Formula XVI: 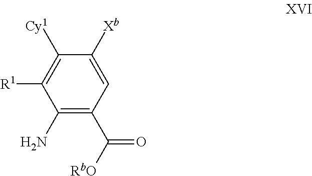
wherein
X^{b} is Cl, Br, or I;
R^{b} is C₁₋₃ alkyl;
to form the compound of Formula XV:
(12) the compound prepared is a compound of Formula XVI: 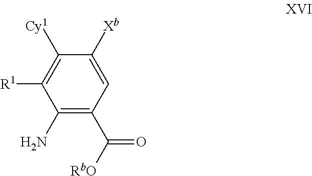
and the process comprises halogenating a compound of Formula XVII: 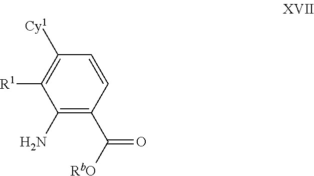
to form the compound of Formula XVI;
(13) the compound prepared is a compound of Formula XVII: 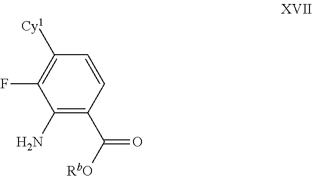
wherein
and the process comprises coupling a compound of Formula XVIII: 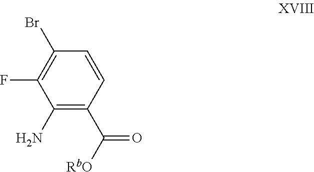
with a compound of Formula XIX: 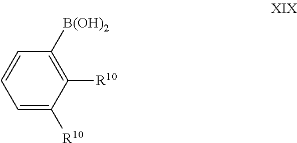
or an ester thereof:
wherein
each R¹⁰ is independently selected from C₁₋₃ alkyl and halo;
in the presence of a palladium catalyst to form the compound of Formula XVII; or
(14) the compound prepared is a compound of Formula XVIII: 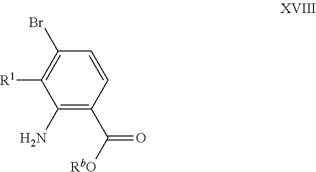
wherein
and the process comprises esterifying a compound of Formula XX: 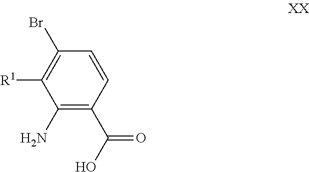
to form the compound of Formula XVIII.

8-34. (canceled)

35. The process of claim 7, wherein the process comprises separating atropisomers of the compound of Formula I, II, III, IV, V, VI, VII, VIII, IX, or X.

36-73. (canceled)

74. A process of preparing a compound of Formula I′: 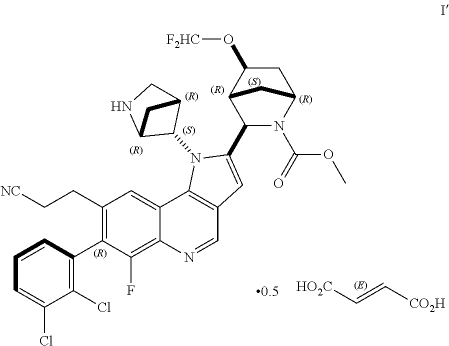
comprising reacting a compound of Formula I: 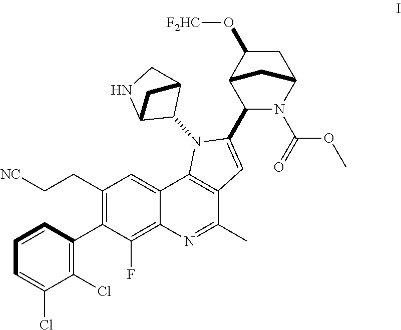
with fumaric acid to produce a compound of Formula I′.

75. The process of claim 1, wherein the compound of Formula I is methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt, hydrate, of solvate thereof.

76. The process of claim 1, wherein the compound of Formula I is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

77. The process of claim 1, wherein the compound of Formula I is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, or a pharmaceutically acceptable salt thereof.

78. (canceled)

79. The process of claim 7, wherein the compound of Formula II is tert-butyl 5-(8-(2-cyanoethyl)-7-(2,3-dichlorophenyl)-2-(5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate;
the compound of Formula III is tert-butyl 5-((6-(2-cyanoethyl)-7-(2,3-dichlorophenyl)-3-((5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate;
the compound of Formula IV is tert-butyl 5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate;
the compound of Formula V is methyl 5-(difluoromethoxy)-3-ethynyl-2-azabicyclo[2.2.1]heptane-2-carboxylate;
the compound of Formula VI is 4-((2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid;
the compound of Formula VII is tert-butyl 5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate;
the compound of Formula VIII is ethyl 4-chloro-6-(2-cyanoethyl)-7-(2,3-dichlorophenyl)-8-fluoro-2-methylquinoline-3-carboxylate;
the compound of Formula VIII is ethyl 6-(2-cyanoethyl)-7-(2,3-dichlorophenyl)-8-fluoro-4-hydroxy-2-methylquinoline-3-carboxylate;
the compound of Formula IX is tert-butyl 5-amino-2-azabicyclo[2.1.1]hexane-2-carboxylate;
the compound of Formula X is ethyl 4-chloro-6-(2-cyanovinyl)-7-(2,3-dichlorophenyl)-8-fluoro-2-methylquinoline-3-carboxylate;
the compound of Formula XI is ethyl 6-(2-cyanovinyl)-7-(2,3-dichlorophenyl)-8-fluoro-4-hydroxy-2-methylquinoline-3-carboxylate;
the compound of Formula XII is ethyl 6-bromo-7-(2,3-dichlorophenyl)-8-fluoro-4-hydroxy-2-methylquinoline-3-carboxylate;
the compound of Formula XIII is 6-bromo-7-(2,3-dichlorophenyl)-8-fluoro-2H-benzo[d][1,3]oxazine-2,4(1H)-dione;
the compound of Formula XIV is ethyl 3-oxobutanoate;
the compound of Formula XV is 3-amino-6-bromo-2′,3′-dichloro-2-fluoro-[1,1′-biphenyl]-4-carboxylic acid;
the compound of Formula XVI is methyl 3-amino-6-bromo-2′,3′-dichloro-2-fluoro-[1,1′-biphenyl]-4-carboxylate;
the compound of Formula XVII is methyl 3-amino-2′,3′-dichloro-2-fluoro-[1,1′-biphenyl]-4-carboxylate; and
the compound of Formula XVIII is methyl 2-amino-4-bromo-3-fluorobenzoate.

80. The process of claim 7, wherein the compound of Formula II is tert-butyl (1R,4R,5S)-5-(8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate;
the compound of Formula III is tert-butyl (1R,4R,5S)-5-((6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate;
the compound of Formula IV is tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate;
the compound of Formula V is methyl (1R,3R,4R,5S)-5-(difluoromethoxy)-3-ethynyl-2-azabicyclo[2.2.1]heptane-2-carboxylate;
the compound of Formula VI is 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid;
the compound of Formula VII is tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate; and
the compound of Formula IX is tert-butyl (1R,4R,5S)-5-amino-2-azabicyclo[2.1.1]hexane-2-carboxylate.

81. The process of claim 1, wherein the compound of Formula II is tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl;
the compound of Formula III is tert-butyl (1R,4R,5S)-5-(((Rₐ)-6-(2-cyanoethyl)-7-(2,3-dichlorophenyl)-3-(((1R,3R,4R,5S)-5-(difluoromethoxy)-2-(methoxycarbonyl)-2-azabicyclo[2.2.1]heptan-3-yl)ethynyl)-8-fluoro-2-methylquinolin-4-yl)amino)-2-azabicyclo[2.1.1]hexane-2-carboxylate;
the compound of Formula IV is tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate;
the compound of Formula VI is (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid;
the compound of Formula VII is tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate;
the compound of Formula VIII is ethyl (Rₐ)-4-chloro-6-(2-cyanoethyl)-7-(2,3-dichlorophenyl)-8-fluoro-2-methylquinoline-3-carboxylate; and
the compound of Formula VIII is ethyl (Rₐ)-6-(2-cyanoethyl)-7-(2,3-dichlorophenyl)-8-fluoro-4-hydroxy-2-methylquinoline-3-carboxylate.

82-117. (canceled)

118. A compound that is methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate.

119. The compound of claim 118, wherein the compound is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate.

120. (canceled)

121. A compound of Formula II or Formula III: 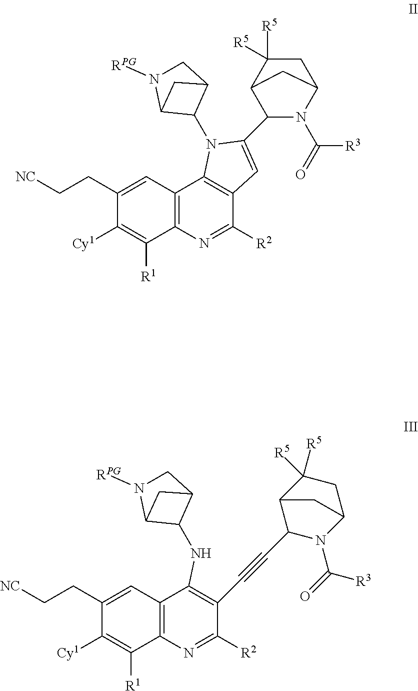
wherein
R^{PG} is a nitrogen protecting group;
Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
R¹ is halogen;
R² is C₁₋₃ alkyl optionally substituted with OH;
R³ is OR^{3A};
R^{3A} is selected from C₁₋₃ alkyl, C₂₋₃ alkenyl, and C₂₋₃ alkynyl;
each R⁵ is independently selected from H, D, halo, C₁₋₃ alkyl, OR^{a5}, C₁₋₃ haloalkyl, C₂₋₃ alkenyl, and C₂₋₃ alkynyl; and
each R^{a5} is independently selected from H, C₁₋₃ alkyl, and C₁₋₃ haloalkyl.

122-146. (canceled)

147. A compound that is a pharmaceutically acceptable salt of methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate, wherein the pharmaceutically acceptable salt is selected from fumarate, hemifumarate, hydrochloride, di-hydrochloride, L-tartrate, and phosphate.

148. The compound of claim 147 that is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate.

149. The compound of claim 148, which is characterized by an XRPD diffractogram having at least one of the following peaks expressed in degrees-2-theta at angles (±0.2 degrees) of 6.3, 6.7, 13.0, 17.1, 18.1, 19.4, 20.3, 23.8, 24.9, and 25.4.

150. (canceled)

151. The compound of claim 150, which is characterized by an XRPD diffractogram having peaks expressed in degrees-2-theta at angles (±0.2 degrees) of 6.3, 6.7, and 17.1.

152. (canceled)

153. The compound of claim 152, which is characterized by an XRPD diffractogram having peaks expressed in degrees-2-theta at angles (±0.2 degrees) of 6.3, 6.7, 13.0, 17.1, 23.8, and 24.9.

154-155. (canceled)

156. The compound of claim 148, which has a DSC thermogram characterized by an exotherm having a peak at about 238° C. and an exotherm having an onset at about 235° C.

157-159. (canceled)

160. A compound that is methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate hemimethanol.

161. The compound of claim 160, wherein the compound is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate hemimethanol.

162. The compound of claim 160, wherein the compound is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate hemifumarate hemimethanol.

163. A pharmaceutical composition comprising
a) the compound of claim 160;
b) a disintegrant;
c) a binder;
d) an anti-caking agent; and
e) a lubricant.

164. The pharmaceutical composition of claim 163, wherein the composition comprises two binders and two lubricants.

165-170. (canceled)

171. A pharmaceutical composition comprising:
a) the compound of claim 160;
b) sodium starch glycolate;
c) microcrystalline cellulose and lactose anhydrous;
d) colloidal silicon dioxide;
e) sodium stearyl fumarate; and
f) magnesium stearate.

172. A dosage form comprising the compound of claim 160.

173-174. (canceled)

175. A method of treating cancer in a subject in need thereof comprising administering to the subject a therapeutically effective amount of the compound of claim 160.

176. (canceled)

177. The method of claim 175, wherein the cancer is associated with expression or activity of a KRAS protein having a G12D mutation.

178. (canceled)

179. The method of claim 175, wherein the cancer is colorectal cancer, pancreatic cancer, or lung cancer.

180. (canceled)

181. The method of claim 175, wherein the cancer is pancreatic ductal cancer or non-small cell lung cancer (NCSLC).

182-183. (canceled)

184. The method of claim 175, wherein the cancer is metastatic.

185. (canceled)
